(19) United States
(12) Patent Application Publication
(10) Pub. No.: US 2025/0115651 A1
(43) Pub. Date: Apr. 10, 2025

(54) COMPOSITIONS AND METHODS FOR EPIGENETIC MODULATION OF NAV1.7

(71) Applicant: Navega Therapeutics, Inc., San Diego, CA (US)

(72) Inventors: Ana Maria MORENO COLLADO, San Diego, CA (US); Fernando ALEMAN GUILLEN, San Diego, CA (US)

(21) Appl. No.: 18/907,287

(22) Filed: Oct. 4, 2024

Related U.S. Application Data
(60) Provisional application No. 63/588,260, filed on Oct. 5, 2023.
(60) Provisional application No. 63/543,029, filed on Oct. 6, 2023.
(60) Provisional application No. 63/692,441, filed on Sep. 9, 2024.

(51) Int. Cl. C07K 14/47; A61K 38/00; A61K 48/00; A61P 25/02; C12N 15/86; C12N 15/88

(52) U.S. Cl. CPC C07K 14/4703; A61K 48/005; A61P 25/02; C12N 15/86; C12N 15/88; A61K 38/00; C07K 2319/10; C12N 2750/14143

(57) ABSTRACT

Described herein are epigenetic modulators that modulate expression of voltage gated sodium channels, such as Na_{V}1.7, without editing the genetic sequence. An epigenetic modulator may include a nucleic acid-binding agent that binds to a target sequence within a gene encoding the voltage gated sodium channel and an expression modulating agent that modulates expression of the gene. Also described herein are methods of treating inflammation, pain, or both using an epigenetic modulator of Na_{V}1.7.

### CROSS-REFERENCE

This application claims the benefit of U.S. Provisional Application No. 63/588,260, entitled “COMPOSITIONS AND METHODS FOR EPIGENETIC MODULATION OF NAV1.7,” filed Oct. 5, 2023, U.S. Provisional Application No. 63/543,029, entitled “COMPOSITIONS AND METHODS FOR EPIGENETIC MODULATION OF NAV1.7,” filed Oct. 6, 2023, and U.S. Provisional Application No. 63/692,441, entitled “COMPOSITIONS AND METHODS FOR EPIGENETIC MODULATION OF NAV1.7,” filed Sep. 9, 2024, each of which applications are herein incorporated by reference in their entireties for all purposes.

### GOVERNMENT SUPPORT

This invention was made with government support under U44NS122114 awarded by the National Institute of Health and government support under DISC2-13013 awarded by the California Institute for Regenerative Medicine. The government has certain rights in the invention.

### SEQUENCE LISTING

The instant application contains a Sequence Listing which has been submitted electronically in extensible Markup Language (XML) format and is hereby incorporated by reference in its entirety. Said XML copy, created on Oct. 4, 2024, is named “423157-711031 SL.xml” and is 189,732 bytes in size.

### BACKGROUND

Chronic pain affects between 19% to 50% of the world population, with more than 100 million people affected in the U.S. alone. Despite their side-effects and limited efficacy, opioids have been a preferred treatment for chronic pain among both private and VA prescribers in recent years. Opioids, however, are highly addictive, and over 130 Americans die each day due to an overdose. Thus, opioid overdose represents a threat that significantly impacts public health. Even though chronic pain is more prevalent than cancer, diabetes and cardiovascular disease combined, drug development for chronic pain has not undergone the remarkable progress seen in these other therapeutic areas. Despite decades of research, broad-acting, long-lasting, non-addictive and effective therapeutics for chronic pain remain elusive.

### SUMMARY

In various aspects, the present disclosure provides a zinc finger protein that includes (a) at least 90% sequence identity to SEQ ID NO: 2, (b) at least 98% sequence identity to SEQ ID NO: 3; and/or (c) at least 90% sequence identity to SEQ ID NO: 4.

In some aspects, the zinc finger protein includes a sequence having at least 95% sequence identity to SEQ ID NO: 2 or SEQ ID NO: 4. In some aspects, the zinc finger protein includes a sequence having at least 97% sequence identity to SEQ ID NO: 2 or SEQ ID NO: 4. In some aspects, the zinc finger protein includes a sequence of any one of SEQ ID NO: 1-SEQ ID NO: 4. In some aspects, the zinc finger protein has affinity to SCN9A. In some aspects the zinc finger protein has affinity for a polynucleotide sequence having at least 90% identity to any one of SEQ ID NO: 73-SEQ ID NO: 97.

In various aspects, the present disclosure provides an epigenetic modulator that includes the zinc finger protein linked to a repressor domain.

In some aspects, the epigenetic modulator includes a zinc finger protein with a sequence that has at least 90% sequence identity to any one of SEQ ID NO: 1-SEQ ID NO: 4 or SEQ ID NO: 99-SEQ ID NO: 169. In some aspects, the zinc finger protein includes a sequence having at least 95%, at least 97%, at least 98%, or 99% sequence identity to any one of SEQ ID NO: 1-SEQ ID NO: 4 or SEQ ID NO: 99-SEQ ID NO: 169. In some aspects, the zinc finger protein includes a sequence of any one of SEQ ID NO: 1-SEQ ID NO: 4 or SEQ ID NO: 99-SEQ ID NO: 169. In some aspects, the epigenetic modulator includes a zinc finger protein having affinity for a SCNA9. In some aspects, the epigenetic modulator includes a zinc finger protein having an affinity for a polynucleotide sequence having at least 90% sequence identity to any one of SEQ ID NO: 73-SEQ ID NO: 97. In some aspects, the epigenetic modulator includes a zinc finger protein having affinity for a polynucleotide sequence of any one of SEQ ID NO: 73-SEQ ID NO: 97. In some aspects, the epigenetic modulator includes a repressor domain that includes ZIM3, SID, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof. In some aspects, the repressor domain includes a sequence having at least 90% sequence identity to any one of SEQ ID NO: 5-SEQ ID NO: 20. In some aspects, the repressor domain includes any one of SEQ ID NO: 5-SEQ ID NO: 20. In some aspects, the repressor domain includes SEQ ID NO: 5 or SEQ ID NO: 9. In some aspects, the zinc finger protein in the epigenetic modulator is linked to the repressor domain via a peptide linker. In some aspects, the epigenetic modulator includes a second repressor domain. In some aspects, the second repressor domain includes ZIM3, SID, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof. In some aspects, the second repressor domain has a sequence that has at least 90% sequence identity to any one of SEQ ID NO: 5-SEQ ID NO: 20. In some aspects, the second repressor domain includes a sequence of any one of SEQ ID NO: 5-SEQ ID NO: 20. In some aspects, the second repressor domain includes a sequence of SEQ ID NO: 6 or SEQ ID NO: 7. In some aspects, the epigenetic modulator includes a sequence having at least 80% sequence identity to any one of SEQ ID NO: 25-SEQ ID NO: 36. In some aspects, the epigenetic modulator includes a sequence having at least 90% sequence identity to any one of SEQ ID NO: 25-SEQ ID NO: 36. In some aspects, the epigenetic modulator includes a sequence having at least 95% sequence identity to any one of SEQ ID NO: 25-SEQ ID NO: 36. In some aspects, the epigenetic modulator includes a sequence having of any one of SEQ ID NO: 25-SEQ ID NO: 36. In some aspects, the epigenetic modulator includes a sequence of SEQ ID NO: 25. In some aspects, the epigenetic modulator includes a sequence of SEQ ID NO: 26. In some aspects, the epigenetic modulator includes a sequence of SEQ ID NO: 27. In some aspects, the epigenetic modulator includes a sequence of SEQ ID NO: 28.

In various aspects, the present disclosure provides a polynucleotide encoding the zinc finger protein described herein. In some aspects, the polynucleotide includes a promoter.

In various aspects, the present disclosure provides a polynucleotide encoding the epigenetic modulator described herein. In some aspects, the polynucleotide includes a promoter.

In some aspects, the promoter includes a sequence having at least 90% sequence identity to any one of SEQ ID NO: 37-SEQ ID NO: 47 and SEQ ID NO: 98. In some aspects, the promoter includes a sequence of SEQ ID NO: 37-SEQ ID NO: 47 and/or SEQ ID NO: 98.

In various aspects, the present disclosure provides a delivery vector encapsulating the polynucleotides of the disclosure. In some aspects, the delivery vector includes a viral vector. In some aspects, the delivery vector includes a delivery-enhancing peptide. In some aspects, the delivery-enhancing peptide includes a protoxin, a jingzhaotoxina a theraphotoxin, a phlotoxin, Grammostola porter toxin, a huwentoxin, a Ceratogyrus cornuatus toxin, a heteropodatoxin, a heteroscodratoxin, and/or a penetration enhancing peptide. In some aspects, the delivery-enhancing peptide includes a sequence having at least 90% sequence identity to any one of SEQ ID NO: 48-SEQ ID NO: 72. In some aspects, the viral vector includes an AAV vector, a lentiviral vector, a Herpes Simplex Virus (HSV), or a rabies virus vector. In some aspects, the delivery vector includes a lipid nanoparticle that encapsulates DNA, mRNA or circular RNA coding for the epigenetic modulator.

In various aspects, the present disclosure provides a method for downregulating a Na_{V}1.7 in a cell. In some aspects, the methods involve expressing in the cell the epigenetic modulators described herein.

In various aspects, the present disclosure provides a method of treating a condition or a disease in a subject. In some aspects, the methods include expressing in the subject the epigenetic modulator described herein. In some aspects, the condition is associated with a Na_{V}1.7. In some aspects, the condition is pain, inflammation, and/or cancer. In some aspects, the condition is small-fiber neuropathy, back pain, rheumatoid arthritis, osteoarthritis, spinal stenosis, chronic cough, migraine, trigeminal neuralgia, erythromelalgia, and paroxysmal extreme pain disorder. In some aspects, the inflammation is associated with arthritis. In some aspects, the arthritis is rheumatoid arthritis or osteoarthritis. In some aspects, treating inflammation includes preventing inflammation. In some aspects, treating inflammation includes reducing inflammation. In some aspects, treating pain includes preventing pain. In some aspects, treating pain includes reducing pain. In some aspects, the pain is associated neuropathy, chemotherapy, or inflammation.

In various aspects, the present disclosure provides a method of delivering an epigenetic modulator as a purified protein to a cell. In some aspects, the method comprising administering the disclosed epigenetic modulator of to the cell. In some aspects, the epigenetic modulator is modified to enhance cellular uptake. In some aspects, the modification comprises fusion with a cell-penetrating peptide. In some aspects, the cell-penetrating peptide comprises TAT, polyarginine, or combinations thereof.

In various aspect, the present disclosure provides a composition for direct protein delivery, wherein the composition comprising the disclosed epigenetic modulator and a pharmaceutically acceptable carrier. In some aspects, the composition further comprises a cell-penetrating peptide fused to the epigenetic modulator. In some aspects, the composition further comprises a liposome encapsulating the epigenetic modulator.

In various aspects, the present disclosure provides a method of treating a condition in a subject, wherein the method comprising administering to the subject a therapeutically effective amount of the disclosed epigenetic modulator via direct protein delivery. In various aspects, the epigenetic modulator is administered by a route selected from the group consisting of intravenous injection, subcutaneous injection, intramuscular injection, aerosol administration and local administration to a target tissue, such as trigeminal ganglia or dorsal root ganglia. In various aspects, the condition is selected from the group consisting of pain, inflammation, cancer, chemotherapy-induced peripheral neuropathy, small-fiber neuropathy, back pain, rheumatoid arthritis, osteoarthritis, spinal stenosis, chronic cough, migraine, trigeminal neuralgia, erythromelalgia, and paroxysmal extreme pain disorder.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1C illustrates a repression of SCN9A in human cell lines to identify the lead candidate. (FIG. 1A) Eleven zinc finger proteins were transfected into HuH7 cells and the level of NaV1.7 repression was measured through qPCR using the ΔΔCt method. (FIG. 1B) The same ZF arrays were tested in IMR-90 and the repression levels were measured through qPCR using the ΔΔCt method. (FIG. 1C) Data obtained by the NCATS (branch of the NIH) with our lead ZF candidates. Repression levels obtained through ddPCR.

FIG. 2A-FIG. 2B illustrates an example for the isolation of a NaV1.7-specific promoter. (FIG. 2A) Seven NaV1.7 promoters driving the expression of mCherry were transfected into a high NaV1.7-expressing human cell line, HuH7. Two promoters with high transgene expression and short sequences being able to be packaged in an AAV were further studied in vivo. (FIG. 2B) Schematic of the reversal of pain in the CIPN model.

FIG. 3A-FIG. 3B illustrates results from the study of 5 promoters showing efficacy of different promoters. The data in each group represent individual biological replicates, with a sample size of 7 to 8 per group. Statistical analysis was performed using a two-way analysis of variance (ANOVA) followed by Dunnett's post hoc test. FIG. 3A illustrates statistical significance when compared to the mCherry control group. FIG. 3B illustrates statistical significance when compared to the naive group. If the p-value was less than 0.05 (p<0.05), it is considered statistically significant. Error bars represent the standard error of the mean (SEM).

FIG. 4A illustrates genotyping results of the 1197th base pair of the A) SCN9A mutant iPSC line and B) a healthy H1 embryonic stem cell (ESC) line with a wildtype SCN9A genomic sequences. SCN91 mutant iPSC line shows a consistent conflict between guanine and adenine base recall whereas the ESCs uniformly recall guanine without conflict.

FIG. 4B illustrates an experimental scheme to generate sensory neuronal-like cells positive for SCN9A expression. iPSCs were differentiated into premature sensory-like neurons by receiving daily media changes for seven days with each having a unique mixture of differentiation inducing factors.

FIG. 5A-FIG. 5F illustrates confocal images of iPSC-derived sensory neurons from IEM diagnosed patients. The sensory neurons were transduced with mCherry fluorescent protein. iPSCs underwent sensory neuronal differentiation for 19 days. (FIG. 5A) IEM iPSCs used to plate cells for sensory neuronal differentiation. (FIG. 5B) Day 2 of the differentiation process. (FIG. 5C) Day 6 of differentiation. Sensory neuronal-like cells transduced with 499_mCherry were imaged on day 19. (FIG. 5D) Brightfield channel. (FIG. 5E) mCherry channel. (FIG. 5F) Overlay of Brightfield and mCherry.

FIG. 6 illustrates a repression of Nav1.7 in IEM-iPSCs. Dots represent individual biological replicates; n=2.

FIG. 7A-FIG. 7B illustrates an ex vivo human DRG experiments. (FIG. 7A) A schematic method of the ex vivo human DRG experiments and (FIG. 7B) representative RNAscope images are shown.

FIG. 8 illustrates a RNAscope quantification of SCN9A in ex vivo human DRG. Dots represent individual biological replicates; n=10-33.

FIG. 9 illustrates a transgene expression in ex vivo cultures of mice DRG.

FIG. 10A illustrates a schematic of the measurement of AAV9 Nav1.7-1 transduction of whole DRG by confocal imaging.

FIG. 10B illustrates an expression of mCherry in mice following intrathecal injection of AAV9-Nav1.7-1-mCherry.

FIG. 10C illustrates a lack of transgene expression in liver using Nav1.7-specific promoter (499 promoter) via immunohistochemistry.

FIG. 10D illustrates a transgene expression in ex vivo human DRG cultures.

FIG. 11A-FIG. 11E illustrates an efficacy of ZF4-KRAB delivered through AAV9 in target engagement and pain amelioration in a carrageenan model of inflammatory pain. (FIG. 11A) Schematic of the overall strategy. (FIG. 11B) Schematic of the carrageenan-induced inflammatory pain model. (FIG. 11C) In vivo NaV1.7 repression levels determined by qPCR. (n=5; error bars are SEM; Student's t-test; ***p=0.0008). (FIG. 11D) The aggregate paw withdrawal latency was calculated as area under the curve (AUC) for both carrageenan and saline injected paws. Mice treated with ZF4-KRAB had significant increased paw-withdrawal latencies in carrageenan-injected paws (n=10; error bars are SEM; Student's t-test, ****p<0.0001). (FIG. 11E) The AUC of the aggregate PWL was calculated for both carrageenan- and saline-injected paws of mice at 3, 6, 12 and 44 weeks after intrathecal injection with AAV9-mCherry and AAV9-ZF4-KRAB. A significant increase in PWL is seen in the carrageenan-injected paws of mice injected with AAV9-ZF4-KRAB (dots represent individual biological replicates; n=5 to 8; error bars are SEM; Student's t test, **** P<0.0001).

FIG. 12 illustrates schematic of the paclitaxel-induced neuropathic pain model. Mice were IT injected with AAV9-mCherry, AAV9-ZF4-KRAB, or saline. Following baseline von Frey threshold testing at day 14, mice were then injected intraperitoneally (i.p.) with 8 mg/kg of paclitaxel at 14, 16, 18, 20 days after IT injection (32 mg/kg cumulative dosage). 21 (b,c) and 105 (d,e) days after IT injection, mice were tested for tactile allodynia via von Frey filaments and for cold allodynia via the application of acetone

FIG. 13A-FIG. 13D illustrates an In vivo efficacy of ZF4-KRAB in a chemotherapy-induced neuropathic pain model. (FIG. 13A) ZF4-KRAB reduces paclitaxel-induced tactile allodynia compared to the naïve group (n=8; error bars are SEM; Student's t-test; ***p=0.0007, ***p=0.0004). (FIG. 13B) ZF4-KRAB reduces paclitaxel-induced cold allodynia compared to the naïve group (n=8; error bars are SEM; Student's t-test; ****p<0.0001, **p=0.008). (FIG. 13C) ZF4-KRAB reduces paclitaxel-induced tactile allodynia 105 days after last paclitaxel injection compared to the naïve group (n=5-8; error bars are SEM; Student's t-test; ****p<0.0001, ***p=0.0001). (FIG. 13D) ZF4-KRAB reduces paclitaxel-induced cold allodynia compared to the naïve group (n=5-8; error bars are SEM; Student's t-test; ****p<0.0001). Similar proof-of-concept data were obtained using CRISPR-dCas9 (data not shown).

FIG. 14A-FIG. 14D illustrates an In vivo efficacy of ZF4-KRAB in a chemotherapy-induced neuropathic pain model. (FIG. 14A) ZF4-KRAB reduces paclitaxel-induced tactile allodynia compared to the mCherry group (n=8; error bars are SEM; Student's t-test; ***p=0.0007, ***p=0.0004). (FIG. 14B) ZF4-KRAB reduces paclitaxel-induced cold allodynia compared to the mCherry group (n=8; error bars are SEM; Student's t-test; ****p<0.0001, **p=0.008). (FIG. 14C) ZF4-KRAB reduces paclitaxel-induced tactile allodynia 105 days after last paclitaxel injection compared to the mCherry group (n=5-8; error bars are SEM; Student's t-test; ****p<0.0001, ***p=0.0001). (FIG. 14D) ZF4-KRAB reduces paclitaxel-induced cold allodynia compared to the mCherry group (n=5-8; error bars are SEM; Student's t-test; ****p<0.0001).

FIG. 15A-FIG. 15H illustrates safety assessments in mice. (FIG. 15A) Body weight of mice injected with AAV9-mCherry, AAV9-ZF4-KRAB are plotted (dots represent individual biological replicates; n=8 for mCherry and ZF4-KRAB groups; error bars are SEM; Student's t-test; n.s.=not significant). (FIG. 15B) Body temperature of mice injected with AAV9-mCherry, AAV9-ZF4-KRAB are plotted (error bars are SEM; Student's t-test). (FIG. 15C) Rotarod studies to determine motor coordination and balance of mice injected AAV9-ZF4-KRAB targeting NaV1.7 (error bars are SEM; Two-way ANOVA with Bonferroni post hoc test). (FIG. 15D) Grip strength of mice injected with AAV9-mCherry and AAV9-ZF4-KRAB are plotted (error bars are SEM; Two-way ANOVA with Bonferroni post hoc test). (FIG. 15E) Marble burying behavior of mice injected with AAV9-mCherry and AAV9-ZF4-KRAB are plotted. (FIG. 15F) Nest building behavior of mice injected with AAV9-mCherry and AAV9-ZF4-KRAB are plotted. (FIG. 15G) No significant olfactory detection changes were seen in mice injected with AAV9-ZF4-KRAB as compared to the control group (error bars are SEM; One-way ANOVA with Bonferroni post hoc test; n.s.=not significant). (FIG. 15H) A novel object recognition test showed comparable memory retention in mice injected with AAV9-ZF4-KRAB as compared to the control group (error bars are SEM; One-way ANOVA with Bonferroni post hoc test; n.s.=not significant).

### DETAILED DESCRIPTION

Described herein are compositions and methods to epigenetically modify gene expression without editing the genome. Also described herein are methods of treating pain, inflammation, or both using epigenetic modulation of gene expression. A composition for epigenetic modulation may comprise a nucleic acid-binding agent (e.g., a nucleic acid-binding protein), or a polynucleotide encoding the nucleic acid-binding agent, that binds to a target sequence within a genome. The target sequence may be a region (e.g., a coding region or a regulatory region) of a gene, such as a gene associated with pain or inflammation. In some embodiments, binding of the nucleic acid-binding agent to the target sequence may modulate (e.g., downregulate or upregulate) expression of the gene. In some embodiments, binding of the nucleic acid-binding agent to the target sequence may deliver an expression modulating agent (e.g., a transcriptional repressor, a transcriptional activator, or an epigenetic editor) to the gene, thereby modulating expression of the gene. The gene may encode a voltage-gated sodium channel (e.g., Na_{V}1.7) associated with pain. For example, the gene may be SCN9A. Nucleic acid-binding agents (e.g., SCN9A-binding agents) may include nucleic acid-binding proteins, such as zinc finger proteins. The nucleic acid-binding agent may be expressed with or linked to an expression modulating agent (e.g., ZIM3, SID, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof) that modulates expression of the gene.

Epigenetic modulation of gene expression using a composition of the present disclosure may (e.g., a composition modulating expression of Na_{V}1.7) may be used to treat pain, inflammation, or both in a subject. The pain or inflammation may be associated with a disorder (e.g., arthritis or a neurological disorder) or with a treatment of a disorder (e.g., chemotherapy for treatment of cancer). In some embodiments, a method of modulating gene expression may comprise delivering a polynucleotide encoding a nucleic acid-binding agent targeting a gene of interest and an expression modulating agent to a cell of the subject and expressing the nucleic acid-binding agent and the expression modulating agent in the cell, thereby modulating gene expression. Various methods may be used to deliver the polynucleotide to the cell of the subject. For example, the polynucleotide may be delivered using a viral vector (e.g., an adeno-associated virus (AAV) or a lentivirus vector) or a plasmid. In some embodiments, a method of modulating gene expression may comprise delivering the nucleic acid-binding agent and the expression modulating agent to a cell of the subject, thereby modulating gene expression. For example, the nucleic acid-binding agent and the expression modulating agent may be delivered on or in a nanoparticle, such as a lipid nanoparticle.

### Epigenetic Modulators

A composition of the present disclosure may comprise or encode an epigenetic modulator. An epigenetic modulator may modulate expression of a gene of interest without editing the genomic sequence. In some embodiments, an epigenetic modulator may comprise a nucleic acid-binding agent, an expression modulating agent, or both. The nucleic acid-binding agent may be linked to the expression modulating agent (e.g., expressed as a fusion protein), such that binding of the nucleic acid-binding agent to a target sequence delivers the expression modulating agent to the target sequence. Once in proximity to the target sequence, the expression modulating agent may modulate expression of a gene containing the target sequence.

### Zinc Finger Proteins

Zinc finger proteins (ZFP) comprise a DNA-binding domain made up of Cys2His2 zinc fingers. ZFPs constitute the largest individual family of transcriptional modulators encoded by the genomes of higher organisms.

In some embodiments, the nucleic acid compositions comprise a sequence encoding a ZFP. The ZFP may comprise a native or modified sequence. Non-limiting examples of ZFP sequences are provided in TABLE 1.

TABLE 1
Exemplary Zinc Finger Proteins

| SEQ | | Name |
|---|---|---|
| ID NO | Sequence | |
| SEQ ID | MAERPFQCRICMRNFSRSDVLSRHIRTHTGEKPFACDICGRKFAD | ZF9 |
| NO: 1 | SRDRKNHTKIHTGSQKPFQCRICMRNFSRSADLTRHIRTHTGEKP | (ZF191) |
| | FACDICGRKFADRSHLARHTKIHTGSQKPFQCRICMRNFSRSDNL | |
| | SEHIRTHTGEKPFACDICGRKFASKQYLIKHTKIHLRQKDAAR | |
| SEQ ID | LEPGEKPYKCPECGKSFSTSGNLVRHQRTHTGEKPYKCPECGKS | ZF8-PB |
| NO: 2 | FSTSGNLVRHQRTHMAERPFQCRICMRNFSRSDKLVRHIRTHTG | (8-ZF7-PB) |
| | EKPFACDICGRKFATSGHLSRHTKIHTGSQKPFQCRICMRNFSRS | |
| | DALSEHIRTHTGEKPFACDICGRKFAQNATRTKHTKIHTGSQKPF | |
| | QCRICMRNFSTSGHLSRHIRTHTGEKPFACDICGRKFAQSGDLTR | |
| | HTKIHLRQKDAAR | |
| SEQ ID | MAERPFQCRICMHNFSRSDVLSRHIRTHTGEKPFACDICGHKFA | ZF9 |
| NO: 3 | DSRDRKNHTKIHTGSQKPFQCRICMHNFSRSADLTRHIRTHTGE | (all H) |
| | KPFACDICGHKFADRSHLARHTKIHTGSQKPFQCRICMHNFSRSD | |
| | NLSEHIRTHTGEKPFACDICGHKFASKQYLIKHTKIHLRQKDAAR | |
| SEQ ID | LEPGEKPYKCPECGKSFSTSGNLVRHQRTHTGEKPYKCPECGKS | ZF8-PB |
| NO: 4 | FSTSGNLVRHQRTMAERPFQCRICMQNFSRSDKLVRHIRTHTGE | (all Q) |
| | KPFACDICGQKFATSGHLSRHTKIHTGSQKPFQCRICMQNFSRSD | |
| | ALSEHIRTHTGEKPFACDICGQKFAQNATRTKHTKIHTGSQKPFQ | |
| | CRICMQNFSTSGHLSRHIRTHTGEKPFACDICGQKFAQSGDLTRH | |
| | TKIHLRQKDAAR | |
| SEQ ID | MAERPFQCRICMRNFSTSGHLSRHIRTHTGEKPFACDICGRKFAD | ZF1 |
| NO: 99 | RSHLARHTKIHTGSQKPFQCRICMRNFSQSGDLTRHIRTHTGEKP | |
| | FACDICGRKFAYRWLRNNHTKIHTGSQKPFQCRICMRNFSRSDT | |
| | LSEHIRTHTGEKPFACDICGRKFARAQHLQQHTKIHLRQKDAAR | |
| SEQ ID | MAERPFQCRICMRNFSDRSHLSRHIRTHTGEKPFACDICGRKFAD | ZF2 |
| NO: | RSALARHTKIHTGSQKPFQCRICMRNFSQSSDLSRHIRTHTGEKP | |
| 100 | FACDICGRKFARSDDLTRHTKIHTGSQKPFQCRICMRNFSRSDDL | |
| | TRHIRTHTGEKPFACDICGRKFAQRSTLSSHTKIHLRQKDAAR | |
| SEQ ID | MAERPFQCRICMRNFSQSSDLSRHIRTHTGEKPFACDICGRKFAR | ZF3 |
| NO: | SDALARHTKIHTGSQKPFQCRICMRNFSQSGDLTRHIRTHTGEKP | |
| 101 | FACDICGRKFARSWGLQVHTKIHTGSQKPFQCRICMRNFSRSDH | |
| | LSQHIRTHTGEKPFACDICGRKFADSSTRKKHTKIHLRQKDAAR | |
| SEQ ID | MAERPFQCRICMRNFSDRSHLTRHIRTHTGEKPFACDICGRKFAQ | ZF4 |
| NO: | SGDLTRHTKIHTGSQKPFQCRICMRNFSRSDDLTRHIRTHTGEKP | |
| 102 | FACDICGRKFAQRSTLSSHTKIHTGSQKPFQCRICMRNFSRSDVL | |
| | SEHIRTHTGEKPFACDICGRKFARNQHRKTHTKIHLRQKDAAR | |
| SEQ ID | MAERPFQCRICMRNFSRSDNLSEHIRTHTGEKPFACDICGRKFAE | ZF5 |
| NO: | RANRNSHTKIHTGSQKPFQCRICMRNFSRSDNLARHIRTHTGEKP | |
| 103 | FACDICGRKFAWRGDRVKHTKIHTGSQKPFQCRICMRNFSDRSD | |
| | LSRHIRTHTGEKPFACDICGRKFARRTDLRRHTKIHLRQKDAAR | |
| SEQ ID | MAERPFQCRICMRNFSDRSDLSRHIRTHTGEKPFACDICGRKFAR | ZF6 |
| NO: | RTDLRRHTKIHTGSQKPFQCRICMRNFSDRSDLSRHIRTHTGEKP | |
| 104 | FACDICGRKFARSHHLKAHTKIHTGSQKPFQCRICMRNFSRSANL | |
| | ARHIRTHTGEKPFACDICGRKFARSDNLREHTKIHLRQKDAAR | |
| SEQ ID | MAERPFQCRICMRNFSRSAHLSRHIRTHTGEKPFACDICGRKFAT | ZF7 |
| NO: | SGHLSRHTKIHTGSQKPFQCRICMRNFSRSDALSEHIRTHTGEKP | |
| 105 | FACDICGRKFAQNATRTKHTKIHTGSQKPFQCRICMRNFSTSGHL | |
| | SRHIRTHTGEKPFACDICGRKFAQSGDLTRHTKIHLRQKDAAR | |
| SEQ ID | MAERPFQCRICMRNFSQSGDLTRHIRTHTGEKPFACDICGRKFA | ZF8 |
| NO: | QSGARNIHTKIHTGSQKPFQCRICMRNFSRSANLARHIRTHTGEK | |
| 106 | PFACDICGRKFARSDALARHTKIHTGSQKPFQCRICMRNFSDRSD | |
| | LSRHIRTHTGEKPFACDICGRKFARRTDLRRHTKIHLRQKDAAR | |
| SEQ ID | MAERPFQCRICMRNFSRSADLTRHIRTHTGEKPFACDICGRKFAQ | ZF10 |
| NO: | SGHLSRHTKIHTGSQKPFQCRICMRNFSQSSDLSRHIRTHTGEKPF | |
| 107 | ACDICGRKFAHRKSLSRHTKIHTGSQKPFQCRICMRNFSDRSDLS | |
| | RHIRTHTGEKPFACDICGRKFAQSSTRARHTKIHLRQKDAAR | |
| SEQ ID | MAERPFQCRICMRNFSDRSHLTRHIRTHTGEKPFACDICGRKFAR | ZF111 |
| NO: | SDDLTRHTKIHTGSQKPFQCRICMRNFSDRSHLTRHIRTHTGEKP | |
| 108 | FACDICGRKFARSDNLTRHTKIHTGSQKPFQCRICMRNFSQSGHL | |
| | ARHIRTHTGEKPFACDICGRKFAQKGTLGEHTKIHLRQKDAAR | |
| SEQ ID | MAERPFQCRICMRNFSRSAHLSRHIRTHTGEKPFACDICGRKFAQ | mZF1 |
| NO: | SGNLARHTKIHTGSQKPFQCRICMRNFSRSDAMSQHIRTHTGEK | |
| 109 | PFACDICGRKFARNASRTRHTKIHTGSQKPFQCRICMRNFSRSAN | |
| | LARHIRTHTGEKPFACDICGRKFADRSHLARHTKIHLRQKDAAR | |
| SEQ ID | MAERPFQCRICMRNFSRSANLARHIRTHTGEKPFACDICGRKFA | mZF2 |
| NO: | DSSDRKKHTKIHTGSQKPFQCRICMRNFSTSGSLSRHIRTHTGEK | |
| 110 | PFACDICGRKFAHSLSLKNHTKIHTGSQKPFQCRICMRNFSQSSD | |
| | LSRHIRTHTGEKPFACDICGRKFAWKWNLRAHTKIHLROKDAA | |
| | R | |
| SEQ ID | MAERPFQCRICMRNFSRSAHLSRHIRTHTGEKPFACDICGRKFAT | mZF3 |
| NO: | SGHLSRHTKIHTGSQKPFQCRICMRNFSRSDHLSQHIRTHTGEKP | |
| 111 | FACDICGRKFAASSTRTKHTKIHTGSQKPFQCRICMRNFSQSSHL | |
| | TRHIRTHTGEKPFACDICGRKFARSDNLTRHTKIHLRQKDAAR | |
| SEQ ID | MAERPFQCRICMRNFSDRSHLTRHIRTHTGEKPFACDICGRKFAD | mZF4 |
| NO: | RSHLARHTKIHTGSQKPFQCRICMRNFSRSDNLSEHIRTHTGEKP | |
| 112 | FACDICGRKFARSAALARHTKIHTGSQKPFQCRICMRNFSRSDTL | |
| | SQHIRTHTGEKPFACDICGRKFATRDHRIKHTKIHLRQKDAAR | |
| SEQ ID | MAERPFQCRICMRNFSRSDNLARHIRTHTGEKPFACDICGRKFAT | ZF119 |
| NO: | SGSLTRHTKIHTGSQKPFQCRICMRNFSRSDDLSKHIRTHTGEKP | |
| 113 | FACDICGRKFARSDALARHTKIHTGSQKPFQCRICMRNFSTSGHL | |
| | SRHIRTHTGEKPFACDICGRKFARSDALARHTKIHLRQKDAAR | |
| SEQ ID | MAERPFQCRICMRNFSRSDALSEHIRTHTGEKPFACDICGRKFAR | ZF102 |
| NO: | SADRTRHTKIHTGSQKPFQCRICMRNFSQSGHLARHIRTHTGEKP | |
| 114 | FACDICGRKFAHRSTLSRHTKIHTGSQKPFQCRICMRNFSDRSDL | |
| | SRHIRTHTGEKPFACDICGRKFADRSDLSRHTKIHLRQKDAAR | |
| SEQ ID | MAERPFQCRICMRNFSQSGDLTRHIRTHTGEKPFACDICGRKFA | ZF192 |
| NO: | QSGARNIHTKIHTGSQKPFQCRICMRNFSRSANLARHIRTHTGEK | |
| 115 | PFACDICGRKFARSDALARHTKIHTGSQKPFQCRICMRNFSDRSD | |
| | LSRHIRTHTGEKPFACDICGRKFARRTDLRRHTKIHLRQKDAAR | |
| | GS | |
| SEQ ID | MAERPFQCRICMRNFSRSDNLSRHIRTHTGEKPFACDICGRKFAH | ZF103 |
| NO: | SATRKRHTKIHTGSQKPFQCRICMRNFSRSDDLSKHIRTHTGEKP | |
| 116 | FACDICGRKFARSDALARHTKIHTGSQKPFQCRICMRNFSRSSHL | |
| | ARHIRTHTGEKPFACDICGRKFARSDALARHTKIHLRQKDAAR | |
| SEQ ID | MAERPFQCRICMRNFSRSDTLTRHIRTHTGEKPFACDICGRKFAR | ZF106 |
| NO: | SDARTNHTKIHTGSQKPFQCRICMRNFSRSSDLTRHIRTHTGEKP | |
| 117 | FACDICGRKFAQSGHLSRHTKIHTGSQKPFQCRICMRNFSRSDTL | |
| | TQHIRTHTGEKPFACDICGRKFADSSDLSRHTKIHLRQKDAAR | |
| SEQ ID | MAERPFQCRICMRNFSRSDDLTRHIRTHTGEKPFACDICGRKFAR | ZF108 |
| NO: | SDNLARHTKIHTGSQKPFQCRICMRNFSQSSALSRHIRTHTGEKP | |
| 118 | FACDICGRKFARSDHLSRHTKIHTGSQKPFQCRICMRNFSRSDSL | |
| | ARHIRTHTGEKPFACDICGRKFANRHHLTRHTKIHLRQKDAAR | |
| SEQ ID | MAERPFQCRICMRNFSDRSSLTTHIRTHTGEKPFACDICGRKFAD | ZF110 |
| NO: | RSHLARHTKIHTGSQKPFQCRICMRNFSRSDNLARHIRTHTGEKP | |
| 119 | FACDICGRKFAQSAHLARHTKIHTGSQKPFQCRICMRNFSQSGN | |
| | LARHIRTHTGEKPFACDICGRKFARSDHLSRHTKIHLRQKDAAR | |
| SEQ ID | MAERPFQCRICMRNFSRSDNLSEHIRTHTGEKPFACDICGRKFAQ | ZF112 |
| NO: | SANRNKHTKIHTGSQKPFQCRICMRNFSRSDNLARHIRTHTGEKP | |
| 120 | FACDICGRKFAWRGDRVKHTKIHTGSQKPFQCRICMRNFSDRSD | |
| | LSRHIRTHTGEKPFACDICGRKFARRTDLRRHTKIHLRQKDAAR | |
| SEQ ID | MAERPFQCRICMRNFSQSGSLSRHIRTHTGEKPFACDICGRKFAR | ZF113 |
| NO: | SDDLSRHTKIHTGSQKPFQCRICMRNFSRSDSLSQHIRTHTGEKPF | |
| 121 | ACDICGRKFARSGHLSRHTKIHTGSQKPFQCRICMRNFSDRSDLS | |
| | RHIRTHTGEKPFACDICGRKFAQSSTLARHTKIHLRQKDAAR | |
| SEQ ID | MAERPFQCRICMRNFSDQSTLRSHIRTHTGEKPFACDICGRKFAD | ZF114 |
| NO: | RSNLSRHTKIHTGSQKPFQCRICMRNFSRSDALSEHIRTHTGEKPF | |
| 122 | ACDICGRKFARSADRKRHTKIHTGSQKPFQCRICMRNFSQSGHL | |
| | ARHIRTHTGEKPFACDICGRKFATSSTLSKHTKIHLRQKDAAR | |
| SEQ ID | MAERPFQCRICMRNFSRSDHLSRHIRTHTGEKPFACDICGRKFAT | ZF115 |
| NO: | SSHLARHTKIHTGSQKPFQCRICMRNFSTSGNLARHIRTHTGEKP | |
| 123 | FACDICGRKFATSGNLVRHTKIHTGSQKPFQCRICMRNFSRSDHL | |
| | TRHIRTHTGEKPFACDICGRKFATSGHLVRHTKIHLROKDAAR | |
| SEQ ID | MAERPFQCRICMRNFSRSDNLARHIRTHTGEKPFACDICGRKFA | ZF117 |
| NO: | RSDHRKKHTKIHTGSQKPFQCRICMRNFSQSGNLARHIRTHTGE | |
| 124 | KPFACDICGRKFARSGNLRAHTKIHTGSQKPFQCRICMRNFSDSS | |
| | ALSTHIRTHTGEKPFACDICGRKFARSDHLSQHTKIHLRQKDAAR | |
| SEQ ID | MAERPFQCRICMRNFSERSDLSVHIRTHTGEKPFACDICGRKFAD | ZF118 |
| NO: | RDDLSRHTKIHTGSQKPFQCRICMRNFSRSDDRKVHIRTHTGEKP | |
| 125 | FACDICGRKFADRSHRIRHTKIHTGSQKPFQCRICMRNFSRSDNL | |
| | ARHIRTHTGEKPFACDICGRKFAQSGHLARHTKIHLRQKDAAR | |
| SEQ ID | MAERPFQCRICMRNFSQSSALTRHIRTHTGEKPFACDICGRKFAR | ZF801 |
| NO: | SDNLRAHTKIHTGSQKPFQCRICMRNFSQSGDLTRHIRTHTGEKP | |
| 126 | FACDICGRKFAQSGDLTRHTKIHTGSQKPFQCRICMRNFSQSGHL | |
| | VTHIRTHTGEKPFACDICGRKFATSGNLVRHTKIHLRQKDAAR | |
| SEQ ID | MAERPFQCRICMRNFSERSALATHIRTHTGEKPFACDICGRKFAD | ZF802 |
| NO: | RSALARHTKIHTGSQKPFQCRICMRNFSQSAHLSRHIRTHTGEKP | |
| 127 | FACDICGRKFARSDALARHTKIHTGSQKPFQCRICMRNFSQSGDL | |
| | TRHIRTHTGEKPFACDICGRKFATSGNLSRHTKIHLRQKDAAR | |
| SEQ ID | MAERPFQCRICMRNFSDRSALTRHIRTHTGEKPFACDICGRKFAQ | ZF803 |
| NO: | SGDLTRHTKIHTGSQKPFQCRICMRNFSRSDSLTRHIRTHTGEKP | |
| 128 | FACDICGRKFATSGNLTRHTKIHTGSQKPFQCRICMRNFSTSGHL | |
| | TRHIRTHTGEKPFACDICGRKFARSDALREHTKIHLRQKDAAR | |
| SEQ ID | MAERPFQCRICMRNFSQSGDLTRHIRTHTGEKPFACDICGRKFA | ZF804 |
| NO: | QSGDLTRHTKIHTGSQKPFQCRICMRNFSQSGHLSTHIRTHTGEK | |
| 129 | PFACDICGRKFATSGNLSRHTKIHTGSQKPFQCRICMRNFSQSGS | |
| | LRAHIRTHTGEKPFACDICGRKFARSDNLSKHTKIHLRQKDAAR | |
| SEQ ID | MAERPFQCRICMRNFSDRSHLTTHIRTHTGEKPFACDICGRKFAD | ZF805 |
| NO: | RSALTRHTKIHTGSQKPFQCRICMRNFSQSGDLTRHIRTHTGEKP | |
| 130 | FACDICGRKFARSDALAAHTKIHTGSQKPFQCRICMRNFSTSGNL | |
| | TRHIRTHTGEKPFACDICGRKFATSGHLTRHTKIHLRQKDAAR | |
| SEQ ID | MAERPFQCRICMRNFSRSDSLSVHIRTHTGEKPFACDICGRKFAQ | ZF806 |
| NO: | SGDLTRHTKIHTGSQKPFQCRICMRNFSTSGNLTRHIRTHTGEKP | |
| 131 | FACDICGRKFAQSTHLTSHTKIHTGSQKPFQCRICMRNFSQSGHL | |
| | ARHIRTHTGEKPFACDICGRKFAQLTHLNSHTKIHLRQKDAAR | |
| SEQ ID | MAERPFQCRICMRNFSDRSALSRHIRTHTGEKPFACDICGRKFAR | ZF807 |
| NO: | SQHLQTHTKIHTGSQKPFQCRICMRNFSDRSALSDHIRTHTGEKP | |
| 132 | FACDICGRKFAQSGNLARHTKIHTGSQKPFQCRICMRNFSRSDNL | |
| | ARHIRTHTGEKPFACDICGRKFAQSGHLARHTKIHLRQKDAAR | |
| SEQ ID | MAERPFQCRICMRNFSQSSNLSRHIRTHTGEKPFACDICGRKFAR | ZF808 |
| NO: | SDHLSTHTKIHTGSQKPFQCRICMRNFSRSDTLSEHIRTHTGEKPF | |
| 133 | ACDICGRKFAQSATLTRHTKIHTGSQKPFQCRICMRNFSRSDNLA | |
| | RHIRTHTGEKPFACDICGRKFAQSGALSRHTKIHLRQKDAAR | |
| SEQ ID | MAERPFQCRICMRNFSERGTLARHIRTHTGEKPFACDICGRKFAR | ZF809 |
| NO: | SDHRKTHTKIHTGSQKPFQCRICMRNFSDRSNLTRHIRTHTGEKP | |
| 134 | FACDICGRKFARSDNLARHTKIHTGSQKPFQCRICMRNFSRSDNL | |
| | ARHIRTHTGEKPFACDICGRKFATSANLVRHTKIHLRQKDAAR | |
| SEQ ID | MAERPFQCRICMRNFSDRSNLSRHIRTHTGEKPFACDICGRKFAR | ZF810 |
| NO: | SDNLTRHTKIHTGSQKPFQCRICMRNFSRSDTLSVHIRTHTGEKP | |
| 135 | FACDICGRKFADNSTRIKHTKIHTGSQKPFQCRICMRNFSRSDHL | |
| | SNHIRTHTGEKPFACDICGRKFADNRDRIKHTKIHLRQKDAAR | |
| SEQ ID | MAERPFQCRICMRNFSDRSNLSRHIRTHTGEKPFACDICGRKFAQ | ZF811 |
| NO: | LASRTKHTKIHTGSQKPFQCRICMRNFSRSDNLSAHIRTHTGEKP | |
| 136 | FACDICGRKFATSQNRITHTKIHTGSQKPFQCRICMRNFSRSDNL | |
| | SRHIRTHTGEKPFACDICGRKFAQSGNLVRHTKIHLRQKDAAR | |
| SEQ ID | MAERPFQCRICMRNFSQSGSLTRHIRTHTGEKPFACDICGRKFAR | ZF812 |
| NO: | SDALARHTKIHTGSQKPFQCRICMRNFSTSSNLTRHIRTHTGEKP | |
| 137 | FACDICGRKFATSGHLTRHTKIHTGSQKPFQCRICMRNFSRSDHL | |
| | SEHIRTHTGEKPFACDICGRKFADSRDRTKHTKIHLRQKDAAR | |
| SEQ ID | MAERPFQCRICMRNFSDRSALARHIRTHTGEKPFACDICGRKFA | ZF813 |
| NO: | QSGNLARHTKIHTGSQKPFQCRICMRNFSRSDNLARHIRTHTGE | |
| 138 | KPFACDICGRKFAQSGHLARHTKIHTGSQKPFQCRICMRNFSRSD | |
| | SLTQHIRTHTGEKPFACDICGRKFAQSSHLTRHTKIHLRQKDAAR | |
| SEQ ID | MAERPFQCRICMRNFSDRSHLSEHIRTHTGEKPFACDICGRKFAR | ZF814 |
| NO: | SDARKTHTKIHTGSQKPFQCRICMRNFSRSDTLSTHIRTHTGEKP | |
| 139 | FACDICGRKFADRSNLTRHTKIHTGSQKPFQCRICMRNFSRSDNL | |
| | ARHIRTHTGEKPFACDICGRKFARKDNRITHTKIHLRQKDAAR | |
| SEQ ID | MAERPFQCRICMRNFSQSGHLARHIRTHTGEKPFACDICGRKFA | ZF815 |
| NO: | QSGNLTRHTKIHTGSQKPFQCRICMRNFSQSGDLTRHIRTHTGEK | |
| 140 | PFACDICGRKFAQSGHLARHTKIHTGSQKPFQCRICMRNFSQSGH | |
| | LSQHIRTHTGEKPFACDICGRKFAQSTSLSKHTKIHLRQKDAAR | |
| SEQ ID | MAERPFQCRICMRNFSTSSNLSAHIRTHTGEKPFACDICGRKFAR | ZF816 |
| NO: | SDNRKKHTKIHTGSQKPFQCRICMRNFSQNSNLRRHIRTHTGEKP | |
| 141 | FACDICGRKFAQSGNLTRHTKIHTGSQKPFQCRICMRNFSQLGSL | |
| | SRHIRTHTGEKPFACDICGRKFALKSNLRRHTKIHLRQKDAAR | |
| SEQ ID | MAERPFQCRICMRNFSQSGNLQAHIRTHTGEKPFACDICGRKFA | ZF817 |
| NO: | QSGNLRAHTKIHTGSQKPFQCRICMRNFSQSGNLTRHIRTHTGEK | |
| 142 | PFACDICGRKFALKQHLRTHTKIHTGSQKPFQCRICMRNFSQSSA | |
| | LSRHIRTHTGEKPFACDICGRKFARSDSLRQHTKIHLRQKDAAR | |
| SEQ ID | MAERPFQCRICMRNFSRSDHLSEHIRTHTGEKPFACDICGRKFAR | ZF818 |
| NO: | SDHRKRHTKIHTGSQKPFQCRICMRNESTSSALSRHIRTHTGEKP | |
| 143 | FACDICGRKFARSDNLAAHTKIHTGSQKPFQCRICMRNFSTSGNL | |
| | ARHIRTHTGEKPFACDICGRKFAQSGDLTRHTKIHLRQKDAAR | |
| SEQ ID | MAERPFQCRICMRNFSRSWKLQAHIRTHTGEKPFACDICGRKFA | ZF819 |
| NO: | QSGNLTRHTKIHTGSQKPFQCRICMRNFSRSGHLSRHIRTHTGEK | |
| 144 | PFACDICGRKFARSDNLRAHTKIHTGSQKPFQCRICMRNFSTSSN | |
| | LSRHIRTHTGEKPFACDICGRKFARSDNLTQHTKIHLRQKDAAR | |
| SEQ ID | MAERPFQCRICMRNFSQSGDLTRHIRTHTGEKPFACDICGRKFAL | ZF820 |
| NO: | RTSLRKHTKIHTGSQKPFQCRICMRNFSTPSYLPTHIRTHTGEKPF | |
| 145 | ACDICGRKFADRSALARHTKIHTGSQKPFQCRICMRNFSQSSHLT | |
| | RHIRTHTGEKPFACDICGRKFARSDALARHTKIHLRQKDAAR | |
| SEQ ID | MAERPFQCRICMRNFSQSGNLSRHIRTHTGEKPFACDICGRKFAR | ZF821 |
| NO: | SDALRNHTKIHTGSQKPFQCRICMRNFSRSGNLARHIRTHTGEKP | |
| 146 | FACDICGRKFARSDNRITHTKIHTGSQKPFQCRICMRNFSRSDNL | |
| | SAHIRTHTGEKPFACDICGRKFARSDNRITHTKIHLRQKDAAR | |
| SEQ ID | MAERPFQCRICMRNFSRSDNLARHIRTHTGEKPFACDICGRKFA | ZF822 |
| NO: | QSANRTKHTKIHTGSQKPFQCRICMRNFSRSDNLARHIRTHTGE | |
| 147 | KPFACDICGRKFAQWNYRGSHTKIHTGSQKPFQCRICMRNFSQS | |
| | SDLSRHIRTHTGEKPFACDICGRKFAHRSNLSKHTKIHLRQKDAA | |
| | R | |
| SEQ ID | MAERPFQCRICMRNFSQSGHLARHIRTHTGEKPFACDICGRKFA | ZF823 |
| NO: | TSGNLARHTKIHTGSQKPFQCRICMRNFSQSGNLTRHIRTHTGEK | |
| 148 | PFACDICGRKFARSDNRITHTKIHTGSQKPFQCRICMRNFSRSAN | |
| | LSRHIRTHTGEKPFACDICGRKFAQSDTRITHTKIHLRQKDAAR | |
| SEQ ID | MAERPFQCRICMRNFSQRSNLSRHIRTHTGEKPFACDICGRKFAT | ZF824 |
| NO: | SGHLSRHTKIHTGSQKPFQCRICMRNFSRSDTLSEHIRTHTGEKPF | |
| 149 | ACDICGRKFAANSNRIKHTKIHTGSQKPFQCRICMRNFSRSDNLS | |
| | VHIRTHTGEKPFACDICGRKFARRDTRNNHTKIHLRQKDAAR | |
| SEQ ID | MAERPFQCRICMRNFSRSDTLSEHIRTHTGEKPFACDICGRKFAQ | ZF825 |
| NO: | NANRIKHTKIHTGSQKPFQCRICMRNFSRSDSLSQHIRTHTGEKP | |
| 150 | FACDICGRKFARSHNRKTHTKIHTGSQKPFQCRICMRNFSRSDNL | |
| | SRHIRTHTGEKPFACDICGRKFATNQNRITHTKIHLRQKDAAR | |
| SEQ ID | MAERPFQCRICMRNFSERGTLSRHIRTHTGEKPFACDICGRKFAR | ZF826 |
| NO: | SDHLSRHTKIHTGSQKPFQCRICMRNFSQSSTRKTHIRTHTGEKP | |
| 151 | FACDICGRKFATSSTLSNHTKIHTGSQKPFQCRICMRNFSRSDHL | |
| | SQHIRTHTGEKPFACDICGRKFAQSATRKTHTKIHLRQKDAAR | |
| SEQ ID | MAERPFQCRICMRNFSTSDHLSEHIRTHTGEKPFACDICGRKFAQ | ZF827 |
| NO: | SASRTKHTKIHTGSQKPFQCRICMRNFSQSGSLTRHIRTHTGEKP | |
| 152 | FACDICGRKFAQSGNLRKHTKIHTGSQKPFQCRICMRNFSDRSDL | |
| | SRHIRTHTGEKPFACDICGRKFAQSSDLSRHTKIHLRQKDAAR | |
| SEQ ID | MAERPFQCRICMRNFSRSDNLARHIRTHTGEKPFACDICGRKFA | ZF828 |
| NO: | RSDALARHTKIHTGSQKPFQCRICMRNFSRSDALSEHIRTHTGEK | |
| 153 | PFACDICGRKFARSSDRTKHTKIHTGSQKPFQCRICMRNFSRSDH | |
| | LSRHIRTHTGEKPFACDICGRKFARNQDRTNHTKIHLRQKDAAR | |
| SEQ ID | MAERPFQCRICMRNFSRSDTLSVHIRTHTGEKPFACDICGRKFAD | ZF829 |
| NO: | RSHLSRHTKIHTGSQKPFQCRICMRNFSRSDNLSVHIRTHTGEKP | |
| 154 | FACDICGRKFADRGNLTRHTKIHTGSQKPFQCRICMRNFSRSDNL | |
| | ARHIRTHTGEKPFACDICGRKFADSHHRITHTKIHLRQKDAAR | |
| SEQ ID | MAERPFQCRICMRNFSQSGNLSRHIRTHTGEKPFACDICGRKFAR | ZF830 |
| NO: | SDALATHTKIHTGSQKPFQCRICMRNFSRSDNLSTHIRTHTGEKP | |
| 155 | FACDICGRKFARSGNLSRHTKIHTGSQKPFQCRICMRNFSRSDNL | |
| | STHIRTHTGEKPFACDICGRKFATSSNRTKHTKIHLRQKDAAR | |
| SEQ ID | MAERPFQCRICMRNFSDRSALSRHIRTHTGEKPFACDICGRKFAR | ZF831 |
| NO: | SDALARHTKIHTGSQKPFQCRICMRNFSPCRYRLDHIRTHTGEKP | |
| 156 | FACDICGRKFARSANLTRHTKIHTGSQKPFQCRICMRNFSRSDNL | |
| | STHIRTHTGEKPFACDICGRKFADNSYLPRHTKIHLRQKDAAR | |
| SEQ ID | MAERPFQCRICMRNFSNSGDLTRHIRTHTGEKPFACDICGRKFAT | ZF832 |
| NO: | SGHLSRHTKIHTGSQKPFQCRICMRNFSRSDALSEHIRTHTGEKP | |
| 157 | FACDICGRKFAQNATRTKHTKIHTGSQKPFQCRICMRNFSQSGN | |
| | LTRHIRTHTGEKPFACDICGRKFAQSGDLGEHTKIHLRQKDAAR | |
| SEQ ID | MAERPFQCRICMRNFSRSDNLSRHIRTHTGEKPFACDICGRKFAR | ZF833 |
| NO: | SDARTRHTKIHTGSQKPFQCRICMRNFSDRSDLARHIRTHTGEKP | |
| 158 | FACDICGRKFARSSDLSRHTKIHTGSQKPFQCRICMRNFSRSDHL | |
| | SRHIRTHTGEKPFACDICGRKFADSQDRKTHTKIHLRQKDAAR | |
| SEQ ID | MAERPFQCRICMRNFSQSGSLTRHIRTHTGEKPFACDICGRKFAH | ZF834 |
| NO: | RQHLQTHTKIHTGSQKPFQCRICMRNFSDRSNLSRHIRTHTGEKP | |
| 159 | FACDICGRKFALKQVLVRHTKIHTGSQKPFQCRICMRNFSRSDSL | |
| | LRHIRTHTGEKPFACDICGRKFADRSNRRKHTKIHLRQKDAAR | |
| SEQ ID | MAERPFQCRICMRNFSRSDDLTAHIRTHTGEKPFACDICGRKFA | ZF835 |
| NO: | QSAHRKRHTKIHTGSQKPFQCRICMRNFSTSAHLARHIRTHTGE | |
| 160 | KPFACDICGRKFATSASRTRHTKIHTGSQKPFQCRICMRNFSTSG | |
| | HLTRHIRTHTGEKPFACDICGRKFARSDALAQHTKIHLRQKDAA | |
| | R | |
| SEQ ID | MAERPFQCRICMRNFSRSGNLTAHIRTHTGEKPFACDICGRKFA | ZF836 |
| NO: | QNANRIKHTKIHTGSQKPFQCRICMRNFSQSGSLTQHIRTHTGEK | |
| 161 | PFACDICGRKFATSQNLTRHTKIHTGSQKPFQCRICMRNFSTSDS | |
| | LLRHIRTHTGEKPFACDICGRKFATSHHLTRHTKIHLRQKDAAR | |
| SEQ ID | MAERPFQCRICMRNFSQSSTRKTHIRTHTGEKPFACDICGRKFAT | ZF837 |
| NO: | SSTRTKHTKIHTGSQKPFQCRICMRNFSDRSALSRHIRTHTGEKPF | |
| 162 | ACDICGRKFAQSGDLTRHTKIHTGSQKPFQCRICMRNFSRSDSLA | |
| | RHIRTHTGEKPFACDICGRKFATSGNLARHTKIHLRQKDAAR | |
| SEQ ID | MAERPFQCRICMRNFSTSGHLKEHIRTHTGEKPFACDICGRKFAQ | ZF838 |
| NO: | SGHLSRHTKIHTGSQKPFQCRICMRNFSQSSDLSRHIRTHTGEKPF | |
| 163 | ACDICGRKFAQSGNLSKHTKIHTGSQKPFQCRICMRNFSQSGHL | |
| | NRHIRTHTGEKPFACDICGRKFAQSGNLARHTKIHLRQKDAAR | |
| SEQ ID | MAERPFQCRICMRNFSTSSNRKTHIRTHTGEKPFACDICGRKFAQ | ZF839 |
| NO: | SANRITHTKIHTGSQKPFQCRICMRNFSQSGSLTRHIRTHTGEKPF | |
| 164 | ACDICGRKFALKQNRIKHTKIHTGSQKPFQCRICMRNFSTPSYLP | |
| | THIRTHTGEKPFACDICGRKFADRSALARHTKIHLRQKDAAR | |
| SEQ ID | MAERPFQCRICMRNFSRSGTLTRHIRTHTGEKPFACDICGRKFAQ | ZF840 |
| NO: | SGDLTRHTKIHTGSQKPFQCRICMRNFSQSGHLARHIRTHTGEKP | |
| 165 | FACDICGRKFARRSTRKSHTKIHTGSQKPFQCRICMRNFSQSGNL | |
| | ARHIRTHTGEKPFACDICGRKFAQSGNLARHTKIHLRQKDAAR | |
| SEQ ID | MAERPFQCRICMRNFSRSDHLSQHIRTHTGEKPFACDICGRKFAR | mZF1 |
| NO: | SAVRKNHTKIHTGSQKPFQCRICMRNFSRSDHLSEHIRTHTGEKP | |
| 166 | FACDICGRKFAQSHHRKTHTKIHTGSQKPFQCRICMRNFSDRSNL | |
| | SRHIRTHTGEKPFACDICGRKFALKQHLNEHTKIHLROKDAAR | |
| SEQ ID | MAERPFQCRICMRNFSDRSNLSRHIRTHTGEKPFACDICGRKFAR | mZF2 |
| NO: | SDDRKTHTKIHTGSQKPFQCRICMRNFSERGTLARHIRTHTGEKP | |
| 167 | FACDICGRKFAQSGHLSRHTKIHTGSQKPFQCRICMRNFSQSGHL | |
| | ARHIRTHTGEKPFACDICGRKFAVSHHLRDHTKIHLRQKDAAR | |
| SEQ ID | MAERPFQCRICMRNFSQSGNLARHIRTHTGEKPFACDICGRKFA | mZF4 |
| NO: | RLDILQQHTKIHTGSQKPFQCRICMRNFSRSDVLSEHIRTHTGEK | |
| 168 | PFACDICGRKFATRNGLKYHTKIHTGSQKPFQCRICMRNFSQSSD | |
| | LSRHIRTHTGEKPFACDICGRKFARKYYLAKHTKIHLRQKDAAR | |
| SEQ ID | LEPGEKPYKCPECGKSFSTSGNLVRHQRTHTGEKPYKCPECGKS | ZF8-P |
| NO: | FSTSGNLVRHQRTMAERPFQCRICMRNFSRSAHLSRHIRTHTGE | (8-ZF-P) |
| 169 | KPFACDICGRKFATSGHLSRHTKIHTGSQKPFQCRICMRNFSRSD | |
| | ALSEHIRTHTGEKPFACDICGRKFAQNATRTKHTKIHTGSQKPFQ | |
| | CRICMRNFSTSGHLSRHIRTHTGEKPFACDICGRKFAQSGDLTRH | |
| | TKIHLRQKDAAR | |

In some cases, a ZFP targeting Na_{V}1.7 comprises a sequence having at least 50%, at least 60%, at least 70%, at least 75%, at least 80%, at least 85%, at least 90%, at least 92%, at least 93%, at least 94%, at least 95%, at least 96%, at least 97%, at least 98%, at least 99%, or 100% sequence identity to a sequence of any of SEQ ID NO: 1-SEQ ID NO: 4. In some cases, the ZFP targeting Na_{V}1.7 comprises a sequence of any of SEQ ID NO: 1-SEQ ID NO: 4. In some cases, a ZFP targeting Na_{V}1.7 comprises a sequence having at least 90%, at least 92%, at least 93%, at least 94%, at least 95%, at least 96%, at least 97%, at least 98%, at least 99%, or 100% sequence identity to SEQ ID NO: 1. In some cases, a ZFP targeting Na_{V}1.7 comprises a sequence of SEQ ID NO: 1. In some cases, a ZFP targeting Na_{V}1.7 comprises a sequence having at least 90%, at least 92%, at least 93%, at least 94%, at least 95%, at least 96%, at least 97%, at least 98%, at least 99%, or 100% sequence identity to SEQ ID NO: 2. In some cases, a ZFP targeting Na_{V}1.7 comprises a sequence of SEQ ID NO: 2. In some cases, a ZFP targeting Na_{V}1.7 comprises a sequence having at least 90%, at least 92%, at least 93%, at least 94%, at least 95%, at least 96%, at least 97%, at least 98%, at least 99%, or 100% sequence identity to SEQ ID NO: 3. In some cases, a ZFP targeting Na_{V}1.7 comprises a sequence of SEQ ID NO: 3. In some cases, a ZFP targeting Na_{V}1.7 comprises a sequence having at least 90%, at least 92%, at least 93%, at least 94%, at least 95%, at least 96%, at least 97%, at least 98%, at least 99%, or 100% sequence identity to SEQ ID NO: 4. In some cases, a ZFP targeting Na_{V}1.7 comprises a sequence of SEQ ID NO: 4.

A zinc finger protein may bind to a target sequence. In some embodiments, the target sequence is a portion of a gene, such as a gene encoding Na_{V}1.7. Examples of zinc finger protein target sequences are provided in TABLE 2.

TABLE 2
Exemplary Target Sequences

| SEQ ID NO | Sequence | Gene Target |
|---|---|---|
| SEQ ID NO: 73 | AGTCTGCTTGCAGGCGGT | SCN9A |
| SEQ ID NO: 74 | CCAGCGGCGGCTGTCGGC | SCN9A |
| SEQ ID NO: 75 | GCCTGGGTGCCAGTGGCT | SCN9A |
| SEQ ID NO: 76 | TGGCTGCTAGCGGCAGGC | SCN9A |
| SEQ ID NO: 77 | AAGGAGAGGCCCGCGCCC | SCN9A |
| SEQ ID NO: 78 | GCAGGTGCACTGGGTGGG | SCN9A |
| SEQ ID NO: 79 | TGCCAGGGCGCGCCCGTG | SCN9A |
| SEQ ID NO: 80 | ACAGCCGCCGCTGGAGCG | SCN9A |
| SEQ ID NO: 81 | CCAGGAGAGGGCGCGGGC | SCN9A |
| SEQ ID NO: 82 | GGCGAGGTGATGGAAGGG | SCN9A |
| SEQ ID NO: 83 | GAGGGAGCTAGGGGTGGG | SCN9A |
| SEQ ID NO: 84 | AGTGCTAATGTTTCCGAG | SCN9A |
| SEQ ID NO: 85 | TAGACGGTGCAGGGCGGA | SCN9A |
| SEQ ID NO: 86 | GTGGGTGTGGCGGTTGAG | SCN9A |
| SEQ ID NO: 87 | GCCGCCGCTGGAGCGCTG | SCN9A |
| SEQ ID NO: 88 | GCGCCCGTGGAGGTAGCA | SCN9A |
| SEQ ID NO: 89 | GCCGCTGGAGCGCTGGCG | SCN9A |
| SEQ ID NO: 90 | GGTGTGGCGGTTGAGGCG | SCN9A |
| SEQ ID NO: 91 | GGGGAAGGAGAGGCCCGC | SCN9A |
| SEQ ID NO: 92 | GCGTCCCCTGAGCAACAG | SCN9A |
| SEQ ID NO: 93 | TCAGCCGAGCTGGCGGAA | SCN9A |
| SEQ ID NO: 94 | GCTGGAGCGCTGGCGACC | SCN9A |
| SEQ ID NO: 95 | GGTGGGGATGATCGGCGG | SCN9A |
| SEQ ID NO: 96 | TGGTTCCAGCAATGGGAG | SCN9A |
| SEQ ID NO: 97 | GGAGAGGGCGCGGGCCTC | SCN9A |

In some cases, a ZFP target sequence for modulating Na_{V}1.7 expression comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 73-SEQ ID NO: 97. In some cases, the ZFP target sequence for modulating Na_{V}1.7 expression comprises a sequence of any of SEQ ID NO: 73-SEQ ID NO: 97.

### Expression Modulating Agents

An expression modulating agent may modulate expression of a target molecule. In certain embodiments, the expression modulating agent comprises an activator that activates expression. In certain embodiments, the expression modulating agent comprises a repressor that represses expression. The expression modulating agent may comprise a transcription regulatory domain that has transcription repression activity (e.g., a repressor domain) or transcription activation activity (e.g., an activator domain). In some cases, the repressor domain comprises ZIM3. In some cases, the repressor domain comprises a Krueppel-associated box (KRAB) domain (recruitment of histone methyltransferases and deacetylases). Non-limiting examples of repressor domains are provided in TABLE 3.

TABLE 3
Exemplary Repressor Domains

| | SEQ ID | |
|---|---|---|
| Repressor | NO | Sequence |
| KRAB | SEQ ID | RTLVTFKDVFVDFTREEWKLLDTAQQIVYRNVMLENYKNL |
| (KOX1/ZNF10) | NO: 5 | VSLGYQLTKPDVILRLEKGEEPWLV |
| Sin3 | SEQ ID | MNIQMLLEAADYLERREREAEHGYASMLP |
| interacting | NO: 6 | |
| domain | | |
| (SID) | | |
| SID (x2) | SEQ ID | MNIQMLLEAADYLERREREAEHGYASMLPGGGSGGGSMNI |
| | NO: 7 | QMLLEAADYLERREREAEHGYASMLP |
| ZIM3 | SEQ ID | VTFEDVTVNFTQGEWQRLNPEQRNLYRDVMLENYSNLVSV |
| | NO: 8 | GQGETTKPDVILRLEQGKEPWL |
| KOX1 | SEQ ID | VTFKDVFVDFTREEWKLLDTAQQIVYRNVMLENYKNLVSL |
| | NO: 9 | GYQLTKPDVILRLEKGEEPWLV |
| ZNF554 | SEQ ID | VTFEDVSMDFSQEEWELLEPAQKNLYREVMLENYRNVVSL |
| | NO: 10 | EALKNQCTDVGIKEGPLSPAQT |
| ZNF264 | SEQ ID | VTFDDVAVTFTKEEWGQLDLAQRTLYQEVMLENCGLLVSL |
| | NO: 11 | GCPVPKAELICHLEHGQEPWT |
| ZNF324 | SEQ ID | MAFEDVAVYFSQEEWGLLDTAQRALYRRVMLDNFALVASL |
| | NO: 12 | GLSTSRPRVVIQLERGEEPWV |
| MeCP2 | SEQ ID | VQVKRVLEKSPGKLLVKMPFQASPGGKGEGGGATTSAQVM |
| | NO: 13 | VIKRPGRKRKAEADPQAIPKKRGRKPGSVVAAAAAEAKKK |
| | | AVKESSIRSVQETVLPIKKRKTRETVSIEVKEVVKPLLVSTLG |
| | | EKSGKGLKTCKSPGRKSKESSPKGRSSSASSPPKKEHHHHHH |
| | | HAESPKAPMPLLPPPPPPEPQSSEDPISPPEPQDLSSSICKEEK |
| | | MPRAGSLESDGCPKEPAKTQPMVAAAATTTTTTTTTVAEKY |
| | | KHRGEGERKDIVSSSMPRPNREEPVDSRTPVTERVS |
| MBD2b | SEQ ID | ARYLGNTVDLSSFDFRTGKMMPSKLQKNKQRLRNDPLNQN |
| | NO: 14 | KGKPDLNTTLPIRQTASIFKQPVTKVTNHPSNKVKSDPQRMN |
| | | EQPRQLFWEKRLQGLSASDVTEQIIKTMELPKGLQGVGPGS |
| | | NDETLLSAVASALHTSSAPITGQVSAAVEKNPAVWLNTSQP |
| | | LCKAFIVTDEDIRKQEERVQQVRKILEDALMADILSRAADTE |
| | | EMDIEMDSGDEAEF |
| SID | SEQ ID | MNIQMLLEAADYLERREREAEHGYASMLP |
| | NO: 15 | |
| HP1a | SEQ ID | MKEGENNKPREKSEGNKRKSSFSNSADDIKSKKKREQSNDI |
| | NO: 16 | ARGFERGLEPEKIIGATDSCGDLMFLMKWKDTDEADLVLAK |
| | | EANVKCPQIVIAFYEERLTWHAYPEDAENKEKESAKSEF |
| SIRT5 | SEQ ID | SSSMADFRKFFAKAKHIVIISGAGVSAESGVPTFRGAGGYWR |
| | NO: 17 | KWQAQDLATPLAFAHNPSRVWEFYHYRREVGSKEPNAGHR |
| | | AIAECETRLGKQGRRVWITQNIDELHRKAGTKNLLEIHGSLF |
| | | KTRCTSCGWAENYKSPICPALSGKGAPEPGTQDASIPVEKLP |
| | | RCEEAGCGGLLRPHWWFGENLDPAILEEVDRELAHCDLCL |
| | | WGTSSWYPAAFAPQVAARGVPVAEFNTETTPATNRFRFHFQ |
| | | GPCGTTLPEALACHENETVS |
| SETD8 | SEQ ID | SCDSTNAAIAKQALKKPIKGKQAPRKKAQGKTQQNRKLTDF |
| | NO: 18 | YPVRRSSRKSKAELQSEERKRIDELIESGKEEGKIDLIDGKGR |
| | | GVIATKQFSRGDFVVEYHGDLIEITDAKKREALYAQDPSTGC |
| | | YYYFQYLSKTYCVDATRETNRLGRLINHSKCGNCQTKLHDI |
| | | DGVPHLILIASRDIAAGEELLY DYGDRSKASIEAFPWLKHEF |
| HDT1 | SEQ ID | MEFWGIEVKSGKPVTVTPEEGILIHVSQASLGECKNKKGEFV |
| | NO: 19 | PLHVKVGNQNLVLGTLSTENIPQLFCDLVFDKEFELSHTWG |
| | | KGSVYFVGYKTPNIEPQGYSEEEEEEEEEVPAGNAAKAVAK |
| | | PKAKPAEVKPAVDDEEDESDSDGMDEDDSDGEDSEEEEPTP |
| | | KKPASSKKRANETTPKAPVSAKKAKVAVTPQKTDEKKKGG |
| | | KAANQSEF |
| SUPRD | SEQ ID | DLELRL |
| | NO: 20 | |

A composition of the present disclosure may comprise or encode a repressor domain of any of SEQ ID NO: 5-SEQ ID NO: 20, or a sequence having at least 50%, at least 60%, at least 70%, at least 75%, at least 80%, at least 85%, at least 90%, at least 92%, at least 95%, at least 97%, at least 98%, at least 99%, or 100% sequence identity to any one of SEQ ID NO: 5-SEQ ID NO: 20. The repressor domain may be a variant or combination of repressor domains of any of SEQ ID NO: 5-SEQ ID NO: 20. In some embodiments, the repressor domain comprises having at least 50%, at least 60%, at least 70%, at least 75%, at least 80%, at least 85%, at least 90%, at least 92%, at least 95%, at least 97%, at least 98%, at least 99%, or 100% sequence identity to ZIM3 (SEQ ID NO: 8). In some embodiments, the repressor domain comprises having at least 50%, at least 60%, at least 70%, at least 75%, at least 80%, at least 85%, at least 90%, at least 92%, at least 95%, at least 97%, at least 98%, at least 99%, or 100% sequence identity to KOX1 or a portion of KOX1 (e.g., SEQ ID NO: 5 or SEQ ID NO: 9).

In certain embodiments, the expression modulating agent comprises VP64 (recruitment of transcriptional activators), p65 (recruitment of transcriptional activators), p300 catalytic domain (histone acetyltransferase), TET1 catalytic domain (DNA demethylase), TDG (DNA demethylase), Ldb1 self-association domain (recruits enhancer-associated endogenous Ldb1), SAM activator (VP64, p65, HSF1) (recruits transcriptional activators), VPR (VP64, p65, Rta) (recruits transcriptional activators), Sin3a (recruitment of histone deacetylases), LSD1 (histone demethylase), SUV39H1 (histone methyltransferase), G9a (EHMT2) (histone methyltransferase), DNMT3a (DNA methyltransferase), or DNMT3a-DNMT3L (DNA methyltransferase), p16, p300, CD, SunTag, FOG1, DNMT3A, DNMT3L, DMT3, or a variant or combination thereof.

In certain embodiments, the expression modulating agent comprises KRAB (also referred to as KOX), SID, MBD2, MBD3, HPla, DNMT family (including DNMT1, DNMT3A, DNMT3B, DNMT3L, DNMT2A), Sin3a, Rb, MeCP2 (methyl-CpG binding protein 2), ROM2, AtHD2A, LSD1, SUV39H1, or G9a (EHMT2), or a variant or combination thereof. Variants of KRAB domains include ZIM3, ZNF554, ZNF264, ZNF324, ZNF354A, ZNF189, ZNF543, ZNP82, ZNF669, ZNF582, KOX1-MeCP2, ZNF30, ZNF680, ZNF331, ZNF33A, ZNF528, ZNF320, ZNF350, ZNF175, ZNF214, ZNF184, ZNF8, ZNF596, KOX1, ZNF37A, ZNF394, ZNF610, ZNF273, ZNF34, ZNF250, ZNF98, ZNF675, ZNF213, NLuc, ZFP28-2, ZNF224, or ZNF257, or a variant or combination thereof.

In certain embodiments, the expression modulating agent comprises a domain that recruits transcriptional activators, a histone acetyltransferase, a DNA demethylase, a domain that recruits enhancer-associated endogenous Ldb1, a domain that recruits histone methyltransferases and deacetylases, a domain that recruits histone deacetylases, a histone demethylase, a histone methyltransferase, a DNA methyltransferase, an acetylation domain, or a de-acetylation domain, or a combination thereof.

### Linkers

In certain embodiments, the transcription modulating agent is linked to the nucleic acid-binding agent. The transcription modulating agent may be positioned N- or C-terminal of the nucleic acid-binding agent. The domains may be linked via a peptide linker. The domains may be linked via a disulfide bond.

In some embodiments, the epigenetic modulator is linked to a nucleic acid binding domain via a peptide linker. Non-limiting examples of peptide linkers include (GGS)n (SEQ ID NO: 170), (GGGS)ₙ (SEQ ID NO: 171), (GGGGS)ₙ (SEQ ID NO: 172), (G)ₙ (SEQ ID NO: 173), (EAAAK)ₙ (SEQ ID NO: 174), A(EAAAK)ₙALEA(EAAAK)ₙA (SEQ ID NO: 175), PAPAP (SEQ ID NO: 176), AEAAAKEAAAKA (SEQ ID NO: 177), (Ala-Pro)ₓ (SEQ ID NO: 178), LE, GlySer-polyPro(Glyc)-polyPro(Glyc)-polyPro(Glyc), GlySer-polyPro-polyPro(Glyc)-polyPro, GlySer-polyPro-GlySer(Glyc)-polyPro, GlySer-polyPro-polyPro-polyPro, GlySer-polyPro-β2m-polyPro, GlySer-polyPro-β2m-GlySer, polyPro-β2m-GlySer-β2m-GlySer, GlySer-polyPro-β2m-GlySer-β2m-polyPro, GlySer-polyPro-Ub-GlySer, GlySer-polyPro-ZAG-polyPro, GlySer-GlySer-ZAG-GlySer-ZAG-polyPro, GlySer(Glyc)-GlySer(Glyc)-polyPro, (G₄S)₃-cTPR3-(G₄S)₃, (G₄S)₃-cTPR6-(G₄S)₃, (G₄S)₃-CTPR9-(G₄S)₃, (G₄S)₃-CTPR12-(G₄S)₃, and (G₄S)ₙ (SEQ ID NO: 172); wherein n is independently selected from 1 to 10, x is 10-34, polyPro is proline-rich hinge sequence from IgA1, polyPro(Glyc) is proline-rich hinge sequence from IgA1 with an embedded potential N-linked glycosylation site (Asn-Ser-Ser), β2m is β2-microglobulin, Ub is ubiquitin, ZAG is Zn-α2-glycoprotein, and cTPRX is consensus tetratricopeptide repeat sequence with X number of repeats. Additional examples of peptide linkers include amino acid sequences of MGS, GSS, GS, GGGSGT (SEQ ID NO: 179), GTGGGS (SEQ ID NO: 180), or GGGSGGGS (SEQ ID NO: 181).

### Epigenetic Modulators

A composition for epigenetic modulation of a target may comprise an epigenetic modulator. An epigenetic modulator of the present disclosure may comprise a zinc finger protein that binds a target sequence (e.g., a region of SCN9A) and a repressor domain (e.g., KRAB). In some embodiments, the zinc finger protein may comprise any one of SEQ ID NO: 1-SEQ ID NO: 4 or SEQ ID NO: 99-SEQ ID NO: 169. In some embodiments, the repressor domain may comprise a KRAB repressor domain (e.g., SEQ ID NO: 5 or SEQ ID NO: 9). The repressor domain may be positioned N-terminal of the zinc finger protein, or the repressor domain may be positioned C-terminal of the repressor domain. In some embodiments, the repressor domain and the zinc finger protein are separated by a linker (e.g., a linker comprising glycine and serine). In some embodiments, the epigenetic modulator may comprise a second repressor domain (e.g., a Sin3 interacting domain (SID) of SEQ ID NO: 6 or SEQ ID NO: 7). In some embodiments, the epigenetic modulator comprises a nuclear localization signal (e.g., SEQ ID NO: 21 (PKKKRKV) or SEQ ID NO: 22 (PKKKRKVLEPKKKRKVPGMAPKKKRKV)). The nuclear localization signal may localize the epigenetic modulator to the nucleus of a cell. In some embodiments, the epigenetic modulator comprises an affinity tag (e.g., a FLAG tag of SEQ ID NO: 23 (DYKDDDDK) or SEQ ID NO: 24 (MDYKDHDGDYKDHDIDYKDDDDK)).

Examples of epigenetic modulators comprising a zinc finger protein and a repressor domain are provided in TABLE 4.

TABLE 4
Exemplary Epigenetic Modulators

| SEQ | | | Gene |
|---|---|---|---|
| ID NO | Sequence | Name | Target |
| SEQ ID | MGSPKKKRKVLEPKKKRKVPGMAPKKKRKVG | NLS (3x) ZF9 | SCN9A |
| NO: 25 | SSMAERPFQCRICMRNFSRSDVLSRHIRTHTGEK | KRAB | |
| | PFACDICGRKFADSRDRKNHTKIHTGSQKPFQC | | |
| | RICMRNFSRSADLTRHIRTHTGEKPFACDICGRK | | |
| | FADRSHLARHTKIHTGSQKPFQCRICMRNFSRSD | | |
| | NLSEHIRTHTGEKPFACDICGRKFASKQYLIKHT | | |
| | KIHLRQKDAARGSRTLVTFKDVFVDFTREEWKL | | |
| | LDTAQQIVYRNVMLENYKNLVSLGYQLTKPDVI | | |
| | LRLEKGEEPWLV | | |
| SEQ ID | MGSPKKKRKVLEPKKKRKVPGMAPKKKRKVG | NLS (3x) | SCN9A |
| NO: 26 | SSLEPGEKPYKCPECGKSFSTSGNLVRHQRTHTG | 8ZF-PB | |
| | EKPYKCPECGKSFSTSGNLVRHQRTHMAERPFQ | KRAB | |
| | CRICMRNFSRSDKLVRHIRTHTGEKPFACDICGR | | |
| | KFATSGHLSRHTKIHTGSQKPFQCRICMRNFSRS | | |
| | DALSEHIRTHTGEKPFACDICGRKFAQNATRTK | | |
| | HTKIHTGSQKPFQCRICMRNFSTSGHLSRHIRTH | | |
| | TGEKPFACDICGRKFAQSGDLTRHTKIHLRQKD | | |
| | AARGSRTLVTFKDVFVDFTREEWKLLDTAQQIV | | |
| | YRNVMLENYKNLVSLGYQLTKPDVILRLEKGEE | | |
| | PWLV | | |
| SEQ ID | MGSPKKKRKVLEPKKKRKVPGMAPKKKRKVG | NLS (3x) ZF9 | SCN9A |
| NO: 27 | SSMAERPFQCRICMHNFSRSDVLSRHIRTHTGEK | (all H) KRAB | |
| | PFACDICGHKFADSRDRKNHTKIHTGSQKPFQC | | |
| | RICMHNFSRSADLTRHIRTHTGEKPFACDICGHK | | |
| | FADRSHLARHTKIHTGSQKPFQCRICMHNFSRSD | | |
| | NLSEHIRTHTGEKPFACDICGHKFASKQYLIKHT | | |
| | KIHLRQKDAARGSRTLVTFKDVFVDFTREEWKL | | |
| | LDTAQQIVYRNVMLENYKNLVSLGYQLTKPDVI | | |
| | LRLEKGEEPWLV | | |
| SEQ ID | MGSPKKKRKVLEPKKKRKVPGMAPKKKRKVG | NLS (3x) | SCN9A |
| NO: 28 | SSLEPGEKPYKCPECGKSFSTSGNLVRHQRTHTG | 8ZF-PB | |
| | EKPYKCPECGKSFSTSGNLVRHQRTMAERPFQC | (all Q) KRAB | |
| | RICMQNFSRSDKLVRHIRTHTGEKPFACDICGQK | | |
| | FATSGHLSRHTKIHTGSQKPFQCRICMQNFSRSD | | |
| | ALSEHIRTHTGEKPFACDICGQKFAQNATRTKH | | |
| | TKIHTGSQKPFQCRICMQNFSTSGHLSRHIRTHT | | |
| | GEKPFACDICGQKFAQSGDLTRHTKIHLRQKDA | | |
| | ARGSRTLVTFKDVFVDFTREEWKLLDTAQQIVY | | |
| | RNVMLENYKNLVSLGYQLTKPDVILRLEKGEEP | | |
| | WLV | | |
| SEQ ID | MDYKDHDGDYKDHDIDYKDDDDKMAPKKKR | FLAG (3x) | SCN9A |
| NO: 29 | KVGIHGVPAAMAERPFQCRICMRNFSRSDVLSR | NLS ZF9 | |
| | HIRTHTGEKPFACDICGRKFADSRDRKNHTKIHT | KRAB FLAG | |
| | GSQKPFQCRICMRNFSRSADLTRHIRTHTGEKPF | | |
| | ACDICGRKFADRSHLARHTKIHTGSQKPFQCRIC | | |
| | MRNFSRSDNLSEHIRTHTGEKPFACDICGRKFAS | | |
| | KQYLIKHTKIHLROKDAARGSRTLVTFKDVFVD | | |
| | FTREEWKLLDTAQQIVYRNVMLENYKNLVSLG | | |
| | YQLTKPDVILRLEKGEEPWLVDYKDDDDKRS | | |
| SEQ ID | MDYKDHDGDYKDHDIDYKDDDDKMAPKKKR | FLAG (3x) | SCN9A |
| NO: 30 | KVGIHGVPAALEPGEKPYKCPECGKSFSTSGNL | NLS 8ZF-PB | |
| | VRHQRTHTGEKPYKCPECGKSFSTSGNLVRHQR | KRAB FLAG | |
| | THMAERPFQCRICMRNFSRSDKLVRHIRTHTGE | | |
| | KPFACDICGRKFATSGHLSRHTKIHTGSQKPFQC | | |
| | RICMRNFSRSDALSEHIRTHTGEKPFACDICGRK | | |
| | FAQNATRTKHTKIHTGSQKPFQCRICMRNFSTSG | | |
| | HLSRHIRTHTGEKPFACDICGRKFAQSGDLTRHT | | |
| | KIHLRQKDAARGSRTLVTFKDVFVDFTREEWKL | | |
| | LDTAQQIVYRNVMLENYKNLVSLGYQLTKPDVI | | |
| | LRLEKGEEPWLVDYKDDDDKRS | | |
| SEQ ID | MDYKDHDGDYKDHDIDYKDDDDKMAPKKKR | FLAG (3x) | SCN9A |
| NO: 31 | KVGIHGVPAAMAERPFQCRICMHNFSRSDVLSR | NLS ZF9 | |
| | HIRTHTGEKPFACDICGHKFADSRDRKNHTKIHT | (all H) KRAB | |
| | GSQKPFQCRICMHNFSRSADLTRHIRTHTGEKPF | FLAG | |
| | ACDICGHKFADRSHLARHTKIHTGSQKPFQCRIC | | |
| | MHNFSRSDNLSEHIRTHTGEKPFACDICGHKFAS | | |
| | KQYLIKHTKIHLRQKDAARGSRTLVTFKDVFVD | | |
| | FTREEWKLLDTAQQIVYRNVMLENYKNLVSLG | | |
| | YQLTKPDVILRLEKGEEPWLVDYKDDDDKRS | | |
| SEQ ID | MDYKDHDGDYKDHDIDYKDDDDKMAPKKKR | FLAG (3x) | SCN9A |
| NO: 32 | KVGIHGVPAALEPGEKPYKCPECGKSFSTSGNL | NLS 8ZF-PB | |
| | VRHQRTHTGEKPYKCPECGKSFSTSGNLVRHQR | (all Q) KRAB | |
| | TMAERPFQCRICMQNFSRSDKLVRHIRTHTGEK | FLAG | |
| | PFACDICGQKFATSGHLSRHTKIHTGSQKPFQCR | | |
| | ICMQNFSRSDALSEHIRTHTGEKPFACDICGQKF | | |
| | AQNATRTKHTKIHTGSQKPFQCRICMQNFSTSG | | |
| | HLSRHIRTHTGEKPFACDICGQKFAQSGDLTRHT | | |
| | KIHLRQKDAARGSRTLVTFKDVFVDFTREEWKL | | |
| | LDTAQQIVYRNVMLENYKNLVSLGYQLTKPDVI | | |
| | LRLEKGEEPWLVDYKDDDDKRS | | |
| SEQ ID | MGSPKKKRKVLEPKKKRKVPGMAPKKKRKVG | NLS (3x) | SCN9A |
| NO: 33 | SSRTLVTFKDVFVDFTREEWKLLDTAQQIVYRN | KRAB ZF9 | |
| | VMLENYKNLVSLGYQLTKPDVILRLEKGEEPWL | SID (2x) | |
| | VGGGSGTMAERPFQCRICMRNFSRSDVLSRHIR | | |
| | THTGEKPFACDICGRKFADSRDRKNHTKIHTGS | | |
| | QKPFQCRICMRNFSRSADLTRHIRTHTGEKPFAC | | |
| | DICGRKFADRSHLARHTKIHTGSQKPFQCRICMR | | |
| | NFSRSDNLSEHIRTHTGEKPFACDICGRKFASKQ | | |
| | YLIKHTKIHLRQKDAARGTGGGSMNIQMLLEAA | | |
| | DYLERREREAEHGYASMLPGGGSGGGSMNIQM | | |
| | LLEAADYLERREREAEHGYASMLP | | |
| SEQ ID | MGSPKKKRKVLEPKKKRKVPGMAPKKKRKVG | NLS (3x) | SCN9A |
| NO: 34 | SSRTLVTFKDVFVDFTREEWKLLDTAQQIVYRN | KRAB 8ZF- | |
| | VMLENYKNLVSLGYQLTKPDVILRLEKGEEPWL | PB SID (2x) | |
| | VGGGSGTLEPGEKPYKCPECGKSFSTSGNLVRH | | |
| | QRTHTGEKPYKCPECGKSFSTSGNLVRHQRTHM | | |
| | AERPFQCRICMRNFSRSDKLVRHIRTHTGEKPFA | | |
| | CDICGRKFATSGHLSRHTKIHTGSQKPFQCRICM | | |
| | RNFSRSDALSEHIRTHTGEKPFACDICGRKFAQN | | |
| | ATRTKHTKIHTGSQKPFQCRICMRNFSTSGHLSR | | |
| | HIRTHTGEKPFACDICGRKFAQSGDLTRHTKIHL | | |
| | RQKDAARGTGGGSMNIQMLLEAADYLERRERE | | |
| | AEHGYASMLPGGGSGGGSMNIQMLLEAADYLE | | |
| | RREREAEHGYASMLP | | |
| SEQ ID | MGSPKKKRKVLEPKKKRKVPGMAPKKKRKVG | NLS (3x) | SCN9A |
| NO: 35 | SSRTLVTFKDVFVDFTREEWKLLDTAQQIVYRN | KRAB ZF9 | |
| | VMLENYKNLVSLGYQLTKPDVILRLEKGEEPWL | (all H) SID | |
| | VGGGSGTMAERPFQCRICMHNFSRSDVLSRHIR | (2x) | |
| | THTGEKPFACDICGHKFADSRDRKNHTKIHTGS | | |
| | QKPFQCRICMHNFSRSADLTRHIRTHTGEKPFAC | | |
| | DICGHKFADRSHLARHTKIHTGSQKPFQCRICM | | |
| | HNFSRSDNLSEHIRTHTGEKPFACDICGHKFASK | | |
| | QYLIKHTKIHLRQKDAARGTGGGSMNIQMLLEA | | |
| | ADYLERREREAEHGYASMLPGGGSGGGSMNIQ | | |
| | MLLEAADYLERREREAEHGYASMLP | | |
| SEQ ID | MGSPKKKRKVLEPKKKRKVPGMAPKKKRKVG | NLS (3x) | SCN9A |
| NO: 36 | SSRTLVTFKDVFVDFTREEWKLLDTAQQIVYRN | KRAB 8ZF- | |
| | VMLENYKNLVSLGYQLTKPDVILRLEKGEEPWL | PB (all Q) | |
| | VGGGSGTLEPGEKPYKCPECGKSFSTSGNLVRH | SID (2x) | |
| | QRTHTGEKPYKCPECGKSFSTSGNLVRHQRTMA | | |
| | ERPFQCRICMQNFSRSDKLVRHIRTHTGEKPFAC | | |
| | DICGQKFATSGHLSRHTKIHTGSQKPFQCRICMQ | | |
| | NFSRSDALSEHIRTHTGEKPFACDICGQKFAQNA | | |
| | TRTKHTKIHTGSQKPFQCRICMQNFSTSGHLSRH | | |
| | IRTHTGEKPFACDICGQKFAQSGDLTRHTKIHLR | | |
| | QKDAARGTGGGSMNIQMLLEAADYLERREREA | | |
| | EHGYASMLPGGGSGGGSMNIQMLLEAADYLER | | |
| | REREAEHGYASMLP | | |

In some embodiments, an epigenetic modulator may comprise a sequence having at least 50%, at least 60%, at least 70%, at least 75%, at least 80%, at least 85%, at least 90%, at least 92%, at least 95%, at least 97%, at least 98%, at least 99%, or 100% sequence identity to any one of SEQ ID NO: 25-SEQ ID NO: 36. In some embodiments, an epigenetic modulator comprises a sequence of any one of SEQ ID NO: 25-SEQ ID NO: 36.

### Polynucleotide Compositions

A composition of the present disclosure may comprise a polynucleotide encoding an epigenetic modulator or a component of an epigenetic modulator. For example, the polynucleotide may encode a nucleic acid-binding agent, an expression modulating agent (e.g., a repressor domain, an activator domain, or an epigenetic editor), or a combination thereof. In some embodiments, the nucleic acid-binding agent and the expression modulating agent may be expressed as a fusion protein.

### Coding Sequences

The polynucleotide may comprise one or more coding sequences. In some embodiments, a coding sequence may encode an epigenetic modulating agent of the present disclosure or a portion of an epigenetic modulating agent. For example, a coding sequence may encode a nucleic acid-binding agent (e.g., a zinc finger protein of any one of SEQ ID NO: 1-SEQ ID NO: 4 or SEQ ID NO: 99-SEQ ID NO: 169) or an expression modulating agent (e.g., a repressor domain of any one of SEQ ID NO: 5-SEQ ID NO: 20), or a combination thereof. In some embodiments, the polynucleotide encodes a polypeptide of any one of SEQ ID NO: 25-SEQ ID NO: 36. In some embodiments, the polynucleotide encodes a polypeptide comprising at least 50%, at least 60%, at least 70%, at least 75%, at least 80%, at least 85%, at least 90%, at least 92%, at least 95%, at least 97%, at least 98%, at least 99%, or 100% sequence identity to any one of SEQ ID NO: 25-SEQ ID NO: 36.

### Promoters

The polynucleotide may further comprise a promoter operably linked to a coding sequence encoding a component of an epigenetic modulator (e.g., a nucleic acid-binding agent, an expression modulating agent, or a combination thereof). The promoter may regulate transcription of the coding sequence. In some embodiments, the promoter may be a ubiquitous promoter that activates transcription of the coding sequence, independent of tissue type. In some embodiments, the promoter may be a cell-specific promoter (e.g., a neuronal-specific promoter) that activates transcription of the coding sequence in a cell type of interest. For example, the promoter may be specific for cells expressing Na_{V}1.7. Examples of promoters that may be included in a polynucleotide of the present disclosure are provided in TABLE 5.

TABLE 5
Exemplary Promoters

| | SEQ ID | |
|---|---|---|
| Name | NO | Sequence |
| | SEQ ID | CAGCCACTGCCCCAGACTCAGGGCTTTGCCATTGGTCCCCACCTCCTCTGCTC |
| | NO: 37 | CGGAGTTTTTCTCCAGCTCCCCACCAAGCCACACAAAGTGACTTCTCGGAAAC |
| | | ATTAGCCGATTCTGCTGAGCAGGAAGGGAGGAAAGGGATGATGGGGGCGGG |
| | | GGTGAGATAAGGGAAGGGCTCTTCTGGCTGCTGGACACACACACACACACAC |
| | | TCAAACACACACACGCCCCACCCAATGGGTGGCCGTGGATGGCAGGTCGTGC |
| | | AACCCCCTCCTCCGCCTTCTATTAGCGCATGGTGCAGAGGCTACAGCGTCGCC |
| | | ACCACCGCGCCCCTAGCTGGGTCCCCGCCCTGCGCCGCCCGCAGGAGTGGAG |
| | | AGAGGGAGGGAGGGAGGGAGCAAGGGGTGGGGACCCGGGCGCGCTGGGAG |
| | | GAGTGGAGGAGGCAAAGCGGCGCAGCTGCCCTCGGGGAGGCGGGGCTGCTA |
| | | CCTCCACGGGCGCGCCCTGGCAGGAGGGGCGCAGTCTGCTTGCAGGCGGTCG |
| | | CCAGCGCTCCAGCGGCGGCTGTCGGCTTTCCAATTCCGCCAGCTCGGCTGAG |
| | | GCTGGGCTAGCCTGGGTGCCAGTGGCTGC |
| | SEQ ID | CTTGAGCGTCACCGCCCCACTCGCGGTTGTGAGCAAAGCCCTACGGAAGAAA |
| | NO: 38 | CCAATTCCCAGCCTAGACTCTTCAGAGCCCAAGGTCGGGGAGGCGCTGGCCT |
| | | GGCGGTGTTGTCTGGCTCCCCAGCCACTGCCCCAGACTCAGGGCTTTGCCATT |
| | | GGTCCCCACCTCCTCTGCTCCGGAGTTTTTCTCCAGCTCCCCACCAAGCCACA |
| | | CAAAGTGACTTCTCGGAAACATTAGCCGATTCTGCTGAGCAGGAAGGGAGGA |
| | | AAGGGATGATGGGGGCGGGGGTGAGATAAGGGAAGGGCTCTTCTGGCTGCT |
| | | GGACACACACACACACACACTCAAACACACACACGCCCCACCCAATGGGTGG |
| | | CCGTGGATGGCAGGTCGTGCAACCCCCTCCTCCGCCTTCTATTAGCGCATGGT |
| | | GCAGAGGCTACAGCGTCGCCACCACCGCGCCCCTAGCTGGGTCCCCGCCCTG |
| | | CGCCGCCCGCAGGAGTGGAGAGAGGGAGGGAGGGAGGGAGCAAGGGGTGG |
| | | GGACCCGGGCGCGCTGGGAGGAGTGGAGGAGGCAAAGCGGCGCAGCTGCCC |
| | | TCGGGGAGGCGGGGCTGCTACCTCCACGGGCGCGCCCTGGCAGGAGGGGCG |
| | | CAGTCTGCTTGCAGGCGGTCGCCAGCGCTCCAGCGGCGGCTGTCGGCTTTCCA |
| | | ATTCCGCCAGCTCGGCTGAGGCTGGGCTAGCCTGGGTGCCAGTGGCTGC |
| | SEQ ID | GAGTGGAGAGAGGGAGGGAGGGAGGGAGCAAGGGGTGGGGACCCGGGCGC |
| | NO: 39 | GCTGGGAGGAGTGGAGGAGGCAAAGCGGCGCAGCTGCCCTCGGGGAGGCGG |
| | | GGCTGCTACCTCCACGGGCGCGCCCTGGCAGGAGGGGCGCAGTCTGCTTGCA |
| | | GGCGGTCGCCAGCGCTCCAGCGGCGGCTGTCGGCTTTCCAATTCCGCCAGCTC |
| | | GGCTGAGGCTGGGCTAGCCTGGGTGCCAGTGGCTGCTAGCGGCAGGCGTCCC |
| | | CTGAGCAACAGGAGCCCAGAGAAAAAGAAGCAGCCCTGAGAGAGCGCCGGG |
| | | GAAGGAGAGGCCCGCGCCCTCTCCTGGAGCCAGATTCTGCAGGTGCACTGGG |
| | | TGGGGATGATCGGCGGGCTAGGTTGCAAGTAAGTGCCTTTTCTTTTGCTGCTT |
| | | TCTTTATTTCTTTGTTTCCATCCAGGCCTCTTATGTGAGGAGCTGAAGAGGAA |
| | | TTAAAATATACAGGATGAAAAGGCGGCCGCCATGGTGAGCAAGGGCGAGGA |
| | | GGATAA |
| | SEQ ID | TCGAATTAACACAAGACAGAAGTCATATAAATCTTAAGAAGGTAGAAAATGT |
| | NO: 40 | TACTTCTAGAAAACTCCACCAAAAGTTGGTTGGATAATTTCAGGTCTTCTTTC |
| | | TGGAAAACGAAGAGTCACAAAGTCTATTTTTCTTTCTACTCCCAAGTTCCACT |
| | | CATTCTCCAATTAAATGTGACTCATAGGTGACTGAATGTCATGTTTTGGCCAC |
| | | TCTAAGCAAAGGGATTCCTTGGACTTGGGTACACATATTTTAAGAATATTGTA |
| | | TATATAAGAATATTGTATATATTCAACTGTCCATCTTGCTGCTTCTCTCTTACA |
| | | CCAACTTCATTTTGTAGCATCAGCTGCATAAGCTCTTGCTTTTCCCCCTATGCA |
| | | GACATCTATTCATCAGGCTCTGTGTACACTGGCAACATAGGAATAAACTGGA |
| | | TAAGGTTGCTGTTCCCAGAAGAATCTCAGAATATTGTATTTATTTAATGGCAT |
| | | CAGGAAAAACCAGTCCACCAATTTTAGAAAGAAAAAAGTCTTAAAGCTGCAC |
| | | ATTTTTATGAACTTAGACAATTACTATATTTTGACTATTATTGACTCTAATCTA |
| | | TTCCAAGAATTATACAGACAGAAAGAGCTTTGGTAACAGCCTACATATCAAT |
| | | ATTTACTGAACCAAGAACTATCACAAAACGTCTGTTGAAAATTTCCATGTTTT |
| | | AATGATGATGATGCTAATGGCAGCTATGATTTATTAAACACTTTCTATGTGCA |
| | | GGATGTAAGCTAAGTAATTTGTACACATTATCTTATTTAAACTTATTACAAGT |
| | | CTTATTTTCTTGAGGTAGATGTTATTATCCTATTTTCACAGACAGACTTATGCC |
| | | TTGAAGAATTTGGTAAAGTCACTAAGTAGTCAGTAGCCAAATCATGTATTCTT |
| | | AATCCTTATTCAATATTGTCCCCCTATAGAAGAAACCTTGAGTTTGTAACTTT |
| | | TCAGTAAGGCTATTTAGCTTGTGTCCTGAAGACACTCTCACCTATA |
| | SEQ ID | TCGAATTAACACAAGACAGAAGTCATATAAATCTTAAGAAGGTAGAAAATGT |
| | NO: 41 | TACTTCTAGAAAACTCCACCAAAAGTTGGTTGGATAATTTCAGGTCTTCTTTC |
| | | TGGAAAACGAAGAGTCACAAAGTCTATTTTTCTTTCTACTCCCAAGTTCCACT |
| | | CATTCTCCAATTAAATGTGACTCATAGGTGACTGAATGTCATGTTTTGGCCAC |
| | | TCTAAGCAAAGGGATTCCTTGGACTTGGGTACACATATTTTAAGAATATTGTA |
| | | TATATAAGAATATTGTATATATTCAACTGTCCATCTTGCTGCTTCTCTCTTACA |
| | | CCAACTTCATTTTGTAGCATCAGCTGCATAAGCTCTTGCTTTTCCCCCTATGCA |
| | | GACATCTATTCATCAGGCTCTGTGTACACTGGCAACATAGGAATAAACTGGA |
| | | TAAGGTTGCTGTTCCCAGAAGAATCTCAGAATATTGTATTTATTTAATGGCAT |
| | | CAGGAAAAACCAGTCCACCAATTTTAGAAAGAAAAAAGTCTTAAAGCTGCAC |
| | | ATTTTTATGAACTTAGACAATTACTATATTTTGACTATTATTGACTCTAATCTA |
| | | TTCCAAGAATTATACAGACAGAAAGAGCTTTGGTAACAGCCTACATATCAAT |
| | | ATTTACTGAACCAAGAACTATCACAAAACGTCTGTTGAAAATTTCCATGTTTT |
| | | AATGATGATGATGCTAATGGCAGCTATGATTTATTAAACACTTTCTATGTGCA |
| | | GGATGTAAGCTAAGTAATTTGTACACATTATCTTATTTAAACTTATTACAAGT |
| | | CTTATTTTCTTGAGGTAGATGTTATTATCCTATTTTCACAGACAGACTTATGCC |
| | | TTGAAGAATTTGGTAAAGTCACTAAGTAGTCAGTAGCCAAATCATGTATTCTT |
| | | AATCCTTATTCAATATTGTCCCCCTATAGAAGAAACCTTGAGTTTGTAACTTT |
| | | TCAGTAAGGCTATTTAGCTTGTGTCCTGAAGACACTCTCACCTATA |
| | SEQ ID | TCAATTATAACAGACATGTCTTAGTCTACCTTTTATTGCTGTAACTGAATACC |
| | NO: 42 | TGAGACTGGTTAAAGTATTCAATAAAGAAATTTATTTCTTAAAGTTCTGGAAG |
| | | CTGGGAAGTCTAAGGTGAAGGGTCCACATCTGGTGGGAACTTTCTTGCTGGT |
| | | GGGCACACCAGAGAGTCCCAAGGCAGCTCATGGCATCACATGGCGAGGGGT |
| | | GTTCACTGAGAGCCAAACTGGGTTTTATAATAAACCCACTCTTGTGATATCTA |
| | | ACTCACTCCCATGATAACCCATTAAGCCCTTACTCTATATTCCATTCATGAAG |
| | | GCAGAACCTTCATGACCTCATCACCTTTTAAAGGCTCTGCCTCTTAATACTGT |
| | | TACATTGGGAATTAAGTTCCAACATGAGTTTCAAAAGGGACAAACATTCAAA |
| | | CCACAGCAAAACATTAAAAAAAAAATTTTCTGATTAAAGAGGGAGCATACAT |
| | | ATTTTCAAGTGTTCTTAAGTGGAAAATAATGGATGTGAATGACTGGGCAAAT |
| | | TTAAGAGTGGCATGAGGAGTAATTTATTCTGCTAAATACGTAGCTTTAGGAAT |
| | | TTAAAAAAAAAAGCCATGTTACATTTAGTGAGACATCTGGAAATACTACATG |
| | | AGGATGAGCACTTTAAAACACTTCCCGCTTCATCAGTCAGTTTACCTGTCTCT |
| | | GATGGTAACAGCAATGAACTTTTTGTGAACAATGACTTTTTCTACGCCTGAAA |
| | | TAGATAAATATATAAAGTTGGCTCTCATCTGTTCCTATTCTTCCCTCGAAAAA |
| | | TTGAATCTCTCAGTCACTAACTTATTCGTAATGAAGAACTGCATTCCAGACAT |
| | | TTTTCTTAGACATCGTCTCCTGCTACACAGGATGTGAGGCAAATGAATATTTT |
| | | CCATGGAACTAATTCAGAGAGTAAACATATTTATAGATCCAGATCTAAGGTC |
| | | ATGGAGGAGCTCATTGCTTTGTAACTAAAATGTGTATTGATTGTATTGCTGTT |
| | | C |
| | SEQ ID | CAGTCTCAGTCACTGAAGTTTCCTTCCTTCAATAGCACATCTTACAAGTCAAG |
| | NO: 43 | AATGTAAGGTCCAAAGCCCCAGGACAAATCTCAGAAGCAAAGAAAGCAGCA |
| | | GGGAAAAGTAGACCCTGGGATTTGATTTCCTTCCGGTTATCTCTAAGCATCAT |
| | | TTCCATGATAGAAGGTGTGGAAGCAAATAATAAAAGTGCCCGTCACTAGTGT |
| | | TTATCCTGCAAAGTGGTCTGCCCTTTTGAGAGGCACCCTGCCCTATGGCCATC |
| | | CTTTGATTTCTTCCCTTGGTGGAAATTTCCTGTTTCTCTTTGAGAAAGATAATT |
| | | CAACCCTGTTTCCATTGTTCTTCCTCCCAGGCTGCTGTAGGAAAGCGCACGCA |
| | | CACACTCCACACAAAATGAATTTTTAAAAAATTTATTTTCACAGTCGCTCCTA |
| | | CCAGCTCTGAAATTCAGAACCCATATGACTGATGGCATATTCAGATAATCGG |
| | | GTCCCAGGTCTGGAAAAGCAGCCTTTTCCCCACGTTTCTTTCCCCACCTAGGA |
| | | CCTCCTCTGATTCTTCACTGCATCTTCGAAAGAAAATGTATTATTTGCTTGCCT |
| | | GGAAGACGCTGCAATTCAATTGATTTTATATATACATATATATAAAGAAAAC |
| | | AGAAAACATAGCCTAGATACCGGTCTTGAGCGTCACCGCCCCACTCGCGGTT |
| | | GTGAGCAAAGCCCTACGGAAGAAACCAATTCCCAGCCTAGACTCTTCAGAGC |
| | | CCAAGGTCGGGGAGGCGCTGGCCTGGCGGTGTTGTCTGGCTCCCCAGCCACT |
| | | GCCCCAGACTCAGGGCTTTGCCATTGGTCCCCACCTCCTCTGCTCCGGAGTTT |
| | | TTCTCCAGCTCCCCACCAAGCCACACAAAGTGACTTCTCGGAAACATTAGCC |
| | | GATTCTGCTGAGCAGGAAGGGAGGAAAGGGATGATGGGGGCGGGGGTGAGA |
| | | TAAGGGAAGGGCTCTTCTGGCTGCTGGACACACACACACACACACTCAAACA |
| | | CACACACGCCCCACCCAATGGGTGGCCGTGGATGGCAGGTCGTGCAACCCCC |
| | | TCCTCCGCCTTCTATTAGCGCATGGTGCAGAGGCTACAGCGTCGCCACCACCG |
| | | CGCCCCTAGCTGGGTCCCCGCCCTGCGCCGCCCGCAGGAGTGGAGAGAGGGA |
| | | GGGAGGGAGGGAGCAAGGGGTGGGGACCCGGGCGCGCTGGGAGGAGTGGA |
| | | GGAGGCAAAGCGGCGCAGCTGCCCTCGGGGAGGCGGGGCTGCTACCTCCAC |
| | | GGGCGCGCCCTGGCAGGAGGGGCGCAGTCTGCTTGCAGGCGGTCGCCAGCGC |
| | | TCCAGCGGCGGCTGTCGGCTTTCCAATTCCGCCAGCTCGGCTGAGGCTGGGCT |
| | | AGCCTGGGTGCCAGTGGCTGCTAGCGGCAGGCGTCCCCTGAGCAACAGGAGC |
| | | CCAGAGAAAAAGAAGCAGCCCTGAGAGA |
| hSyn | SEQ ID | CAGTGCAAGTGGGTTTTAGGACCAGGATGAGGCGGGGTGGGGGTGCCTACCT |
| | NO: 44 | GACGACCGACCCCGACCCACTGGACAAGCACCCAACCCCCATTCCCCAAATT |
| | | GCGCATCCCCTATCAGAGAGGGGGAGGGGAAACAGGATGCGGCGAGGCGCG |
| | | TGCGCACTGCCAGCTTCAGCACCGCGGACAGTGCCTTCGCCCCCGCCTGGCG |
| | | GCGCGCGCCACCGCCGCCTCAGCACTGAAGGCGCGCTGACGTCACTCGCCGG |
| | | TCCCCCGCAAACTCCCCTTCCCGGCCACCTTGGTCGCGTCCGCGCCGCCGCCG |
| | | GCCCAGCCGGACCGCACCACGCGAGGCGCGAGATAGGGGGGCACGGGCGCG |
| | | ACCATCTGCGCTGCGGCGCCGGCGACTCAGCGCTGCCTCAGTCTGCGGTGGG |
| | | CAGCGGAGGAGTCGTGTCGTGCCTGAGAGCGCAG |
| CMV | SEQ ID | CTCGAGGCGTTGACATTGATTATTGACTAGTTATTAATAGTAATCAATTACGG |
| | NO: 45 | GGTCATTAGTTCATAGCCCATATATGGAGTTCCGCGTTACATAACTTACGGTA |
| | | AATGGCCCGCCTGGCTGACCGCCCAACGACCCCCGCCCATTGACGTCAATAA |
| | | TGACGTATGTTCCCATAGTAACGCCAATAGGGACTTTCCATTGACGTCAATGG |
| | | GTGGAGTATTTACGGTAAACTGCCCACTTGGCAGTACATCAAGTGTATCATAT |
| | | GCCAAGTACGCCCCCTATTGACGTCAATGACGGTAAATGGCCCGCCTGGCAT |
| | | TATGCCCAGTACATGACCTTATGGGACTTTCCTACTTGGCAGTACATCTACGT |
| | | ATTAGTCATCGCTATTACCATGGTGATGCGGTTTTGGCAGTACATCAATGGGC |
| | | GTGGATAGCGGTTTGACTCACGGGGATTTCCAAGTCTCCACCCCATTGACGTC |
| | | AATGGGAGTTTGTTTTGGCACCAAAATCAACGGGACTTTCCAAAATGTCGTA |
| | | ACAACTCCGCCCCATTGACGCAAATGGGCGGTAGGCGTGTACGGTGGGAGGT |
| | | CTATATAAGCAGAGCTCTCTGGCTAACT |
| pJET | SEQ ID | GGGCGGAGTTAGGGCGGAGCCAATCAGCGTGCGCCGTTCCGAAAGTTGCCTT |
| | NO: 46 | TTATGGCTGGGCGGAGAATGGGCGGTGAACGCCGATGATTATATAAGGACGC |
| | | GCCGGGTGTGGCACAGCTAGTTCCGTCGCAGCCGGGATTTGGGTCGCGGTTC |
| | | TTGTTTGT |
| CAG | SEQ ID | GCGTTACATAACTTACGGTAAATGGCCCGCCTGGCTGACCGCCCAACGACCC |
| | NO: 47 | CCGCCCATTGACGTCAATAATGACGTATGTTCCCATAGTAACGCCAATAGGG |
| | | ACTTTCCATTGACGTCAATGGGTGGAGTATTTACGGTAAACTGCCCACTTGGC |
| | | AGTACATCAAGTGTATCATATGCCAAGTACGCCCCCTATTGACGTCAATGAC |
| | | GGTAAATGGCCCGCCTGGCATTATGCCCAGTACATGACCTTATGGGACTTTCC |
| | | TACTTGGCAGTACATCTACGTATTAGTCATCGCTATTACCATGGTCGAGGTGA |
| | | GCCCCACGTTCTGCTTCACTCTCCCCATCTCCCCCCCCTCCCCACCCCCAATTT |
| | | TGTATTTATTTATTTTTTAATTATTTTGTGCAGCGATGGGGGCGGGGGGGGGG |
| | | GGGGGGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGAAGCGCG |
| | | CGGCGGGCG |
| | SEQ ID | CAGCCACTGCCCCAGACTCAGGGCTTTGCCATTGGTCCCCACCTCCTCTGCTC |
| | NO: 98 | CGGAGTTTTTCTCCAGCTCCCCACCAAGCCACACAAAGTGACTTCTCGGAAAC |
| | | ATTAGCCGATTCTGCTGAGCAGGAAGGGAGGAAAGGGATGATGGGGGGGG |
| | | GGTGAGATAAGGGAAGGGCTCTTCTGGCTGCTGGACACACACACACACACAC |
| | | TCAAACACACACACGCCCCACCCAATGGGTGGCCGTGGATGGCAGGTCGTGC |
| | | AACCCCCTCCTCCGCCTTCTATTAGCGCATGGTGCAGAGGCTACAGCGTCGCC |
| | | ACCACCGCGCCCCTAGCTGGGTCCCCGCCCTGCGCCGCCCGCAGGAGTGGAG |
| | | AGAGGGAGGGAGGGAGGGAGCAAGGGGTGGGGACCCGGGCGCGCTGGGAG |
| | | GAGTGGAGGAGGCAAAGCGGCGCAGCTGCCCTCGGGGAGGCGGGGCTGCTA |
| | | CC |

In some embodiments, a polynucleotide may comprise a promoter having at least 50%, at least 60%, at least 70%, at least 75%, at least 80%, at least 85%, at least 90%, at least 92%, at least 95%, at least 97%, at least 98%, at least 99%, or 100% sequence identity to any one of SEQ ID NO: 37-SEQ ID NO: 47, or SEQ ID NO: 98. In some embodiments, the polynucleotide may comprise a promoter of any one of SEQ ID NO: 37-SEQ ID NO: 47, or SEQ ID NO: 98. The promoter may regulate transcription of one or more coding sequences of the polynucleotide.

In some embodiments, the promoter is specific to a target molecule so that the nucleic acid composition is specific for, or only expressed in, those cells expressing the target for increased therapeutic selectivity. As a non-limiting example, the target molecule is SCN9A.

In some embodiments, the promoter further increases the specificity of the AAV tropism for cells that express a target molecule. As a non-limiting example, the target molecule is SCN9A. Downregulating or upregulating a target molecule only in target molecule-expressing cells may reduce off-target effects and general toxicity. This is important to prevent the expression of the effectors (e.g., the epigenetic modulator) in immune cells such as glial cells, microglia, macrophages, astrocytes, etcetera, that can mediate an immune reaction against the gene therapy proposed herein.

In some embodiments, the promoter is specific to SCN9A (Na_{V}1.7) and is utilized to drive the repression of Na_{V}1.7 or other gene products (e.g., SEQ ID NO: 37-SEQ ID NO: 43). In some embodiments, the promoter is specific to a gene target described herein. In some embodiments, the promoter is a ubiquitous promoter (e.g., SEQ ID NO: 45-SEQ ID NO: 47, or SEQ ID NO: 98).

In some embodiments, some promoters can be induced with small molecules or other means. These inducible expression promoters include tetracycline responsive promoter, a glucocorticoid responsive promoter, an RU-486 responsive promoter, a peroxide inducible promoter and tamoxifen induced promoter.

Furthermore, there are promoters that can be induced when a pathology arises, such as injury or inflammation. Injury induced promoters include the galanin promoter specific to nociceptive afferent neurons. The inflammation-inducible promoter NF-κB could also be used for pain associated with inflammation.

Tandem promoters and combinations of promoters can also be used to prevent immune responses and create more cell-type specific expression.

In some embodiments, expression of the epigenetic modulator and/or transcription regulatory domain occurs upon a natural or physiological induction of the promoter.

In some embodiments, pan-neuronal gene promoters are used for modulation of gene expression in neurological diseases and for repression of pain-related genes. Non-limiting example promoters include the promoter of the microtubule-associated protein 2 (MAP-2), promoter of the Neuron specific enolase (NSE), promoter of the Choline Acetyltransferase (ChAT), promoter of the protein gene product 9.5 (PGP9.5) (also called ubiquitin-C-terminal hydrolase 1 (UCHL-1)), promoter of the human synapsin 1 (hSYN1) gene promoter, the promoter of the NeuN gene (Fox-3, Rbfox3, or Hexaribonucleotide Binding Protein-3), the promoter of the α-calcium/calmodulin-dependent protein kinase II [CaMKIIα]), the promoter of the Rheb gene (ras homolog enriched in brain), TRKA promoter (Tyrosine Kinase A). In some embodiments, the promoter is neuronal specific, such as pol II promoters, including Thy1 and H1xb9. Small latency-associated promoters from the herpesvirus pseudorabies virus can also be used for pan-neuronal expression of the effectors (ZFP) fused to a repressor domain.

Additional promoters include cytomegalovirus (CMV), SV40, elongation factor 1-alpha (EF1a) promoter, cytomegalovirus enhancer/chicken β-actin (CAG) promoter, jET promoter and herpes simplex virus (HSV).

In some embodiments, the promoter comprises a sequence at least about 90%, 91%, 92%, 93%, 94%, 95%, 96%, 97%, 98%, 99% or 100% identical to sequence of any of SEQ ID NO: 37-SEQ ID NO: 47, or SEQ ID NO: 98.

In some embodiments, the promoter is a SCN9A promoter.

In some embodiments, the promoter is naturally associated with a gene associated with a channelopathy, Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), pain (e.g., inflammatory pain, visceral pain, migraine pain, erythromelalgia pain, fibromyalgia pain, idiopathic pain, somatic pain), a neurological disease, dementia, Alzheimer's disease, Parkinson's disease, ALS, Multiple Sclerosis, a central nervous system ailment, or a combination thereof.

In some embodiments, the promoter is a promoter of a gene selected from SNCA, GBA, LRRK2, SOD1, ataxin-2, SCA2, BFD1, FUS, C9orf72, Brain-derived neurotrophic factor, Nerve growth factor, Neurotrophin, BCL11A, FMR1, DNM2, PrP, UBE3A, GYS1, and GFAP.

In some embodiments, the promoter is a pol II promoter (e.g., Thy1 and H1xb9), Small latency-associated promoter (e.g., from the herpesvirus pseudorabies virus), cytomegalovirus promoter, SV40, elongation factor 1-alpha (EF1a) promoter, cytomegalovirus enhancer/chicken β-actin (CAG) promoter, or herpes simplex virus (HSV) promoter.

### Other Components

A polynucleotide may further comprise an enhancer, an intron, a nuclear localization signal, an inverted terminal repeat (ITR), a terminator sequence, or combinations thereof. The polynucleotide may be included in a delivery vehicle, such as a vector.

Also provided herein are nucleic acid compositions comprising an adeno-associated virus modified with a sequence encoding a peptide specific for a protein product of a target molecule. Such compositions may be useful for targeting the nucleic acid to a cell expressing the target molecule for a targeted therapeutic approach. For example, the peptide specifically binds to the protein product of the target molecule. Non-limiting examples of specific binding include peptides that bind to the protein product of the target molecule with a high affinity, e.g., an affinity in the nanomolar range.

### Delivery Vectors

In some embodiments, a composition herein is delivered in a delivery vector. The delivery vector may be used to deliver an epigenetic modulator, a component of an epigenetic modulator, or a polynucleotide encoding an epigenetic modulator. In some embodiments, the delivery vector encapsulates a protein or a polynucleotide. In other embodiments, the composition is delivered complexed with cationic molecules.

In some embodiments, the composition is delivered to the subject via a vehicle. The vehicle may be a liposome, lipid nanoparticle, nanocapsule, or exosome.

In some embodiments, the composition is delivered via a viral vehicle. Non-limiting viral vehicles include, but are not limited to, retroviral vectors, lentiviral vectors, adenoviruses vectors, adeno-associated viral vectors (e.g., AAV1, AAV2, AAV3, AAV4, AAV5, AAV6, AAV7, AAV8, AAV9, AAV10, AAVhu68, AAVrh.10, AAVrh74, AAVDJ), and the like. In some cases, the vehicle is a recombinant adeno-associated virus (AAV). In some cases, the AAV is AAV9. AAV9 may be selected because it has the highest tropism for dorsal root ganglia (DRG) neurons where pain associated proteins such as Na_{V}1.7 are highly expressed. Further, AAV9 has been shown to be safe.

All delivery vehicles (viral vectors or non-viral) can have an improved tropism towards cells that express the protein product of a target molecule. For example, the vehicles can comprise a peptide that binds to the protein product of a target molecule. As a non-limiting example, the peptide binds to Na_{V}1.7 to target the nucleic acid to Na_{V}1.7 expressing cells.

### Targeting Moieties

In some embodiments, a composition herein is delivered via a viral vehicle, e.g., an AAV capsid described herein, with a nucleic acid sequence encoding a peptide targeting moiety.

In some embodiments, a composition herein is delivered via a non-viral delivery vehicle, such as, without limitation, a liposome, lipid nanoparticle, nanocapsule, or exosome. For some such embodiments, the vehicle may comprise and/or be connected to a targeting moiety, such as a small molecule or peptide targeting moiety.

In some embodiments, the targeting moiety comprises a peptide targeting moiety that binds to protein product of the target molecule in order to target the nucleic acid to a specific cell. In some cases, the target molecule is present on a target cell. In some cases, the target cell is associated with the disease or condition in the subject.

Non-limiting examples of peptide targeting moieties for use with viral and non-viral delivery methods include JNJ63955, m3-Huwentoxin-IV, Phlotoxin 1 (PhlTx1), Protoxin-II (ProTx-II), Ceratotoxin-1 (CcoTx1), Huwentoxin-IV (HwTx-IV), μ-TRTX-Pn3a, Jz-Tx-V, GsAFI, Tp1a (Protoxin-III), GpTx-1, HpTx1, Hm1a, and variants and combinations thereof. The peptide may originate from one or more of the following organisms: Thrixopelma pruriens, Pamphobeteus nigricolor, Chilobrachys jingzhao, Grammostola antracist, Phlogiellus genus, Thrixopelma pruriens, Grammostola anthracina, Selenocosmia huwena, Ceratogyrus cornuatus, Heteropoda venatoria, and/or Heteroscodra maculate.

TABLE 6 provides examples of peptides that may be used for targeting.

TABLE 6
Exemplary Peptide Targeting Moieties

| | SEQ ID | |
|---|---|---|
| Name | NO | Sequence |
| Protoxin-II | SEQ ID | YCQKWMWTCDSERKCCEGMVCRLWCKKKLW |
| | NO: 48 | |
| Protoxin-II variant | SEQ ID | GPYCQKWMQTCDSERKCCEGMVCRLWCKKKLL |
| (JNJ63955) | NO: 49 | |
| μ-TRTX-Pn3a | SEQ ID | DCRYMFGDCEKDEDCCKHLGCKRKMKYCAWDF |
| | NO: 50 | TFT |
| Jz-Tx-V | SEQ ID | YCQKWMWTCDSKRACCEGLRCKLWCRKII |
| | NO: 51 | |
| Jz-Tx-V variant | SEQ ID | YCQKWMWTCDSKRACCEGLRCKLWCRKEI |
| | NO: 52 | |
| GsAFI | SEQ ID | YCQKWLWTCDSERKCCEDMVCRLWCKKRL |
| | NO: 53 | |
| Phlotoxin 1 | SEQ ID | ACLGQWDSCDPKASKCCPNYACEWKYPWCRYKL |
| | NO: 54 | F |
| Phlotoxin 1 variant | SEQ ID | ACLGQWASCDPKASKCCPNYACEWKYPWCRYKL |
| | NO: 55 | F |
| Tpla (Protoxin-III) | SEQ ID | DCLKFGWKCNPRNDKCCSGLKCGSNHNWCKLHL |
| | NO: 56 | |
| GpTx-1 | SEQ ID | DCLGFMRKCIPDNDKCCRPNLVCSRTHKWCKYVF |
| | NO: 57 | |
| GpTx-1 variant | SEQ ID | DCLGFFRKCIPDNDKCCRPNLVCSRLHRWCKYVF |
| | NO: 58 | |
| GpTx-1 variant | SEQ ID | DCLGAMRKCIPDNDKCCRPNLVCSRTHKWCKYV |
| | NO: 59 | F |
| HwTx-IV | SEQ ID | ECLEIFKACNPSNDQCCKSSKLVCSRKTRWCKYQI |
| | NO: 60 | |
| m3-HwTx-IV variant | SEQ ID | GCLGIFKACNPSNDQCCKSSKLVCSRKTRWCKWQ |
| (m3-Huwentoxin-IV) | NO: 61 | I |
| CcoTx1 variant | SEQ ID | DCLGMFKSCDPENDKCCKRLVCSRSHRWCKWKL |
| | NO: 62 | |
| CcoTx1 variant | SEQ ID | ICLGMFKSCDPENDKCCKRLVCSRSHRWCKWKL |
| | NO: 63 | |
| HpTx1 | SEQ ID | DCGTIWHYCGTDQSECCEGWKCSRQLCKYVIDW |
| | NO: 64 | |
| Hm1a | SEQ ID | ECRYLFGGCSSTSDCCKHLSCRSDWKYCAWDGTF |
| | NO: 65 | S |
| AM-0422 | SEQ ID | YCQKW[Nle]WTCDSKRACC[Pra]GLRCKLWCRKEI |
| | NO: 66 | |
| OD-1 | SEQ ID | GVRDAYIADDKNCVYTCASNGYCNTECTKNGAES |
| | NO: 67 | GYCQWIGRYGNACWCIKLPDEVPIRIPGKCR |
| Penetration enhancing | SEQ ID | LLIILRRRIRKQAHAHSK |
| peptide | NO: 68 | |
| Penetration enhancing | SEQ ID | KETWWETWWTEWSQPKKKRKV |
| peptide | NO: 69 | |
| Penetration enhancing | SEQ ID | KLALKLALKALKAALKLA |
| peptide | NO: 70 | |
| Penetration enhancing | SEQ ID | LSTAADMQGVVTDGMASGLDKDYLKPDD |
| peptide | NO: 71 | |
| Penetration enhancing | SEQ ID | LLKKKCWLRCVMGECCKRESDCTQMWKQCYPG |
| peptide | NO: 72 | |

In some cases, the peptide comprises a sequence having at least 50%, at least 60%, at least 70%, at least 75%, at least 80%, at least 85%, at least 90%, at least 92%, at least 95%, at least 97%, at least 98%, at least 99%, or 100% sequence identity to a sequence of any of SEQ ID NO: 48-SEQ ID NO: 72. The peptides of TABLE 6 may bind to Nav1.7 and thus may be useful to provide AAV tropism to Nav1.7 expressing cells. In some cases, the peptide comprises ProTx-II, ProTx-III, the ProTx-II variant JNJ63955, HwTx-IV variant m3-HwTx-IV, CcoTx1 variant 2670, PhlTx1 variant D7A-PhlTx1, or JxTx-V variant AM-0422.

### AAV Vectors

Recombinant adeno-associated viruses (AAVs) are among the most commonly used vehicles for in vivo gene delivery. To enter a host cell, the virus 1) binds a receptor and glycan co-receptor on the cell surface, 2) is endocytosed, 3) progresses through the endosomal compartment, 4) escapes the endosome, and 5) traffics to the nucleus. Once inside the nucleus, the virus sheds its coat and its single-stranded genome is converted to a double-stranded one which the host cell can now use as a template for gene expression. The AAV receptor (AAVR, KIAA0319L) has been reported to be essential for AAV entry into cells, however, some recombinant AAVs can enter cells independent of AAVR. Glycosylation is also known to play a role in viral transduction efficiency. Changing the capsid composition of an AAV changes the ability of that AAV to enter cells. There are at least four major techniques currently used towards modifying and improving AAV tropism: rational engineering, directed evolution, evolutionary lineage analysis, and chemical conjugation.

One way to go about modifying a virus's tropism is to generate a large library of peptides to add onto the AAV surface and then characterize the resultant variants to determine their tropism. This method is labor intensive, requiring massive screening efforts as the library of peptides screened are more or less generated randomly so success is based on a numbers game. In a similar technique referred to as directed viral evolution, organs are collected from the first round of viral infection and screened to select viral variants with desired tropism (ex: brain-specific) for subsequent rounds of infection to further select even more specific tropism. However, some of these screening methods are not able to be translated between species.

Instead of using a random library screening, described herein is a rational design method to improve AAV tropism. Rational design strategies for AAV capsid engineering include peptide domain insertions and chemical biology approaches. One can add peptides which are known to specifically interact with cells of interest. By using binding peptides that bind to the protein encoding a target molecule, AAVs described herein have increased tropism towards target molecule expressing cells, generating more targeted strategies.

In some embodiments, the peptide to increase tropism binds to Na_{V}1.7. Peptides to increase Na_{V}1.7 tropism include: JNJ63955, m3-Huwentoxin-IV, Phlotoxin 1 (PhlTx1), Protoxin-II (ProTx-II), Ceratotoxin-1 (CcoTx1), Huwentoxin-IV (HwTx-IV), and those described elsewhere herein, e.g., a peptide of any of SEQ ID NO: 48-SEQ ID NO: 72 or a sequence at least about 90%, 91%, 92%, 93%, 94%, 95%, 96%, 97%, 98%, 99% or 100% identical to any of SEQ ID NO: 48-SEQ ID NO: 72.

### Target Molecules

In certain embodiments compositions described herein modulate expression of one or more target molecules associated with a disease or condition in a subject. In some cases, expression of the target molecule is activated. In some cases, expression of the target molecule is repressed.

The one or more target molecules may comprise DNA or RNA. In some embodiments, a target molecule comprises DNA. In some embodiments, a target molecule comprises a coding region of a gene. In some embodiments, a target molecule comprises DNA complementary to non-coding RNA. The non-coding RNA may be associated with a disease or condition described herein (e.g., pain). For example, the non-coding RNA is associated with neuropathic pain such as spinal nerve ligation, spared nerve injury, chronic constriction injury, or diabetic neuropathy, or a combination thereof.

In some embodiments, the non-coding RNA comprises a SCN9A natural antisense transcript (NAT), a Kcna2 antisense RNA, H19, Gm21781, MRAK009713, uc.48+, NONRATT021972, BC168687, Speer7-ps, Uc007pbc.1, XLOC_041439, Mlxipl, Rn50_X_0739.1, CCAT1, rno circ 0004058, rno_circRNA_007512, or Egr2 antisense RNA, or a combination thereof.

In some embodiments, the one or more target molecules comprises a nucleic acid associated with a disease or condition described here. In some embodiments, the one or more target molecules comprises a nucleic acid associated with pain. Pain includes neuropathic pain, inflammatory pain, visceral pain, migraine pain, erythromelalgia pain, fibromyalgia pain, idiopathic pain, and somatic pain. For example, the target molecule may be a SCN9A gene encoding Na_{V}1.7. In some embodiments, the target molecule comprises a sequence of any one of SEQ ID NO: 73-SEQ ID NO: 97.

In some embodiments, the one or more target molecules comprises a nucleic acid associated with a channelopathy. In some cases, the one or more target molecules comprises a nucleic acid encoding a channel. The channel may be an ion channel, e.g., sodium channel, potassium channel, calcium channel, and/or chloride channel.

In some embodiments, the one or more target molecules comprises a nucleic acid associated with a neurological disease. In some embodiments, the one or more target molecules comprises a nucleic acid associated with dementia. In some embodiments, the one or more target molecules comprises a nucleic acid associated with Alzheimer's disease. In some embodiments, the one or more target molecules comprises a nucleic acid associated with Parkinson's disease. In some embodiments, the one or more target molecules comprises a nucleic acid associated with Huntington's disease. In some embodiments, the one or more target molecules comprises a nucleic acid associated with schizophrenia. In some embodiments, the one or more target molecules comprises a nucleic acid associated with Amyotrophic lateral sclerosis (ALS). In some embodiments, the one or more target molecules comprises a nucleic acid associated with Multiple Sclerosis. In some embodiments, the one or more target molecules comprises a nucleic acid associated with a central nervous system ailment. In some embodiments, the one or more target molecules comprises a nucleic acid associated with Dravet syndrome, an Epilepsy syndrome, Familial hemiplegic migraine, Ohtahara syndrome, West syndrome, Lennox-Gastaut syndrome, sodium channel myotonia, autism, Long QT syndrome, Brugada syndrome, or Progressive cardiac conduction disease (also called Lenègre disease), or a combination thereof.

### Genes Involved in Pain

The human genome encodes genes that can confer protection to unnecessary pain. Genetic studies have correlated a hereditary loss-of-function mutation in a human-voltage gated sodium channel—Na_{V}1.7 (SCN9A)—with a rare genetic disorder, which leads to insensitivity to pain without other neurodevelopmental alterations. Thus, this sodium channel has been an attractive target for developing chronic pain therapies. However, efforts to develop selective small molecule inhibitors have been hampered due to the high sequence identity between Na_{V} subtypes, and in fact, many small-molecule drugs targeting Na_{V}1.7 have failed due to lack of specificity. Antibodies have faced a similar situation since there is a tradeoff between selectivity and potency due to the antibody binding to a specific (open or close) conformation of the channel. Indeed, even commercially available antibodies targeting the human channel are poor for a western blot. Interference RNA (RNAi) has also been utilized to target Nav1.7. As an exogenous system, however, RNAi competes with endogenous machinery such as microRNA or RISC complex function. Thus, RNAi can compete with and impair fundamental homeostatic mechanisms of RNA synthesis and degradation. In addition, due to high RNA turnover, RNAi methods have poorer pharmacokinetics prospects and require higher dosage. It is mainly due to these drawbacks that none of the Nav1.7-targeting treatments based on these methods have yet succeed to reach the final phase of clinical trials. In contrast, disclosed here is the use of nucleic acid binding domains (e.g., Zinc-Finger proteins) and epigenetic modulators (e.g., KRAB repressor) to repress the transcription of SCN9A, and/or other genes associated with pain. As permanent ablation of pain is not desired, there is no permanent genome editing using such methods. Instead, these epigenomic engineering methods enable transient modulation of Na_{V}1.7 gene expression. Additionally, this approach may have lower risk of off-target effects than other approaches. Rather than pharmacologically targeting the protein or RNA, this approach targets Na_{V}1.7 at the DNA level. This may result in longer lasting results than methods targeting protein or RNA. With this approach, one can engineer highly specific, long-lasting, and reversible treatments for pain. Treatment duration is important because many pain states resulting from chronic inflammation and nerve injury are enduring conditions which typically require continual re-medication. This genetic approach provides ongoing, controllable regulation of the aberrant pain processing. Further, since the disclosed approach can be easily designed to target several genes, it represents a new paradigm in pain management since it provides a synergistic way of targeting single or multiple sodium channels for more potent pain relief.

### Methods of Treatment

Various embodiments provide for methods of treating a disease or condition in a subject with the compositions described herein. In some embodiments, the composition comprises an epigenetic modulator or a polynucleotide encoding an epigenetic modulator. The composition may be delivered via AAV or non-viral vehicles.

In example embodiments, the disease or condition comprises pain. Pain includes neuropathic, inflammatory, visceral, migraine, erythromelalgia, fibromyalgia, idiopathic and somatic pain. In some embodiments, the pain is chemotherapy-induced (e.g., paclitaxel-induced). Inflammatory pain comprises rheumatoid arthritis pain. The disease or condition also includes those where Na_{V}1.7 or other genes involved in pain could be targeted. In some embodiments, the disease or condition is associated with Na_{V}1.7. In some embodiments, a method of treatment comprises treating inflammation in the subject with a composition comprising an epigenetic modulator or a polynucleotide encoding an epigenetic modulator. The inflammation may be associated with a disease or condition, such as arthritis.

In some embodiments, the disease or condition is cancer. In some embodiments, the disease or condition includes small-fiber neuropathy, back pain, rheumatoid arthritis, osteoarthritis, spinal stenosis, chronic cough, migraine, trigeminal neuralgia, erythromelalgia, and paroxysmal extreme pain disorder.

In some embodiments, the disease or condition comprises a channelopathy. Channelopathies include Dravet syndrome, Epilepsy syndromes, Familial hemiplegic migraine, Ohtahara syndrome, West syndrome, Lennox-Gastaut syndrome, sodium channel myotonia, autism, Long QT syndrome, Brugada syndrome, Progressive cardiac conduction disease (also called Lenègre disease), and the like. In some embodiments, the condition may be pain, inflammation, or both.

In some embodiments, the disease or condition comprises a neurological disease or condition. Neurological diseases or conditions include dementia, Alzheimer's disease, Parkinson disease, Huntington's disease, schizophrenia, Amyotrophic lateral sclerosis (ALS) and Multiple Sclerosis. The disease or condition may comprise a central nervous system ailment.

In some embodiments, the disease or condition comprises an inflammatory disease or condition. As a non-limiting example, the inflammatory disease or condition is rheumatoid arthritis.

In some embodiments, the disease or condition comprises an infection.

In some embodiments, the disease or condition comprises Beta-thalassemia, Fragile X, centronuclear myopathy, Prion disease, Angelman Syndrome, Lafora disease, or Alexander disease, or a combination thereof.

In some embodiments, a method of treatment comprises administering a nucleic acid composition described herein and one or more additional active agents. For instance, the additional active agent may be used to complementarily treat the disease or condition.

In some embodiments, a subject refers to any animal, including, but not limited to, humans, non-human primates, rodents, and domestic and game animals. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a human.

In various embodiments, a subject can be one who has been previously diagnosed with or identified as suffering from or having a disease or condition in need of treatment. In various embodiments, the subject previously diagnosed with or identified as suffering from or having the disease or condition may or may not have undergone treatment for a condition. In other embodiments, a subject can also be one who has not been previously diagnosed as having a disease or condition and the therapeutic is used for prevention (prophylactically).

### Pharmaceutical Compositions, Administration and Dosage

In various embodiments, the compositions herein are formulated for delivery via any route of administration. “Route of administration” may refer to any administration pathway known in the art, including but not limited to intrathecal, epidural, intravenous, transdermal, intranasal, oral, mucosal, or other delivery methods, and/or via single or multiple doses. Example routes of administration for the nucleic acids described herein include lumbar intrathecal puncture, intracisternal magna administration, and intraganglionic administration.

In an example embodiment, the composition is delivered into the spinal intrathecal space using any appropriate delivery method. This approach may be particularly useful when targeting Na_{V}1.7 because the role played by Na_{V}1.7 is in the nociceptive afferents, and their cell bodies are in the respective segmental dorsal root ganglion (DRG) neurons. Therefore, delivery to the spinal intrathecal space may efficiently deliver compositions targeting Na_{V}1.7 to the DRG neurons, which can minimize the possibility of off target biodistribution and reduce viral load required for transduction.

It is appreciated that actual dosage can vary depending on the route of administration, the delivery system used (e.g., AAV or liposome, etc), the target cell, organ, or tissue, the subject, as well as the degree of effect sought. Size and weight of the tissue, organ, and/or patient can also affect dosing. Doses may further include additional agents, including but not limited to a carrier. Non-limiting examples of suitable carriers are known in the art: for example, water, saline, ethanol, glycerol, lactose, sucrose, dextran, agar, pectin, plant-derived oils, phosphate-buffered saline, and/or diluents. The pharmaceutical compositions can also contain any pharmaceutically acceptable carrier.

The pharmaceutical compositions may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of nucleic acid (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. When the intrathecal route is recommended, the pharmaceutical product may be diluted ex vivo with the subject cerebrospinal fluid prior to administration to achieve an isobaric solution.

### Kits

Further provided is a kit to perform methods described herein. The kit is an assemblage of components, including at least one of the compositions described herein. Thus, in some embodiments, the kit comprises a nucleic acid encoding a nucleic acid binding domain and an epigenetic modulator. The nucleic acid may be combined with, or complexed to, another component such as a vehicle for delivery, or may be unmodified for direct delivery. In some cases, the nucleic acid is complexed with a cationic molecule. In some cases, the nucleic acid is configured for delivery via a viral delivery vehicle such as an AAV capsid protein.

Instructions for use of the components may be included in the kit. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of a composition containing a nucleic acid herein. The packaging material generally has an external label which indicates the contents and/or purpose of the kit and/or its components.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

As used herein, the term percent “identity,” in the context of two or more nucleic acid or polypeptide sequences, may refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity may be performed using the BLAST algorithm, which is described in Altschul et al. (J. Mol. Biol. 215:403-410 (1990)). Software for performing BLAST analyses is publicly available through the National Center for Biotechnology Information.

A composition can be administered alone or in combination with other treatments, either simultaneously or sequentially dependent upon the condition to be treated.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the joints (intraarticular), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 21^{st} Ed., Mack Publ. Co., Easton, Pa. (2005), incorporated herein by reference in its entirety.

### EXAMPLES

The invention is further illustrated by the following non-limiting examples.

### Example 1

### AAV9 Vectors Encoding Na_{V}1.7 Epigenetic Modulators

This example describes AAV9 vectors encoding Na_{V}1.7 epigenetic modulators. A payload polynucleotide encoding an epigenetic modulator under transcriptional control of a promoter is encapsulated by an AAV9 capsid. The epigenetic modulator, when expressed from the payload polynucleotide, modulates Na_{V}1.7 expression. The Na_{V}1.7 epigenetic modulator comprises a zinc figure protein targeting Na_{V}1.7 (e.g., any of SEQ ID NO: 1-SEQ ID NO: 4 or SEQ ID NO: 99-SEQ ID NO: 169) linked to a repressor domain (e.g., any of SEQ ID NO: 5-SEQ ID NO: 20). Optionally, the epigenetic modulator comprises a sequence of any one of SEQ ID NO: 25-SEQ ID NO: 36. The promoter is a Na_{V}1.7-specific promoter (e.g., any of SEQ ID NO: 37-SEQ ID NO: 43), a neuron-specific promoter (e.g., a human synapsin promoter of SEQ ID NO: 44), or a ubiquitous promoter (e.g., any of SEQ ID NO: 45-SEQ ID NO: 47). The promoter is SEQ ID NO: 98.

### Example 2

### Treatment of Pain by Epigenetic Modulation of Na_{V}1.7

This example describes treatment of pain by epigenetic modulation of Na_{V}1.7. A vector encoding an epigenetic modulator of any one of SEQ ID NO: 25-SEQ ID NO: 36 is administered to a subject in pain or having inflammation. Optionally, the vector is an AAV9 vector described in EXAMPLE 1. Following administration, the epigenetic modulator is expressed in the subject and represses expression of Na_{V}1.7 in the subject. The pain, inflammation, or both is reduced in the subject.

### Example 3

### In Vitro Human Optimization of Zinc Finger Designs Targeting the Human NaV1.7

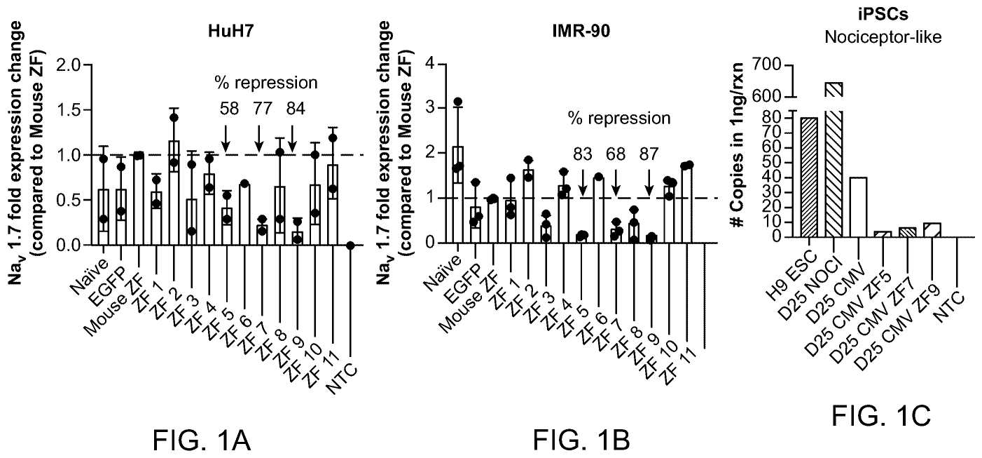

This example describes a repression of SCN9A in human cell lines to identify the lead zinc finger candidate. In order to design a zinc finger targeting the human SCN9A (Nav1.7) DNA sequence, new ZF constructs that bind the human SCN9A DNA sequence were designed and tested in vitro to determine which has the highest target engagement. For genome repression, we and others have found that targeting close to the TSS (−50 to +300 bp relative to the TSS) increases efficacy (Gilbert et al., 2013). This constraint complicates the design but on the other hand reduces the potential of off-targets, as DNA-binding does not necessary translate in gene modulation. Thus, we designed eleven new ZF constructs that recognize the human SCN9A gene and tested them in vitro to determine which one shows higher efficacy in target engagement, in a similar strategy as shown before with Neuro2a mouse cells (FIG. 1). We chose HuH7 (JCRB0403; JCRB Cell Bank) and IMR-90 (ATCC® CCL-186™) for initial screening given their high NaV1.7 expression levels. The National Center for Advancing Translational Sciences (NCATS), a branch of the NIH, validated our best ZF candidates (ZF5, ZF7 and ZF9) in their induced pluripotent stem cells (iPSCs) derived to nociceptor-like cells as part of an ongoing collaboration for the helping to end addiction long-term (HEAL) initiative. Eleven zinc finger proteins were transfected into HuH7 cells and the level of NaV1.7 repression was measured through qPCR using the ΔΔCt method (FIG. 1A). The same ZF arrays were tested in IMR-90 and the repression levels were measured through qPCR using the ΔΔCt method (FIG. 1B). Data obtained by the NCATS (branch of the NIH) with our lead ZF candidates (FIG. 1C). Repression levels obtained through ddPCR.

### Example 4

### Isolation of the NaV1.7 Specific Promoter

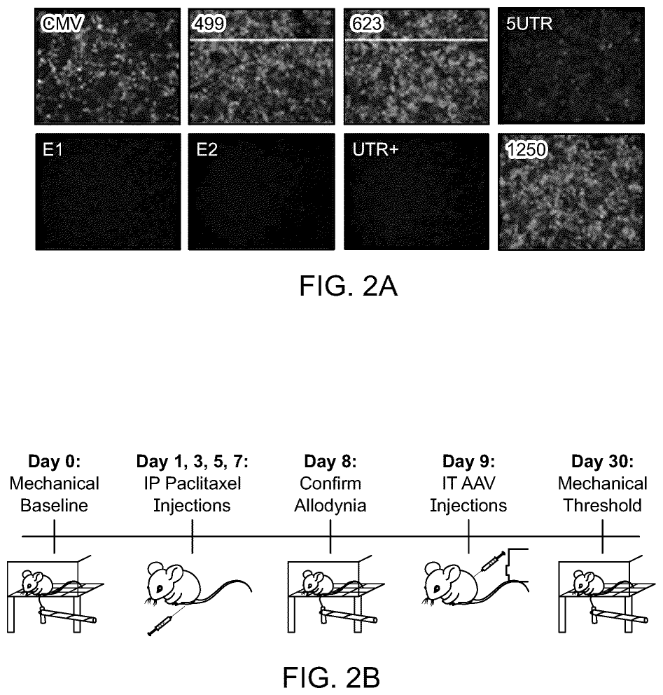

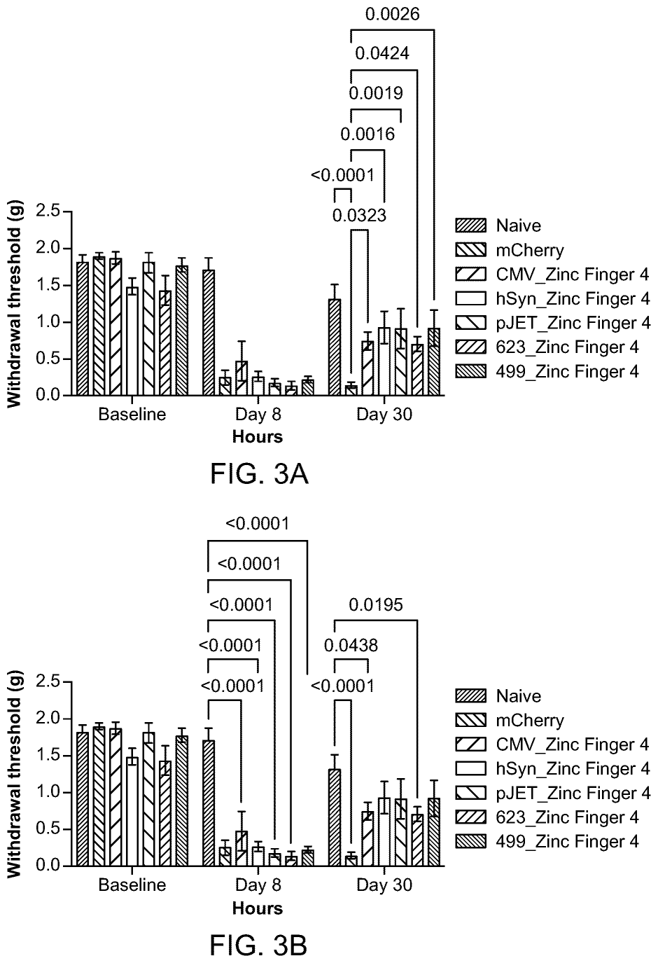

This example describes an in vivo promoter study using a chemotherapy-induced peripheral neuropathy (CIPN) model to determine the NaV1.7 specific promoter. In order to reduce potential toxicity, we propose the use of a minimal specific promoter that would drive the expression of the ZF only in NaV1.7-expressing cells. Potentially, with a NaV1.7 specific promoter, the ZF effectors would have increased expression when the external conditions increase NaV1.7 expression (which has been described in multiple pain conditions), and thus, the dose levels may modulate themselves as required by the environment. Thus, we studied several DNA regions around the TSS of human SCN9A and tested the expression of the fluorescence protein mCherry in cells that express NaV1.7 (HuH7; FIG. 2A). As a negative control we used the non-NaV1.7-expressing cell line MCF-7 to determine there was no “leaky” expression. Then, we tested the preclinical in vivo efficacy using the CIPN model of pain (FIGS. 2B, 3A and 3B). In this case, we not only used the best two Na_{V}1.7 promoters from the in vitro work, 499 and 623, but also a weak synthetic promoter, pJET, that has previously been used with AAV9 delivered intrathecally (Bailey et al., 2018), and the human synapsin 1 promoter that drives expression only in neurons (Schoch et al., 1996; McLean et al., 2014; Jackson et al., 2016). Even though 623 promoter presented slightly slower efficacy than the rest of the promoters, 499 promoter presented as much efficacy as the rest of the promoters. We therefore selected the 499 promoter sequence moving forward.

### Example 5

### Analysis of Zinc Finger Protein Profile in Inherited Erythromelalgia Patient

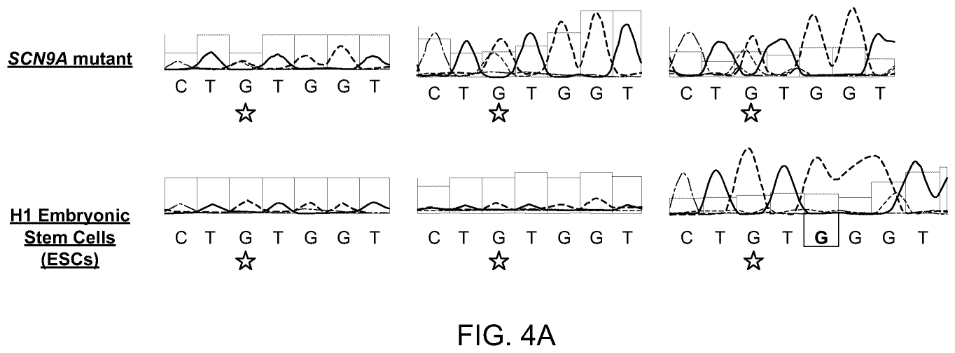

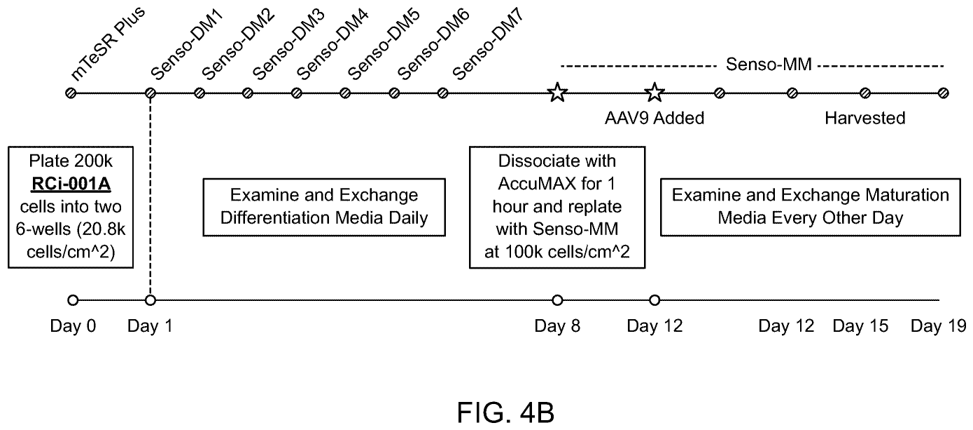

This example describes the zinc finger profile in a patient with inherited erythromelalgia. Zinc finger protein profiles in induced pluripotent stem cells (iPSCs) derived from patients diagnosed with inherited erythromelalgia (IEM) due to SCN9A gene mutations were determined. Genotyping was performed on the 1197th bp of the SCN9A mutant iPSC line and a healthy H1 embryonic stem cell (ESC) line with a wildtype SCN9A genomic sequence. The SCN9A mutant iPSC line had a consistent conflict between guanine and adenine bases, but the ESC line recalled guanine without conflict, as depicted in FIG. 4A. The results indicate a heterozygous guanine to alanine point mutation in the SCN9A mutant iPSC line at the 1197th bp of the open reading frame. This heterozygous guanine to alanine point mutation results in a conversion of the 400th amino acid of SCN9A, which converts Valine to Methionine (Cao, Lishuang et al. Science translational medicine, vol. 8,335 (2016): 335ra56. doi: 10.1126/scitranslmed.aad7653).

Differentiation of iPSCs Derived from Patients with IEM (IEM-PSCs)

iPSCs derived from patients with IEM were differentiated based on the experimental scheme depicted in FIG. 4B, in which sensory neuronal-like cells with SCN9A expression were generated. iPSCs were differentiated into premature sensory-like neurons by receiving seven daily media changes, each having a unique mixture of differentiation-inducing factors. On Day 8 of the differentiation process, the cells were split and replated at 100,000 cells per cm² of surface area. From days 8 to 19, the cells were maintained in the maturation media, Senso-MM, and the media was changed twice a day. On day 12, the cells were transduced for 48 hours with various titers of AAV9 and harvested on day 19.

### Confirmation of Differentiation of IEM-PSCs

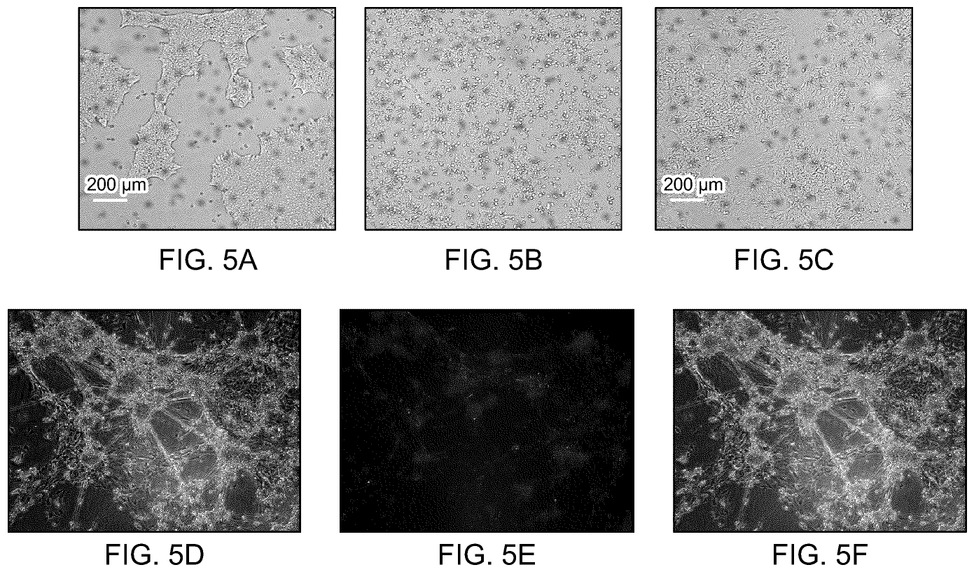

The sensory neuronal differentiation process for iPSCs lasted 19 days. The iPSCs were detached and replated on day 7 of the differentiation process. On day 9 of the differentiation process, iPSCs were transduced with AAV9 encoding mCherry control or SCN9A-repressing zinc finger candidates. Confocal images of iPSC-derived sensory neurons from patients diagnosed with IEM are depicted in FIGS. 5A-F. The sensory neuronal differentiation of IEM-iPSCs on days 0, 2, and 6 is shown in FIGS. 5A, 5B, and 5C, respectively. FIG. 5D shows the brightfield channel (control), FIG. 5E shows the mCherry channel (control), and FIG. 5F shows an overlay of the brightfield and mCherry channels (control).

### Repression of Nav 1.7 in IEM-iPSCs

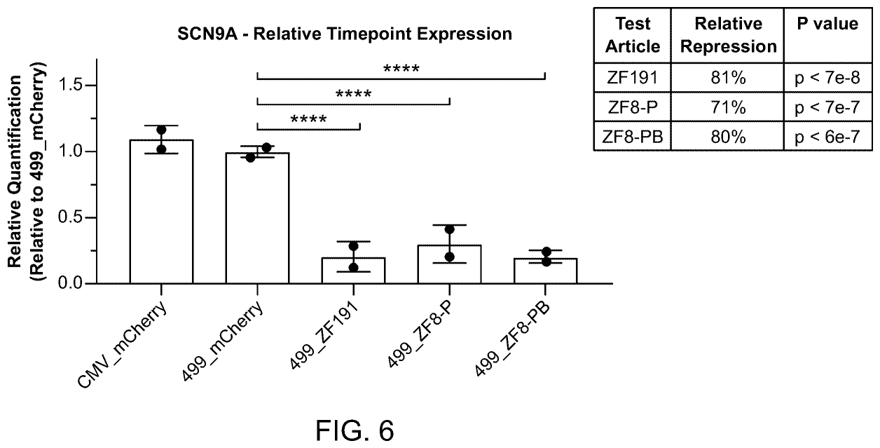

The expression levels of Nav1.7 in the presence of various zinc finger proteins targeting SCN9A (ZF191, ZF8-P, and ZF8-PB) were determined by qPCR. All zinc finger proteins targeting SCN9A significantly repressed the expression levels of Nav1.7, as shown in FIG. 6.

### Example 6

### Nav1.7 Downregulation Using Human Dorsal Root Ganglia (DRG) Ex Vivo Cultures Optimization of AA9 Transduction of Human DRG

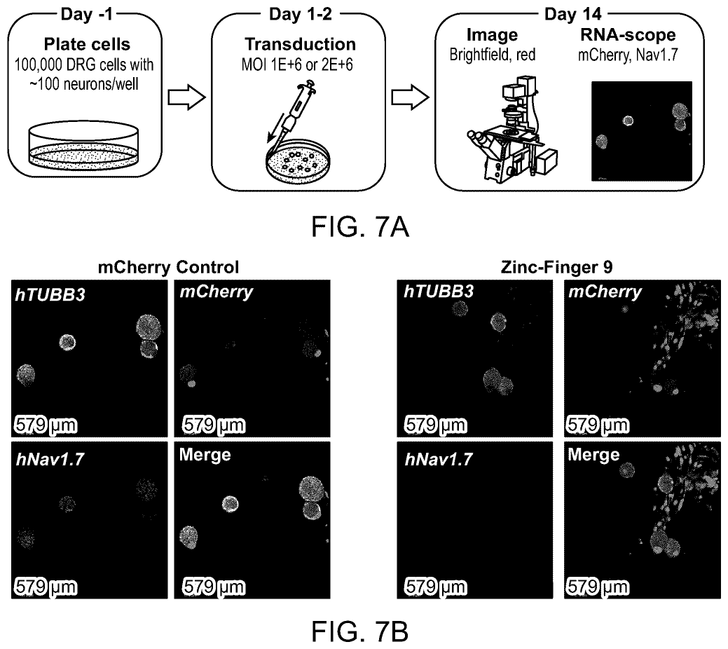

This example describes a downregulation of Nav1.7 using human DRG ex vivo cultures. In order to investigate the downregulation of Nav1.7, human dorsal root ganglia (DRG) were transduced with AAV9 ex vivo at different multiplicities of infection (MOIs) of AAV9 for optimization of transduction, as depicted in FIG. 7A. Human DRG cells were plated at a density of 100,000 cells per well, comprising approximately 100 neurons. After 2 to 3 days of plating, the human DRG cells were transduced with various MOIs of AAV9 (e.g., MOI of 1E+6, MOI of 2E+6, etc.). RNAscope was performed to determine the gene expression level of Nav1.7 at 12 to 13 days after the transduction of human DRG with AAV9. The number of Nav1.7 mRNA transcripts, as determined by RNAscope, was based on the fluorescent spot count from approximately 100 randomly selected cells in microscopic images. The per-cell copy number was determined by dividing the total spot count by the number of cells counted. Representative RNAscope images are shown in FIG. 7B.

### RNAscope Quantification of SCN9A in Ex Vivo Human DRG

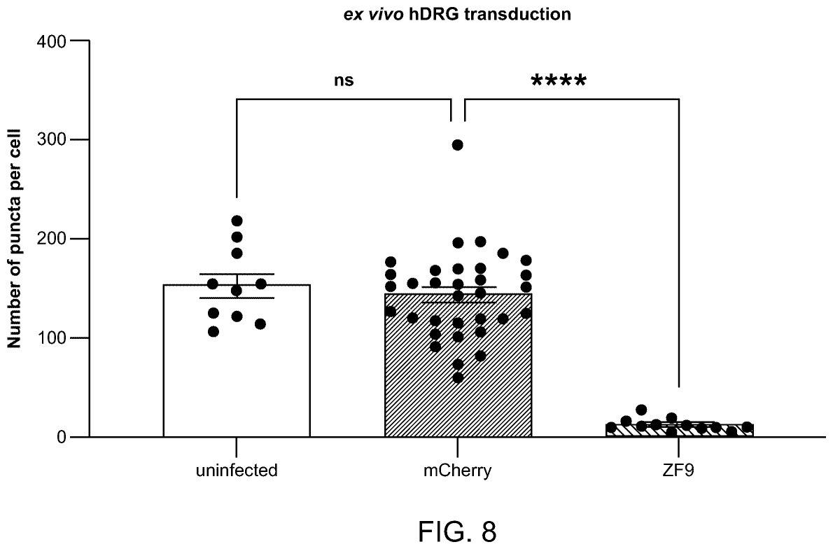

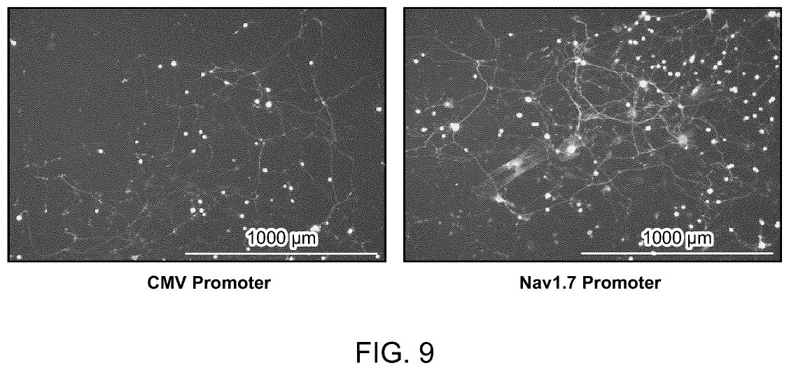

The RNAscope quantification of SCN9A gene expression levels was performed. The results suggest that the zinc finger protein ZF9 significantly repressed the expression of SCN9A ex vivo. Human DRG cultures treated with ZF9 showed a 91% repression of SCN9A gene expression compared to the human DRG cultures with mCherry (negative control), as depicted in FIG. 8.

### Efficacy of Nav1.7-Specific Promoter in Transgene Expression in Mice DRG Ex Vivo Cultures

The transgene expressions in ex vivo cultures of mouse DRG with either a Nav1.7-specific promoter or a CMV promoter were measured. CMV and Nav1.7-specific promoters led to strong mCherry expressions in ex vivo cultures following AAV9-mCherry transduction, as shown in FIG. 9.

### AAV9 Nav1.7-1 Transduction of Whole DRG

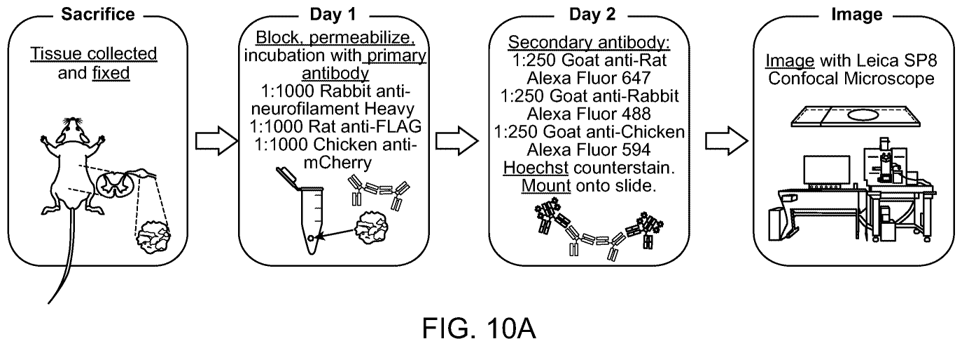

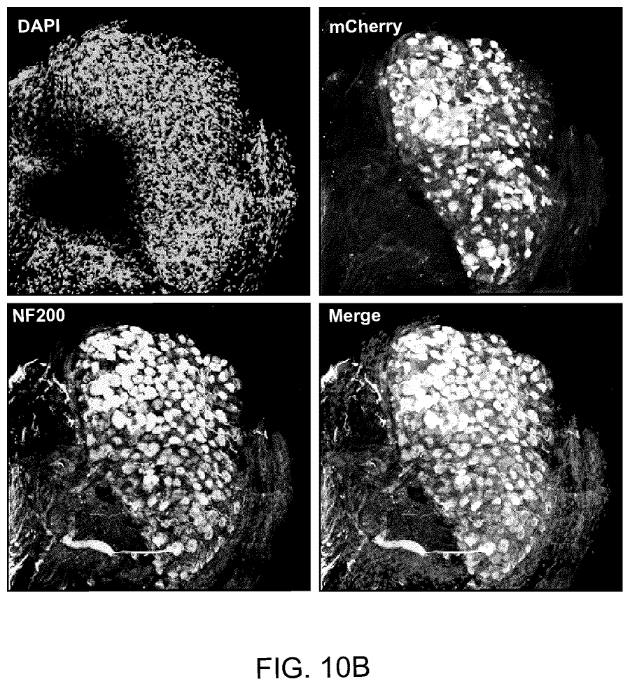

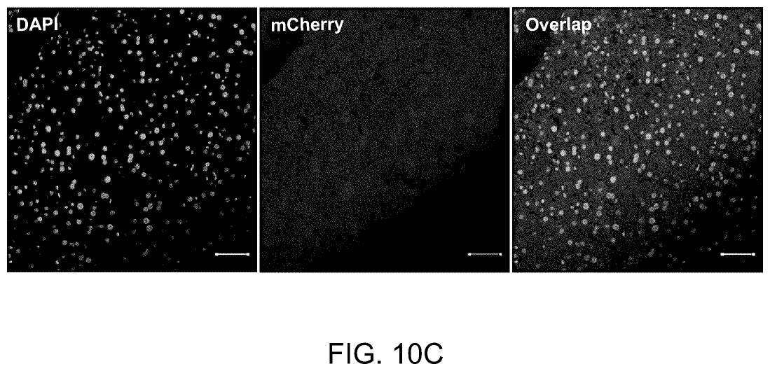

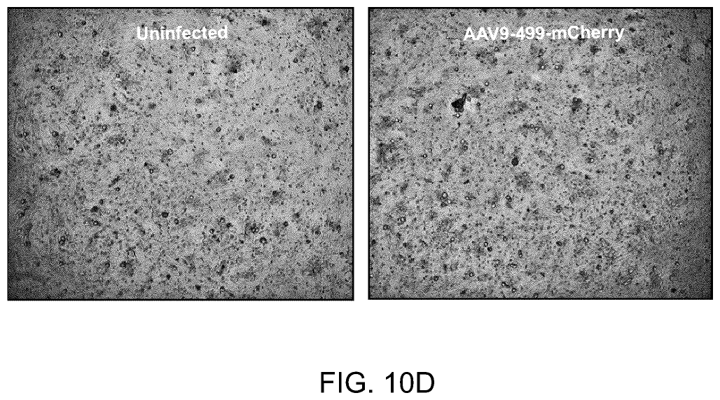

The transduction of whole DRG with AAV9 Nav1.7-1 was assessed using confocal imaging. A schematic method of the whole mouse DRG immunofluorescence is depicted in FIG. 10A. Mice were administered 7.5×10¹⁰ vector genomes (vg) per mouse via intrathecal (IT) injection. After harvesting, tissues were collected and fixed for a day. Following blocking and permeabilization, the tissues were incubated with primary antibodies (rabbit anti-neurofilament heavy antibody, rat anti-FLAG, and chicken anti-mCherry) at a concentration of 1:1000. Subsequently, tissues were incubated with secondary antibodies (goat anti-rat Alexa Fluor 647, goat anti-rabbit Alexa Fluor 488, and goat anti-chicken Alexa Fluor 594) at a concentration of 1:250. Following this, tissues were stained with Hoechst for nuclear counterstaining and then mounted on slides for image analysis using a confocal microscope (Leica SP8). As shown in FIG. 10B, the Nav1.7-1 promoter induced mCherry expression in mice. Immunohistochemistry (IHC) performed on the liver of the mice showed that the Nav1.7-specific promoter did not result in transgene expression, as shown in FIG. 10C. Ex vivo human DRG cultures infected with AAV-499-mCherry resulted in transgene expression, as shown in FIG. 10D.

### Example 7

### Efficacy of ZF4-KRAB Delivered Via AAV9 in Target Engagement and Pain Amelioration

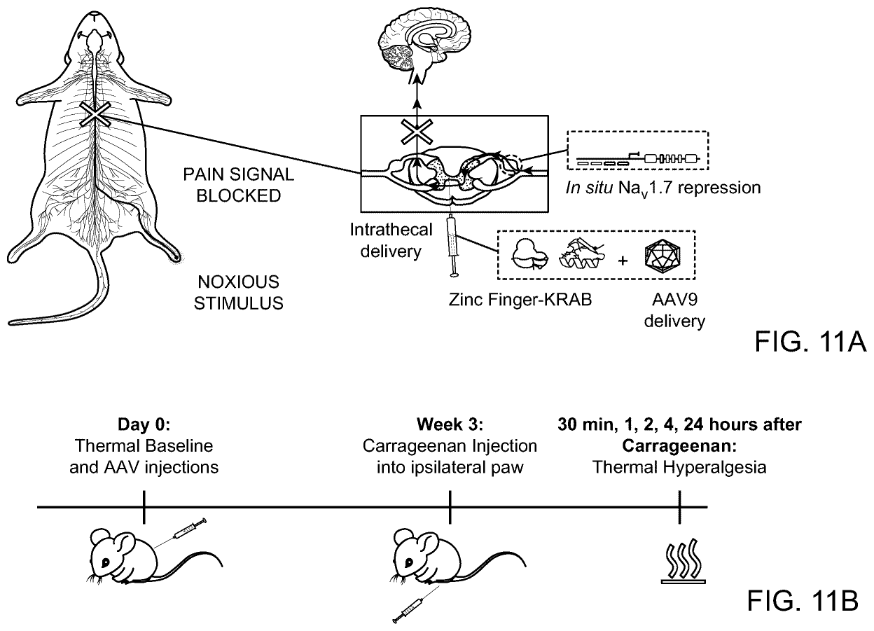

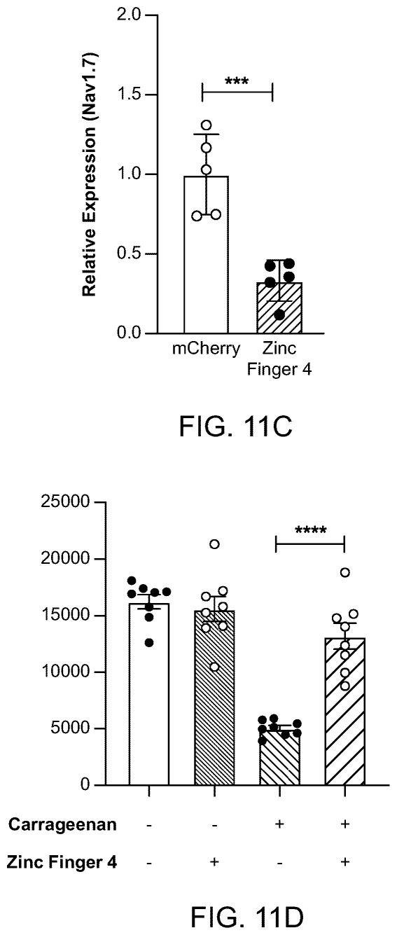

This example describes a chemotherapy-induced neuropathic pain model. After having established in vivo efficacy in an inflammatory pain model, the epigenome repression strategy for neuropathic pain using a polyneuropathy model by the chemotherapeutic paclitaxel is tested. To establish this model, mice were first injected with 1×10¹² vg/mouse of AAV9-mCherry (n=8), AAV9-ZF4-KRAB (n=8), or saline (n=16). 14 days later and before paclitaxel administration, the baseline for tactile threshold was established (von Frey filaments). Mice were then administered paclitaxel at days 14, 16, 18, and 20, with a dosage of 8 mg/kg (total cumulative dosage of 32 mg/kg), with a group of saline injected mice not receiving any paclitaxel (n=8) to establish the tactile allodynia caused by the chemotherapeutic, as illustrated in FIG. 11A. At 21 and 105 days after, mice were tested for tactile allodynia via von Frey filaments, with one group of saline-injected mice (n=8) injected with intraperitoneal gabapentin (100 mg/kg) 1 hour before testing, as depicted in FIG. 11B and FIG. 11C. A 50% tactile threshold was calculated. A significant decrease in tactile allodynia was observed for mice injected with AAV9-Zinc-Finger-4-KRAB at 21 (P=0.0007) and 105 (P<0.0001) days after AAV9 injections. Additionally, compared to the 21 day timepoint, mice from the AAV9-mCherry (n=8) group showed increased tactile allodynia at day 105 as compared to day 21 and responded to the lowest von Frey filament examined (0.04 g). In comparison, mice receiving AAV9-Zinc-Finger-4-KRAB had increased withdrawal thresholds, indicating that in situ NaV1.7 repression leads to long-term prevention in chemotherapy-induced tactile allodynia. Additionally, an increase in the number of withdrawal responses is seen in mice tested for cold allodynia in the AAV9-mCherry negative control group), while AAV9-Zinc-Finger-4-KRAB groups had a decrease in withdrawal responses (P<0.0001 for both day 21 and 105), indicating that in situ repression of NaV1.7 also leads to long-term prevention of chemotherapy-induced cold allodynia (FIGS. 11B, 11C). The key for success of this therapeutic approach is the duration of the effect (or half-life of the therapy) to avoid frequent lumbar punctures and the results indicate that the therapeutic approach is long-lasting (pain can be prevented for at least 44 weeks according to the inflammatory pain model and at least 15 weeks in the CIPN model) (FIGS. 11D and 11E).

### In Vivo Efficacy of ZF4-KRAB in a CIPN Model

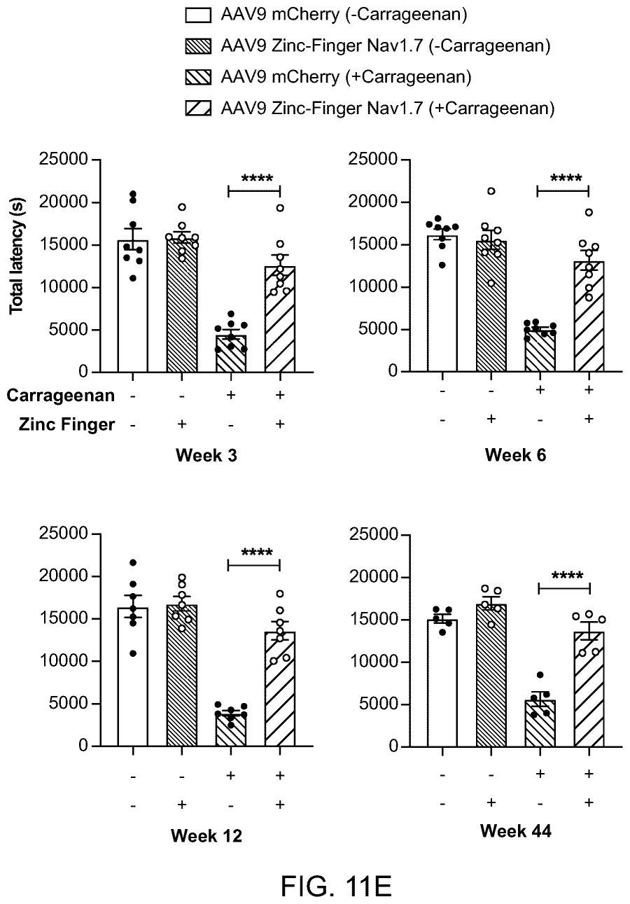

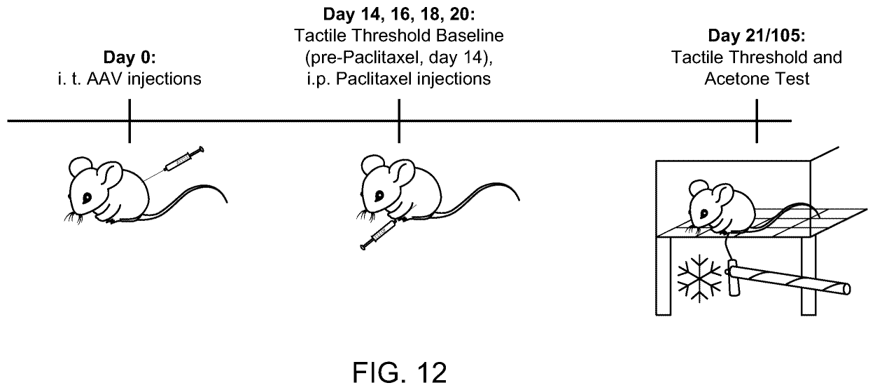

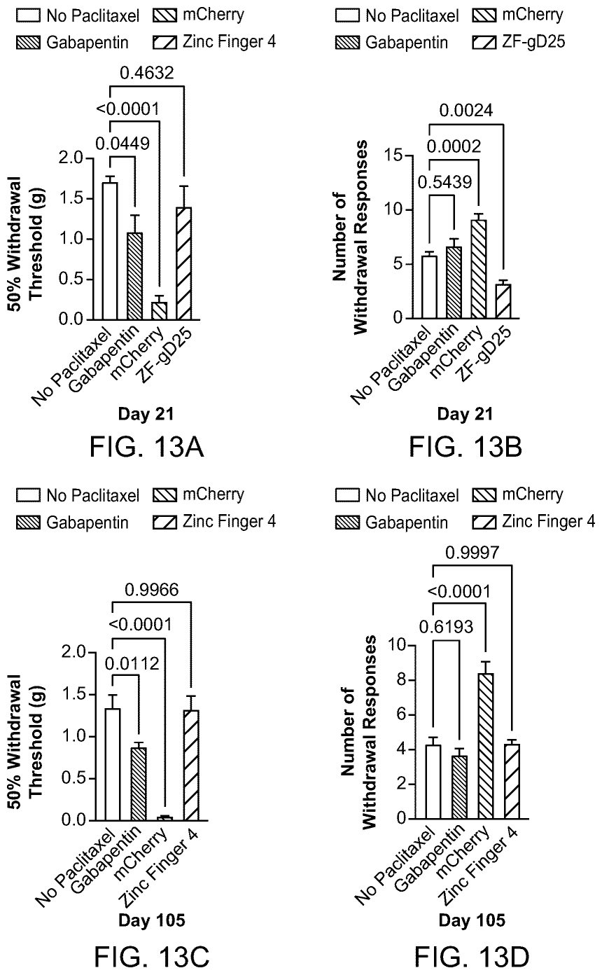

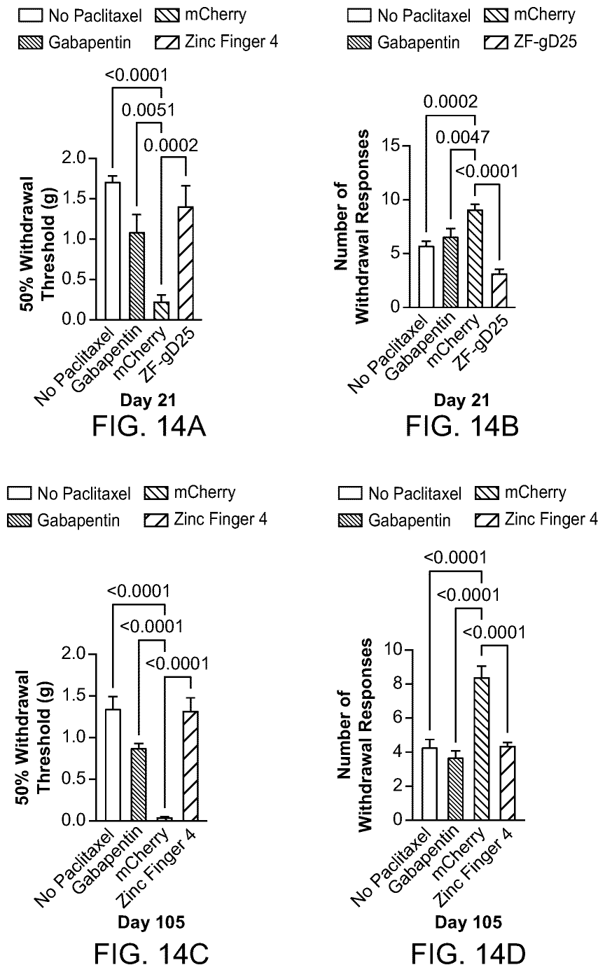

The reverse of pain after creating an allodynic state with the chemotherapy induced peripheral neuropathy (CIPN) mouse model was determined. As shown in FIG. 12, the downregulation of NaV1.7 with ZF4-KRAB can reverse the allodynia produced by a chemotherapy cycle of paclitaxel. A schematic of the paclitaxel-induced neuropathic pain model is depicted in FIG. 11A. Mice were administered with AAV9-mCherry via intrathecal (IT) injection, AAV9-ZF4-KRAB, or saline. Following baseline von Frey threshold testing at day 14, mice were then injected intraperitoneally (i.p.) with 8 mg/kg of paclitaxel at 14, 16, 18, 20 days after IT injection (32 mg/kg cumulative dosage). 21 (FIGS. 13A, 13B, 14A, and 14B) and 105 (FIGS. 13C, 13D, 14C, and 14D) days after IT injection, mice were tested for tactile allodynia via von Frey filaments and for cold allodynia via the application of acetone. ZF4-KRAB reduces paclitaxel-induced tactile allodynia compared to controls (FIG. 13A; FIG. 14A). ZF4-KRAB reduces paclitaxel-induced cold allodynia compared to controls (FIG. 13B; FIG. 14B). ZF4-KRAB reduces paclitaxel-induced tactile allodynia 105 days after last paclitaxel injection compared to controls (FIG. 13C; FIG. 14C). ZF4-KRAB reduces paclitaxel-induced cold allodynia compared to controls (FIG. 13D; FIG. 14D). FIG. 13 indicates statistical analysis compared to the naïve group (No Paclitaxel), and FIG. 14 shows statistical analysis compared to the mCherry group. Similar data was obtained using a CRISPR-dCas9 system (data not shown).

### Safety Assessment in Mice

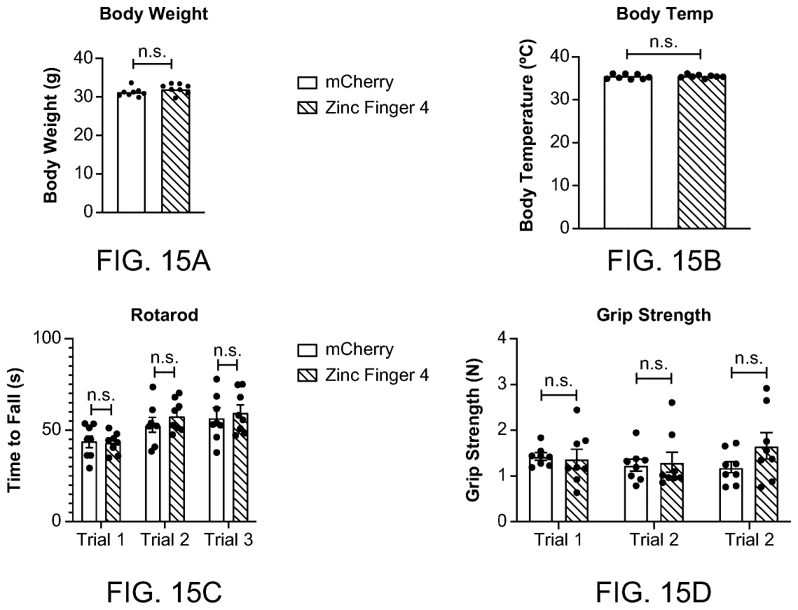

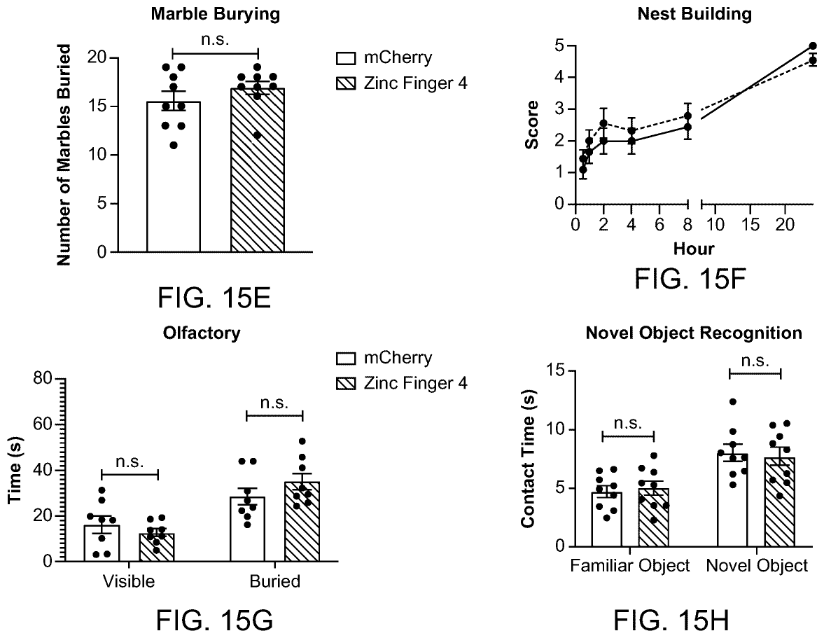

Initial safety assessments were determined in mice. To determine potential safety side effects of NaV1.7 epigenetic repression via ZF-KRAB, we performed a series of toxicity/side effect test battery for examination of general health and behaviors in mice. These tests are sensitive to changes in self-care, increases in distress/stress, and illness. We injected mice IT with 1×10¹² vg/mouse of AAV9-mCherry (n=8) or AAV9-ZF4-KRAB (n=8). We then examined the mice 8-12 weeks after IT injection for body weight and body temperature (FIGS. 15A and 15B). AAV9-ZF4-KRAB did not produce indices of dysfunction. To determine whether there was any change in motor function, we performed a rotarod balancing test, which requires a variety of proprioceptive, vestibular, and fine-tuned motor abilities as well as motor learning capabilities (Carter et al., 2001). Mice were placed on a rotating rod and subjected to an accelerating test strategy, whereby the rod starts at 0 rpm and then accelerates at 10 rpm (Roberts et al., 1993; Finn et al., 1997). The time to fall (seconds) was recorded and mice were tested in 3 sets of 3 trials, and we found no significant changes in the time to fall compared to the controls (FIG. 15C). We also measured grip strength, marble burying, and nest building, but found no significant changes in AAV9-ZF4-KRAB-injected mice (FIGS. 15D-F). Importantly, as loss-of-function NaV1.7 mutations in individuals with CIP have anosmia (Cox et al., 2006; Weiss et al., 2011), we performed an olfactory test, which examines the ability of the mice to locate a desired food item buried under bedding. To determine whether mice had a loss of smell, we performed a test that examines the ability of a mouse to locate a desired food item buried under bedding. We found no significant changes in olfaction for the AAV9-ZF4-KRAB injected mice (FIG. 15G), which probably indicates AAV9 did not transduce neurons of the olfactory bulb. Lastly, we performed a cognitive test, to determine whether any cognitive side effects were seen using a novel object recognition test. This test assays recognition memory while leaving the spatial location of the objects intact and is believed to involve the hippocampus, perirhinal cortex, and raphe nuclei (Winters et al., 2004; Mumby et al., 2005; Lieben et al., 2006). The basic principle is that animals explore novel environments and that with repeated exposure decreased exploration ensues (Berlyne, 1950) (i.e., habituation). We found no significant changes in cognition for the AAV9-ZF4-KRAB injected mice (FIG. 15H). The findings suggest that Nav1.7 epigenetic repression via Zinc-Fingers have no general effects upon non-nocifensive behaviors. Overall, ZF4-KRAB shows safety and efficacy for treating chronic inflammatory and neuropathic pain in mice.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A zinc finger protein comprising:
(a) at least 90% sequence identity to SEQ ID NO: 2;
(b) at least 98% sequence identity to SEQ ID NO: 3; or
(c) at least 90% sequence identity to SEQ ID NO: 4.

2. The zinc finger protein of claim 1, comprising at least 95% sequence identity to SEQ ID NO: 2 or SEQ ID NO: 4.

3. The zinc finger protein of claim 1, comprising at least 97% sequence identity to SEQ ID NO: 2 or SEQ ID NO: 4.

4. The zinc finger protein of claim 1, comprising a sequence of any one of SEQ ID NO: 1-SEQ ID NO: 4.

5. The zinc finger protein of any one of claims 1-4, wherein the zinc finger protein has affinity for SCN9A.

6. The zinc finger protein of any one of claims 1-5, wherein the zinc finger protein has affinity for a polynucleotide sequence having at least 90% sequence identity to any one of SEQ ID NO: 73-SEQ ID NO: 97.

7. The zinc finger protein of any one of claims 1-6, wherein the zinc finger protein has affinity for a polynucleotide sequence of any one of SEQ ID NO: 73-SEQ ID NO: 97.

8. An epigenetic modulator comprising the zinc finger protein of any one of claims 1-7 linked to a repressor domain.

9. An epigenetic modulator comprising a zinc finger protein linked to a repressor domain, wherein the zinc finger protein comprises at least 90% sequence identity to any one of SEQ ID NO: 1-SEQ ID NO: 4 or SEQ ID NO: 99-SEQ ID NO: 169.

10. The epigenetic modulator of claim 9, wherein the zinc finger protein comprises at least 95%, at least 97%, at least 98%, or 99% sequence identity to any one of SEQ ID NO: 1-SEQ ID NO: 4 or SEQ ID NO: 99-SEQ ID NO: 169.

11. The epigenetic modulator of claim 9 or claim 10, wherein the zinc finger protein comprises any one of SEQ ID NO: 1-SEQ ID NO: 4 or SEQ ID NO: 99-SEQ ID NO: 169.

12. The epigenetic modulator of any one of claims 8-11, wherein the zinc finger protein has affinity for a SCNA9.

13. The epigenetic modulator of any one of claims 8-12, wherein the zinc finger protein has affinity for a polynucleotide sequence having at least 90% sequence identity to any one of SEQ ID NO: 73-SEQ ID NO: 97.

14. The epigenetic modulator of any one of claims 8-13, wherein the zinc finger protein has affinity for a polynucleotide sequence of any one of SEQ ID NO: 73-SEQ ID NO: 97.

15. The epigenetic modulator of any one of claims 8-14, wherein the repressor domain comprises ZIM3, SID, KOX1, ZNF554, ZNF264, ZNF324, MeCP2, MBD2b, SID, HPla, SIRT5, SETD8, HDT1, SUPR, FOG1, DNMT3A, DNMT3L, DMT3, or a combination thereof.

16. The epigenetic modulator of any one of claims 8-15, wherein the repressor domain comprises at least 90% sequence identity to any one of SEQ ID NO: 5-SEQ ID NO: 20.

17. The epigenetic modulator of any one of claims 8-16, wherein the repressor domain comprises any one of SEQ ID NO: 5-SEQ ID NO: 20.

18. The epigenetic modulator of any one of claims 8-17, wherein the repressor domain comprises SEQ ID NO: 5 or SEQ ID NO: 9.

19. The epigenetic modulator of any one of claims 8-18, wherein the zinc finger protein is linked to the repressor domain via a peptide linker.

20. The epigenetic modulator of any one of claims 8-19, further comprising a second repressor domain.

21. The epigenetic modulator of claim 20, wherein the second repressor domain comprises ZIM3, SID, KOX1, ZNF554, ZNF264, ZNF324, MeCP2, MBD2b, SID, HP1a, SIRT5, SETD8, HDT1, SUPR, FOG1, DNMT3A, DNMT3L, DMT3, or a combination thereof.

22. The epigenetic modulator of claim 20 or claim 21, wherein the second repressor domain comprises at least 90% sequence identity to any one of SEQ ID NO: 5-SEQ ID NO: 20.

23. The epigenetic modulator of any one of claims 20-22, wherein the second repressor domain comprises any one of SEQ ID NO: 5-SEQ ID NO: 20.

24. The epigenetic modulator of any one of claims 20-22, wherein the second repressor domain comprises SEQ ID NO: 6 or SEQ ID NO: 7.

25. The epigenetic modulator of any one of claims 8-24, comprising at least 80% sequence identity to SEQ ID NO: 26 or SEQ ID NO: 34.

26. The epigenetic modulator of any one of claims 8-25, comprising at least 90% sequence identity to SEQ ID NO: 26 or SEQ ID NO: 34.

27. The epigenetic modulator of any one of claims 8-26, comprising at least 95% sequence identity to SEQ ID NO: 26 or SEQ ID NO: 34.

28. The epigenetic modulator of any one of claims 8-27, comprising SEQ ID NO: 26 or SEQ ID NO: 34.

29. The epigenetic modulator of any one of claims 8-28, comprising SEQ ID NO: 25.

30. The epigenetic modulator of any one of claims 8-28, comprising SEQ ID NO: 26.

31. The epigenetic modulator of any one of claims 8-28, comprising SEQ ID NO: 27.

32. The epigenetic modulator of any one of claims 8-28, comprising SEQ ID NO: 28.

33. A polynucleotide encoding the zinc finger protein of any one of claims 1-7.

34. A polynucleotide encoding the epigenetic modulator of any one of claims 8-32.

35. The polynucleotide of claim 33 or claim 34, further comprising a promoter.

36. The polynucleotide of claim 35, wherein the promoter comprises at least 90% sequence identity to any one of SEQ ID NO: 37-SEQ ID NO: 47, and SEQ ID NO: 98.

37. The polynucleotide of claim 35 or claim 36, wherein the promoter comprises any one of SEQ ID NO: 37-SEQ ID NO: 47 and SEQ ID NO: 98.

38. A delivery vector encapsulating the polynucleotide of any one of claims 33-37.

39. The delivery vector of claim 38, comprising a viral vector.

40. The delivery vector of claim 39, wherein the viral vector comprises a delivery-enhancing peptide.

41. The delivery vector of claim 40, wherein the delivery-enhancing peptide comprises a protoxin, a jingzhaotoxina a theraphotoxin, a phlotoxin, Grammostola porter toxin, a huwentoxin, a Ceratogyrus cornuatus toxin, a heteropodatoxin, a heteroscodratoxin, or a penetration enhancing peptide.

42. The delivery vector of claim 40 or claim 41, wherein the delivery-enhancing peptide comprises a sequence having at least 90% sequence identity to any one of SEQ ID NO: 48-SEQ ID NO: 72.

43. The delivery vector of any one of claims 39-42, wherein the viral vector comprises an AAV vector, a lentiviral vector, a Herpes Simplex Virus (HSV), or a rabies virus vector.

44. The delivery vector of claim 38, comprising a lipid nanoparticle.

45. The delivery vector of claim 44, wherein the lipid nanoparticle encapsulates DNA, mRNA, or circular RNA coding for the epigenetic modulator of any one of claims 8-32.

46. A method of downregulating a Na_{V}1.7 in a cell, the method comprising expressing in the cell the epigenetic modulator of any one of claims 8-32.

47. A method of treating a condition in a subject, the method comprising expressing in the subject the epigenetic modulator of any one of claims 8-32.

48. The method of claim 47, wherein the condition is associated with a Na_{V}1.7.

49. The method of any one of claims 47-48, wherein the condition is pain, inflammation, and/or cancer.

50. The method of any one of claims 47-49, wherein the condition is small-fiber neuropathy, back pain, rheumatoid arthritis, osteoarthritis, spinal stenosis, chronic cough, migraine, trigeminal neuralgia, erythromelalgia, and paroxysmal extreme pain disorder.

51. The method of claim 49, wherein the inflammation is associated with arthritis.

52. The method of claim 51, wherein the arthritis is rheumatoid arthritis or osteoarthritis.

53. The method of any one of claims 47-52, wherein treating inflammation comprises preventing the inflammation.

54. The method of any one of claims 47-53, wherein treating inflammation comprises reducing the inflammation.

55. The method of any one of claims 47-54, wherein treating pain comprises preventing the pain.

56. The method of any one of claims 47-55, wherein treating pain comprises reducing the pain.

57. The method of any one of claims 47-56, wherein the pain is associated with neuropathy, chemotherapy, or inflammation.

58. A method of delivering an epigenetic modulator as a purified protein to a cell, the method comprising: administering the epigenetic modulator of any one of claims 8-32 to the cell.

59. The method of claim 58, wherein the epigenetic modulator is modified to enhance cellular uptake.

60. The method of claim 59, wherein the modification comprises fusion with a cell-penetrating peptide.

61. The method of claim 60, wherein the cell-penetrating peptide comprises TAT, polyarginine, or combinations thereof.

62. A method of delivering an epigenetic modulator as RNA encapsulated in a lipid nanoparticle, exosome, or liposome, the method comprising: administering the epigenetic modulator of any one of claims 8-32 to a cell.

63. A composition for direct protein delivery, comprising:
(a) the epigenetic modulator of any one of claims 8-32; and
(b) a pharmaceutically acceptable carrier.

64. The composition of claim 63, further comprising a cell-penetrating peptide fused to the epigenetic modulator.

65. The composition of claim 63, further comprising a liposome encapsulating the epigenetic modulator.

66. A method of treating a condition in a subject, the method comprising: administering to the subject a therapeutically effective amount of the epigenetic modulator of any one of claims 8-32 via direct protein delivery.

67. The method of claim 66, wherein the epigenetic modulator is administered by a route selected from the group consisting of intravenous injection, subcutaneous injection, intramuscular injection, aerosol administration and local administration to a target tissue.

68. The method of claim 67, wherein the target tissue is selected from the group consisting of trigeminal ganglia, dorsal root ganglia, and combinations thereof.

69. The method of any one of claims 66-68, wherein the condition is selected from the group consisting of pain, inflammation, cancer, small-fiber neuropathy, back pain, rheumatoid arthritis, osteoarthritis, spinal stenosis, chronic cough, migraine, trigeminal neuralgia, erythromelalgia, and paroxysmal extreme pain disorder.

70. The method of any one of claims 66-68, wherein the condition is trigeminal neuralgia.


SEQUENCE LISTING (181 sequences)

SEQ ID NO: 1 - protein, 178 aa, synthetic construct
MAERPFQCRICMRNFSRSDVLSRHIRTHTGEKPFACDICGRKFADSRDRKNHTKIHTGSQKPFQCRICMRNFSRSADLTRHIRTHTGEKPFACDICGRKFADRSHLARHTKIHTGSQKPFQCRICMRNFSRSDNLSEHIRTHTGEKPFACDICGRKFASKQYLIKHTKIHLRQKDAAR

SEQ ID NO: 2 - protein, 236 aa, synthetic construct
LEPGEKPYKCPECGKSFSTSGNLVRHQRTHTGEKPYKCPECGKSFSTSGNLVRHQRTHMAERPFQCRICMRNFSRSDKLVRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAAR

SEQ ID NO: 3 - protein, 178 aa, synthetic construct
MAERPFQCRICMHNFSRSDVLSRHIRTHTGEKPFACDICGHKFADSRDRKNHTKIHTGSQKPFQCRICMHNFSRSADLTRHIRTHTGEKPFACDICGHKFADRSHLARHTKIHTGSQKPFQCRICMHNFSRSDNLSEHIRTHTGEKPFACDICGHKFASKQYLIKHTKIHLRQKDAAR

SEQ ID NO: 4 - protein, 235 aa, synthetic construct
LEPGEKPYKCPECGKSFSTSGNLVRHQRTHTGEKPYKCPECGKSFSTSGNLVRHQRTMAERPFQCRICMQNFSRSDKLVRHIRTHTGEKPFACDICGQKFATSGHLSRHTKIHTGSQKPFQCRICMQNFSRSDALSEHIRTHTGEKPFACDICGQKFAQNATRTKHTKIHTGSQKPFQCRICMQNFSTSGHLSRHIRTHTGEKPFACDICGQKFAQSGDLTRHTKIHLRQKDAAR

SEQ ID NO: 5 - protein, 65 aa, Homo sapiens
RTLVTFKDVFVDFTREEWKLLDTAQQIVYRNVMLENYKNLVSLGYQLTKPDVILRLEKGEEPWLV

SEQ ID NO: 6 - protein, 29 aa, Homo sapiens
MNIQMLLEAADYLERREREAEHGYASMLP

SEQ ID NO: 7 - protein, 66 aa, synthetic construct
MNIQMLLEAADYLERREREAEHGYASMLPGGGSGGGSMNIQMLLEAADYLERREREAEHGYASMLP

SEQ ID NO: 8 - protein, 62 aa, Homo sapiens
VTFEDVTVNFTQGEWQRLNPEQRNLYRDVMLENYSNLVSVGQGETTKPDVILRLEQGKEPWL

SEQ ID NO: 9 - protein, 62 aa, Homo sapiens
VTFKDVFVDFTREEWKLLDTAQQIVYRNVMLENYKNLVSLGYQLTKPDVILRLEKGEEPWLV

SEQ ID NO: 10 - protein, 62 aa, Homo sapiens
VTFEDVSMDFSQEEWELLEPAQKNLYREVMLENYRNVVSLEALKNQCTDVGIKEGPLSPAQT

SEQ ID NO: 11 - protein, 61 aa, Homo sapiens
VTFDDVAVTFTKEEWGQLDLAQRTLYQEVMLENCGLLVSLGCPVPKAELICHLEHGQEPWT

SEQ ID NO: 12 - protein, 61 aa, Homo sapiens
MAFEDVAVYFSQEEWGLLDTAQRALYRRVMLDNFALVASLGLSTSRPRVVIQLERGEEPWV

SEQ ID NO: 13 - protein, 286 aa, Homo sapiens
VQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 14 - protein, 220 aa, Homo sapiens
ARYLGNTVDLSSFDFRTGKMMPSKLQKNKQRLRNDPLNQNKGKPDLNTTLPIRQTASIFKQPVTKVTNHPSNKVKSDPQRMNEQPRQLFWEKRLQGLSASDVTEQIIKTMELPKGLQGVGPGSNDETLLSAVASALHTSSAPITGQVSAAVEKNPAVWLNTSQPLCKAFIVTDEDIRKQEERVQQVRKILEDALMADILSRAADTEEMDIEMDSGDEAEF

SEQ ID NO: 15 - protein, 29 aa, Homo sapiens
MNIQMLLEAADYLERREREAEHGYASMLP

SEQ ID NO: 16 - protein, 121 aa, Homo sapiens
MKEGENNKPREKSEGNKRKSSFSNSADDIKSKKKREQSNDIARGFERGLEPEKIIGATDSCGDLMFLMKWKDTDEADLVLAKEANVKCPQIVIAFYEERLTWHAYPEDAENKEKESAKSEF

SEQ ID NO: 17 - protein, 267 aa, Homo sapiens
SSSMADFRKFFAKAKHIVIISGAGVSAESGVPTFRGAGGYWRKWQAQDLATPLAFAHNPSRVWEFYHYRREVGSKEPNAGHRAIAECETRLGKQGRRVWITQNIDELHRKAGTKNLLEIHGSLFKTRCTSCGWAENYKSPICPALSGKGAPEPGTQDASIPVEKLPRCEEAGCGGLLRPHWWFGENLDPAILEEVDRELAHCDLCLWGTSSWYPAAFAPQVAARGVPVAEFNTETTPATNRFRFHFQGPCGTTLPEALACHENETVS

SEQ ID NO: 18 - protein, 209 aa, Homo sapiens
SCDSTNAAIAKQALKKPIKGKQAPRKKAQGKTQQNRKLTDFYPVRRSSRKSKAELQSEERKRIDELIESGKEEGKIDLIDGKGRGVIATKQFSRGDFVVEYHGDLIEITDAKKREALYAQDPSTGCYYYFQYLSKTYCVDATRETNRLGRLINHSKCGNCQTKLHDIDGVPHLILIASRDIAAGEELLYDYGDRSKASIEAFPWLKHEF

SEQ ID NO: 19 - protein, 213 aa, Homo sapiens
MEFWGIEVKSGKPVTVTPEEGILIHVSQASLGECKNKKGEFVPLHVKVGNQNLVLGTLSTENIPQLFCDLVFDKEFELSHTWGKGSVYFVGYKTPNIEPQGYSEEEEEEEEEVPAGNAAKAVAKPKAKPAEVKPAVDDEEDESDSDGMDEDDSDGEDSEEEEPTPKKPASSKKRANETTPKAPVSAKKAKVAVTPQKTDEKKKGGKAANQSEF

SEQ ID NO: 20 - protein, 6 aa, Homo sapiens
DLELRL

SEQ ID NO: 21 - protein, 7 aa, Simian virus 40
PKKKRKV

SEQ ID NO: 22 - protein, 27 aa, synthetic construct
PKKKRKVLEPKKKRKVPGMAPKKKRKV

SEQ ID NO: 23 - protein, 8 aa, synthetic construct
DYKDDDDK

SEQ ID NO: 24 - protein, 23 aa, synthetic construct
MDYKDHDGDYKDHDIDYKDDDDK

SEQ ID NO: 25 - protein, 278 aa, synthetic construct
MGSPKKKRKVLEPKKKRKVPGMAPKKKRKVGSSMAERPFQCRICMRNFSRSDVLSRHIRTHTGEKPFACDICGRKFADSRDRKNHTKIHTGSQKPFQCRICMRNFSRSADLTRHIRTHTGEKPFACDICGRKFADRSHLARHTKIHTGSQKPFQCRICMRNFSRSDNLSEHIRTHTGEKPFACDICGRKFASKQYLIKHTKIHLRQKDAARGSRTLVTFKDVFVDFTREEWKLLDTAQQIVYRNVMLENYKNLVSLGYQLTKPDVILRLEKGEEPWLV

SEQ ID NO: 26 - protein, 336 aa, synthetic construct
MGSPKKKRKVLEPKKKRKVPGMAPKKKRKVGSSLEPGEKPYKCPECGKSFSTSGNLVRHQRTHTGEKPYKCPECGKSFSTSGNLVRHQRTHMAERPFQCRICMRNFSRSDKLVRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAARGSRTLVTFKDVFVDFTREEWKLLDTAQQIVYRNVMLENYKNLVSLGYQLTKPDVILRLEKGEEPWLV

SEQ ID NO: 27 - protein, 278 aa, synthetic construct
MGSPKKKRKVLEPKKKRKVPGMAPKKKRKVGSSMAERPFQCRICMHNFSRSDVLSRHIRTHTGEKPFACDICGHKFADSRDRKNHTKIHTGSQKPFQCRICMHNFSRSADLTRHIRTHTGEKPFACDICGHKFADRSHLARHTKIHTGSQKPFQCRICMHNFSRSDNLSEHIRTHTGEKPFACDICGHKFASKQYLIKHTKIHLRQKDAARGSRTLVTFKDVFVDFTREEWKLLDTAQQIVYRNVMLENYKNLVSLGYQLTKPDVILRLEKGEEPWLV

SEQ ID NO: 28 - protein, 335 aa, synthetic construct
MGSPKKKRKVLEPKKKRKVPGMAPKKKRKVGSSLEPGEKPYKCPECGKSFSTSGNLVRHQRTHTGEKPYKCPECGKSFSTSGNLVRHQRTMAERPFQCRICMQNFSRSDKLVRHIRTHTGEKPFACDICGQKFATSGHLSRHTKIHTGSQKPFQCRICMQNFSRSDALSEHIRTHTGEKPFACDICGQKFAQNATRTKHTKIHTGSQKPFQCRICMQNFSTSGHLSRHIRTHTGEKPFACDICGQKFAQSGDLTRHTKIHLRQKDAARGSRTLVTFKDVFVDFTREEWKLLDTAQQIVYRNVMLENYKNLVSLGYQLTKPDVILRLEKGEEPWLV

SEQ ID NO: 29 - protein, 295 aa, synthetic construct
MDYKDHDGDYKDHDIDYKDDDDKMAPKKKRKVGIHGVPAAMAERPFQCRICMRNFSRSDVLSRHIRTHTGEKPFACDICGRKFADSRDRKNHTKIHTGSQKPFQCRICMRNFSRSADLTRHIRTHTGEKPFACDICGRKFADRSHLARHTKIHTGSQKPFQCRICMRNFSRSDNLSEHIRTHTGEKPFACDICGRKFASKQYLIKHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 30 - protein, 353 aa, synthetic construct
MDYKDHDGDYKDHDIDYKDDDDKMAPKKKRKVGIHGVPAALEPGEKPYKCPECGKSFSTSGNLVRHQRTHTGEKPYKCPECGKSFSTSGNLVRHQRTHMAERPFQCRICMRNFSRSDKLVRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 31 - protein, 295 aa, synthetic construct
MDYKDHDGDYKDHDIDYKDDDDKMAPKKKRKVGIHGVPAAMAERPFQCRICMHNFSRSDVLSRHIRTHTGEKPFACDICGHKFADSRDRKNHTKIHTGSQKPFQCRICMHNFSRSADLTRHIRTHTGEKPFACDICGHKFADRSHLARHTKIHTGSQKPFQCRICMHNFSRSDNLSEHIRTHTGEKPFACDICGHKFASKQYLIKHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 32 - protein, 352 aa, synthetic construct
MDYKDHDGDYKDHDIDYKDDDDKMAPKKKRKVGIHGVPAALEPGEKPYKCPECGKSFSTSGNLVRHQRTHTGEKPYKCPECGKSFSTSGNLVRHQRTMAERPFQCRICMQNFSRSDKLVRHIRTHTGEKPFACDICGQKFATSGHLSRHTKIHTGSQKPFQCRICMQNFSRSDALSEHIRTHTGEKPFACDICGQKFAQNATRTKHTKIHTGSQKPFQCRICMQNFSTSGHLSRHIRTHTGEKPFACDICGQKFAQSGDLTRHTKIHLRQKDAARGSRTLVTFKDVFVDFTREEWKLLDTAQQIVYRNVMLENYKNLVSLGYQLTKPDVILRLEKGEEPWLVDYKDDDDKRS

SEQ ID NO: 33 - protein, 354 aa, synthetic construct
MGSPKKKRKVLEPKKKRKVPGMAPKKKRKVGSSRTLVTFKDVFVDFTREEWKLLDTAQQIVYRNVMLENYKNLVSLGYQLTKPDVILRLEKGEEPWLVGGGSGTMAERPFQCRICMRNFSRSDVLSRHIRTHTGEKPFACDICGRKFADSRDRKNHTKIHTGSQKPFQCRICMRNFSRSADLTRHIRTHTGEKPFACDICGRKFADRSHLARHTKIHTGSQKPFQCRICMRNFSRSDNLSEHIRTHTGEKPFACDICGRKFASKQYLIKHTKIHLRQKDAARGTGGGSMNIQMLLEAADYLERREREAEHGYASMLPGGGSGGGSMNIQMLLEAADYLERREREAEHGYASMLP

SEQ ID NO: 34 - protein, 412 aa, synthetic construct
MGSPKKKRKVLEPKKKRKVPGMAPKKKRKVGSSRTLVTFKDVFVDFTREEWKLLDTAQQIVYRNVMLENYKNLVSLGYQLTKPDVILRLEKGEEPWLVGGGSGTLEPGEKPYKCPECGKSFSTSGNLVRHQRTHTGEKPYKCPECGKSFSTSGNLVRHQRTHMAERPFQCRICMRNFSRSDKLVRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAARGTGGGSMNIQMLLEAADYLERREREAEHGYASMLPGGGSGGGSMNIQMLLEAADYLERREREAEHGYASMLP

SEQ ID NO: 35 - protein, 354 aa, synthetic construct
MGSPKKKRKVLEPKKKRKVPGMAPKKKRKVGSSRTLVTFKDVFVDFTREEWKLLDTAQQIVYRNVMLENYKNLVSLGYQLTKPDVILRLEKGEEPWLVGGGSGTMAERPFQCRICMHNFSRSDVLSRHIRTHTGEKPFACDICGHKFADSRDRKNHTKIHTGSQKPFQCRICMHNFSRSADLTRHIRTHTGEKPFACDICGHKFADRSHLARHTKIHTGSQKPFQCRICMHNFSRSDNLSEHIRTHTGEKPFACDICGHKFASKQYLIKHTKIHLRQKDAARGTGGGSMNIQMLLEAADYLERREREAEHGYASMLPGGGSGGGSMNIQMLLEAADYLERREREAEHGYASMLP

SEQ ID NO: 36 - protein, 411 aa, synthetic construct
MGSPKKKRKVLEPKKKRKVPGMAPKKKRKVGSSRTLVTFKDVFVDFTREEWKLLDTAQQIVYRNVMLENYKNLVSLGYQLTKPDVILRLEKGEEPWLVGGGSGTLEPGEKPYKCPECGKSFSTSGNLVRHQRTHTGEKPYKCPECGKSFSTSGNLVRHQRTMAERPFQCRICMQNFSRSDKLVRHIRTHTGEKPFACDICGQKFATSGHLSRHTKIHTGSQKPFQCRICMQNFSRSDALSEHIRTHTGEKPFACDICGQKFAQNATRTKHTKIHTGSQKPFQCRICMQNFSTSGHLSRHIRTHTGEKPFACDICGQKFAQSGDLTRHTKIHLRQKDAARGTGGGSMNIQMLLEAADYLERREREAEHGYASMLPGGGSGGGSMNIQMLLEAADYLERREREAEHGYASMLP

SEQ ID NO: 37 - DNA, 600 bp, synthetic construct
  source 1..600: mol_type = other DNA
cagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgc

SEQ ID NO: 38 - DNA, 724 bp, synthetic construct
  source 1..724: mol_type = other DNA
cttgagcgtcaccgccccactcgcggttgtgagcaaagccctacggaagaaaccaattcccagcctagactcttcagagcccaaggtcggggaggcgctggcctggcggtgttgtctggctccccagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgc

SEQ ID NO: 39 - DNA, 524 bp, synthetic construct
  source 1..524: mol_type = other DNA
gagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagagagcgccggggaaggagaggcccgcgccctctcctggagccagattctgcaggtgcactgggtggggatgatcggcgggctaggttgcaagtaagtgccttttcttttgctgctttctttatttctttgtttccatccaggcctcttatgtgaggagctgaagaggaattaaaatatacaggatgaaaaggcggccgccatggtgagcaagggcgaggaggataa

SEQ ID NO: 40 - DNA, 1000 bp, synthetic construct
  source 1..1000: mol_type = other DNA
tcgaattaacacaagacagaagtcatataaatcttaagaaggtagaaaatgttacttctagaaaactccaccaaaagttggttggataatttcaggtcttctttctggaaaacgaagagtcacaaagtctatttttctttctactcccaagttccactcattctccaattaaatgtgactcataggtgactgaatgtcatgttttggccactctaagcaaagggattccttggacttgggtacacatattttaagaatattgtatatataagaatattgtatatattcaactgtccatcttgctgcttctctcttacaccaacttcattttgtagcatcagctgcataagctcttgcttttccccctatgcagacatctattcatcaggctctgtgtacactggcaacataggaataaactggataaggttgctgttcccagaagaatctcagaatattgtatttatttaatggcatcaggaaaaaccagtccaccaattttagaaagaaaaaagtcttaaagctgcacatttttatgaacttagacaattactatattttgactattattgactctaatctattccaagaattatacagacagaaagagctttggtaacagcctacatatcaatatttactgaaccaagaactatcacaaaacgtctgttgaaaatttccatgttttaatgatgatgatgctaatggcagctatgatttattaaacactttctatgtgcaggatgtaagctaagtaatttgtacacattatcttatttaaacttattacaagtcttattttcttgaggtagatgttattatcctattttcacagacagacttatgccttgaagaatttggtaaagtcactaagtagtcagtagccaaatcatgtattcttaatccttattcaatattgtccccctatagaagaaaccttgagtttgtaacttttcagtaaggctatttagcttgtgtcctgaagacactctcacctata

SEQ ID NO: 41 - DNA, 1000 bp, synthetic construct; identical to SEQ ID NO: 40
  source 1..1000: mol_type = other DNA

SEQ ID NO: 42 - DNA, 1000 bp, synthetic construct
  source 1..1000: mol_type = other DNA
tcaattataacagacatgtcttagtctaccttttattgctgtaactgaatacctgagactggttaaagtattcaataaagaaatttatttcttaaagttctggaagctgggaagtctaaggtgaagggtccacatctggtgggaactttcttgctggtgggcacaccagagagtcccaaggcagctcatggcatcacatggcgaggggtgttcactgagagccaaactgggttttataataaacccactcttgtgatatctaactcactcccatgataacccattaagcccttactctatattccattcatgaaggcagaaccttcatgacctcatcaccttttaaaggctctgcctcttaatactgttacattgggaattaagttccaacatgagtttcaaaagggacaaacattcaaaccacagcaaaacattaaaaaaaaaattttctgattaaagagggagcatacatattttcaagtgttcttaagtggaaaataatggatgtgaatgactgggcaaatttaagagtggcatgaggagtaatttattctgctaaatacgtagctttaggaatttaaaaaaaaaagccatgttacatttagtgagacatctggaaatactacatgaggatgagcactttaaaacacttcccgcttcatcagtcagtttacctgtctctgatggtaacagcaatgaactttttgtgaacaatgactttttctacgcctgaaatagataaatatataaagttggctctcatctgttcctattcttccctcgaaaaattgaatctctcagtcactaacttattcgtaatgaagaactgcattccagacatttttcttagacatcgtctcctgctacacaggatgtgaggcaaatgaatattttccatggaactaattcagagagtaaacatatttatagatccagatctaaggtcatggaggagctcattgctttgtaactaaaatgtgtattgattgtattgctgttc

SEQ ID NO: 43 - DNA, 1440 bp, synthetic construct
  source 1..1440: mol_type = other DNA
cagtctcagtcactgaagtttccttccttcaatagcacatcttacaagtcaagaatgtaaggtccaaagccccaggacaaatctcagaagcaaagaaagcagcagggaaaagtagaccctgggatttgatttccttccggttatctctaagcatcatttccatgatagaaggtgtggaagcaaataataaaagtgcccgtcactagtgtttatcctgcaaagtggtctgcccttttgagaggcaccctgccctatggccatcctttgatttcttcccttggtggaaatttcctgtttctctttgagaaagataattcaaccctgtttccattgttcttcctcccaggctgctgtaggaaagcgcacgcacacactccacacaaaatgaatttttaaaaaatttattttcacagtcgctcctaccagctctgaaattcagaacccatatgactgatggcatattcagataatcgggtcccaggtctggaaaagcagccttttccccacgtttctttccccacctaggacctcctctgattcttcactgcatcttcgaaagaaaatgtattatttgcttgcctggaagacgctgcaattcaattgattttatatatacatatatataaagaaaacagaaaacatagcctagataccggtcttgagcgtcaccgccccactcgcggttgtgagcaaagccctacggaagaaaccaattcccagcctagactcttcagagcccaaggtcggggaggcgctggcctggcggtgttgtctggctccccagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacctccacgggcgcgccctggcaggaggggcgcagtctgcttgcaggcggtcgccagcgctccagcggcggctgtcggctttccaattccgccagctcggctgaggctgggctagcctgggtgccagtggctgctagcggcaggcgtcccctgagcaacaggagcccagagaaaaagaagcagccctgagaga

SEQ ID NO: 44 - DNA, 449 bp, synthetic construct
  source 1..449: mol_type = other DNA
cagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 45 - DNA, 607 bp, synthetic construct
  source 1..607: mol_type = other DNA
ctcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaact

SEQ ID NO: 46 - DNA, 164 bp, synthetic construct
  source 1..164: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 47 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 48 - protein, 30 aa, synthetic construct
YCQKWMWTCDSERKCCEGMVCRLWCKKKLW

SEQ ID NO: 49 - protein, 32 aa, synthetic construct
GPYCQKWMQTCDSERKCCEGMVCRLWCKKKLL

SEQ ID NO: 50 - protein, 35 aa, synthetic construct
DCRYMFGDCEKDEDCCKHLGCKRKMKYCAWDFTFT

SEQ ID NO: 51 - protein, 29 aa, synthetic construct
YCQKWMWTCDSKRACCEGLRCKLWCRKII

SEQ ID NO: 52 - protein, 29 aa, synthetic construct
YCQKWMWTCDSKRACCEGLRCKLWCRKEI

SEQ ID NO: 53 - protein, 29 aa, synthetic construct
YCQKWLWTCDSERKCCEDMVCRLWCKKRL

SEQ ID NO: 54 - protein, 34 aa, synthetic construct
ACLGQWDSCDPKASKCCPNYACEWKYPWCRYKLF

SEQ ID NO: 55 - protein, 34 aa, synthetic construct
ACLGQWASCDPKASKCCPNYACEWKYPWCRYKLF

SEQ ID NO: 56 - protein, 33 aa, synthetic construct
DCLKFGWKCNPRNDKCCSGLKCGSNHNWCKLHL

SEQ ID NO: 57 - protein, 34 aa, synthetic construct
DCLGFMRKCIPDNDKCCRPNLVCSRTHKWCKYVF

SEQ ID NO: 58 - protein, 34 aa, synthetic construct
DCLGFFRKCIPDNDKCCRPNLVCSRLHRWCKYVF

SEQ ID NO: 59 - protein, 34 aa, synthetic construct
DCLGAMRKCIPDNDKCCRPNLVCSRTHKWCKYVF

SEQ ID NO: 60 - protein, 35 aa, synthetic construct
ECLEIFKACNPSNDQCCKSSKLVCSRKTRWCKYQI

SEQ ID NO: 61 - protein, 35 aa, synthetic construct
GCLGIFKACNPSNDQCCKSSKLVCSRKTRWCKWQI

SEQ ID NO: 62 - protein, 33 aa, synthetic construct
DCLGMFKSCDPENDKCCKRLVCSRSHRWCKWKL

SEQ ID NO: 63 - protein, 33 aa, synthetic construct
ICLGMFKSCDPENDKCCKRLVCSRSHRWCKWKL

SEQ ID NO: 64 - protein, 33 aa, synthetic construct
DCGTIWHYCGTDQSECCEGWKCSRQLCKYVIDW

SEQ ID NO: 65 - protein, 35 aa, synthetic construct
ECRYLFGGCSSTSDCCKHLSCRSDWKYCAWDGTFS

SEQ ID NO: 66 - protein, 29 aa, synthetic construct
  SITE 6: note = Norleucine
  SITE 17: note = Propargylglycine
YCQKWXWTCDSKRACCXGLRCKLWCRKEI

SEQ ID NO: 67 - protein, 65 aa, synthetic construct
GVRDAYIADDKNCVYTCASNGYCNTECTKNGAESGYCQWIGRYGNACWCIKLPDEVPIRIPGKCR

SEQ ID NO: 68 - protein, 18 aa, synthetic construct
LLIILRRRIRKQAHAHSK

SEQ ID NO: 69 - protein, 21 aa, synthetic construct
KETWWETWWTEWSQPKKKRKV

SEQ ID NO: 70 - protein, 18 aa, synthetic construct
KLALKLALKALKAALKLA

SEQ ID NO: 71 - protein, 28 aa, synthetic construct
LSTAADMQGVVTDGMASGLDKDYLKPDD

SEQ ID NO: 72 - protein, 32 aa, synthetic construct
LLKKKCWLRCVMGECCKRESDCTQMWKQCYPG

SEQ ID NO: 73 - DNA, 18 bp, Homo sapiens
  source 1..18: mol_type = genomic DNA
agtctgcttgcaggcggt

SEQ ID NO: 74 - DNA, 18 bp, Homo sapiens
  source 1..18: mol_type = genomic DNA
ccagcggcggctgtcggc

SEQ ID NO: 75 - DNA, 18 bp, Homo sapiens
  source 1..18: mol_type = genomic DNA
gcctgggtgccagtggct

SEQ ID NO: 76 - DNA, 18 bp, Homo sapiens
  source 1..18: mol_type = genomic DNA
tggctgctagcggcaggc

SEQ ID NO: 77 - DNA, 18 bp, Homo sapiens
  source 1..18: mol_type = genomic DNA
aaggagaggcccgcgccc

SEQ ID NO: 78 - DNA, 18 bp, Homo sapiens
  source 1..18: mol_type = genomic DNA
gcaggtgcactgggtggg

SEQ ID NO: 79 - DNA, 18 bp, Homo sapiens
  source 1..18: mol_type = genomic DNA
tgccagggcgcgcccgtg

SEQ ID NO: 80 - DNA, 18 bp, Homo sapiens
  source 1..18: mol_type = genomic DNA
acagccgccgctggagcg

SEQ ID NO: 81 - DNA, 18 bp, Homo sapiens
  source 1..18: mol_type = genomic DNA
ccaggagagggcgcgggc

SEQ ID NO: 82 - DNA, 18 bp, Mus musculus
  source 1..18: mol_type = genomic DNA
ggcgaggtgatggaaggg

SEQ ID NO: 83 - DNA, 18 bp, Mus musculus
  source 1..18: mol_type = genomic DNA
gagggagctaggggtggg

SEQ ID NO: 84 - DNA, 18 bp, Mus musculus
  source 1..18: mol_type = genomic DNA
agtgctaatgtttccgag

SEQ ID NO: 85 - DNA, 18 bp, Mus musculus
  source 1..18: mol_type = genomic DNA
tagacggtgcagggcgga

SEQ ID NO: 86 - DNA, 18 bp, Homo sapiens
  source 1..18: mol_type = genomic DNA
gtgggtgtggcggttgag

SEQ ID NO: 87 - DNA, 18 bp, Homo sapiens
  source 1..18: mol_type = genomic DNA
gccgccgctggagcgctg

SEQ ID NO: 88 - DNA, 18 bp, Homo sapiens
  source 1..18: mol_type = genomic DNA
gcgcccgtggaggtagca

SEQ ID NO: 89 - DNA, 18 bp, Homo sapiens
  source 1..18: mol_type = genomic DNA
gccgctggagcgctggcg

SEQ ID NO: 90 - DNA, 18 bp, Homo sapiens
  source 1..18: mol_type = genomic DNA
ggtgtggcggttgaggcg

SEQ ID NO: 91 - DNA, 18 bp, Homo sapiens
  source 1..18: mol_type = genomic DNA
ggggaaggagaggcccgc

SEQ ID NO: 92 - DNA, 18 bp, Homo sapiens
  source 1..18: mol_type = genomic DNA
gcgtcccctgagcaacag

SEQ ID NO: 93 - DNA, 18 bp, Homo sapiens
  source 1..18: mol_type = genomic DNA
tcagccgagctggcggaa

SEQ ID NO: 94 - DNA, 18 bp, Homo sapiens
  source 1..18: mol_type = genomic DNA
gctggagcgctggcgacc

SEQ ID NO: 95 - DNA, 18 bp, Homo sapiens
  source 1..18: mol_type = genomic DNA
ggtggggatgatcggcgg

SEQ ID NO: 96 - DNA, 18 bp, Homo sapiens
  source 1..18: mol_type = genomic DNA
tggttccagcaatgggag

SEQ ID NO: 97 - DNA, 18 bp, Homo sapiens
  source 1..18: mol_type = genomic DNA
ggagagggcgcgggcctc

SEQ ID NO: 98 - DNA, 469 bp, synthetic construct
  source 1..469: mol_type = other DNA
cagccactgccccagactcagggctttgccattggtccccacctcctctgctccggagtttttctccagctccccaccaagccacacaaagtgacttctcggaaacattagccgattctgctgagcaggaagggaggaaagggatgatgggggcgggggtgagataagggaagggctcttctggctgctggacacacacacacacacactcaaacacacacacgccccacccaatgggtggccgtggatggcaggtcgtgcaaccccctcctccgccttctattagcgcatggtgcagaggctacagcgtcgccaccaccgcgcccctagctgggtccccgccctgcgccgcccgcaggagtggagagagggagggagggagggagcaaggggtggggacccgggcgcgctgggaggagtggaggaggcaaagcggcgcagctgccctcggggaggcggggctgctacc

SEQ ID NO: 99 - protein, 178 aa, synthetic construct
MAERPFQCRICMRNFSTSGHLSRHIRTHTGEKPFACDICGRKFADRSHLARHTKIHTGSQKPFQCRICMRNFSQSGDLTRHIRTHTGEKPFACDICGRKFAYRWLRNNHTKIHTGSQKPFQCRICMRNFSRSDTLSEHIRTHTGEKPFACDICGRKFARAQHLQQHTKIHLRQKDAAR

SEQ ID NO: 100 - protein, 178 aa, synthetic construct
MAERPFQCRICMRNFSDRSHLSRHIRTHTGEKPFACDICGRKFADRSALARHTKIHTGSQKPFQCRICMRNFSQSSDLSRHIRTHTGEKPFACDICGRKFARSDDLTRHTKIHTGSQKPFQCRICMRNFSRSDDLTRHIRTHTGEKPFACDICGRKFAQRSTLSSHTKIHLRQKDAAR

SEQ ID NO: 101 - protein, 178 aa, synthetic construct
MAERPFQCRICMRNFSQSSDLSRHIRTHTGEKPFACDICGRKFARSDALARHTKIHTGSQKPFQCRICMRNFSQSGDLTRHIRTHTGEKPFACDICGRKFARSWGLQVHTKIHTGSQKPFQCRICMRNFSRSDHLSQHIRTHTGEKPFACDICGRKFADSSTRKKHTKIHLRQKDAAR

SEQ ID NO: 102 - protein, 178 aa, synthetic construct
MAERPFQCRICMRNFSDRSHLTRHIRTHTGEKPFACDICGRKFAQSGDLTRHTKIHTGSQKPFQCRICMRNFSRSDDLTRHIRTHTGEKPFACDICGRKFAQRSTLSSHTKIHTGSQKPFQCRICMRNFSRSDVLSEHIRTHTGEKPFACDICGRKFARNQHRKTHTKIHLRQKDAAR

SEQ ID NO: 103 - protein, 178 aa, synthetic construct
MAERPFQCRICMRNFSRSDNLSEHIRTHTGEKPFACDICGRKFAERANRNSHTKIHTGSQKPFQCRICMRNFSRSDNLARHIRTHTGEKPFACDICGRKFAWRGDRVKHTKIHTGSQKPFQCRICMRNFSDRSDLSRHIRTHTGEKPFACDICGRKFARRTDLRRHTKIHLRQKDAAR

SEQ ID NO: 104 - protein, 178 aa, synthetic construct
MAERPFQCRICMRNFSDRSDLSRHIRTHTGEKPFACDICGRKFARRTDLRRHTKIHTGSQKPFQCRICMRNFSDRSDLSRHIRTHTGEKPFACDICGRKFARSHHLKAHTKIHTGSQKPFQCRICMRNFSRSANLARHIRTHTGEKPFACDICGRKFARSDNLREHTKIHLRQKDAAR

SEQ ID NO: 105 - protein, 178 aa, synthetic construct
MAERPFQCRICMRNFSRSAHLSRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAAR

SEQ ID NO: 106 - protein, 178 aa, synthetic construct
MAERPFQCRICMRNFSQSGDLTRHIRTHTGEKPFACDICGRKFAQSGARNIHTKIHTGSQKPFQCRICMRNFSRSANLARHIRTHTGEKPFACDICGRKFARSDALARHTKIHTGSQKPFQCRICMRNFSDRSDLSRHIRTHTGEKPFACDICGRKFARRTDLRRHTKIHLRQKDAAR

SEQ ID NO: 107 - protein, 178 aa, synthetic construct
MAERPFQCRICMRNFSRSADLTRHIRTHTGEKPFACDICGRKFAQSGHLSRHTKIHTGSQKPFQCRICMRNFSQSSDLSRHIRTHTGEKPFACDICGRKFAHRKSLSRHTKIHTGSQKPFQCRICMRNFSDRSDLSRHIRTHTGEKPFACDICGRKFAQSSTRARHTKIHLRQKDAAR

SEQ ID NO: 108 - protein, 178 aa, synthetic construct
MAERPFQCRICMRNFSDRSHLTRHIRTHTGEKPFACDICGRKFARSDDLTRHTKIHTGSQKPFQCRICMRNFSDRSHLTRHIRTHTGEKPFACDICGRKFARSDNLTRHTKIHTGSQKPFQCRICMRNFSQSGHLARHIRTHTGEKPFACDICGRKFAQKGTLGEHTKIHLRQKDAAR

SEQ ID NO: 109 - protein, 178 aa, synthetic construct
MAERPFQCRICMRNFSRSAHLSRHIRTHTGEKPFACDICGRKFAQSGNLARHTKIHTGSQKPFQCRICMRNFSRSDAMSQHIRTHTGEKPFACDICGRKFARNASRTRHTKIHTGSQKPFQCRICMRNFSRSANLARHIRTHTGEKPFACDICGRKFADRSHLARHTKIHLRQKDAAR

SEQ ID NO: 110 - protein, 178 aa, synthetic construct
MAERPFQCRICMRNFSRSANLARHIRTHTGEKPFACDICGRKFADSSDRKKHTKIHTGSQKPFQCRICMRNFSTSGSLSRHIRTHTGEKPFACDICGRKFAHSLSLKNHTKIHTGSQKPFQCRICMRNFSQSSDLSRHIRTHTGEKPFACDICGRKFAWKWNLRAHTKIHLRQKDAAR

SEQ ID NO: 111 - protein, 178 aa, synthetic construct
MAERPFQCRICMRNFSRSAHLSRHIRTHTGEKPFACDICGRKFATSGHLSRHTKIHTGSQKPFQCRICMRNFSRSDHLSQHIRTHTGEKPFACDICGRKFAASSTRTKHTKIHTGSQKPFQCRICMRNFSQSSHLTRHIRTHTGEKPFACDICGRKFARSDNLTRHTKIHLRQKDAAR

SEQ ID NO: 112 - protein, 178 aa, synthetic construct
MAERPFQCRICMRNFSDRSHLTRHIRTHTGEKPFACDICGRKFADRSHLARHTKIHTGSQKPFQCRICMRNFSRSDNLSEHIRTHTGEKPFACDICGRKFARSAALARHTKIHTGSQKPFQCRICMRNFSRSDTLSQHIRTHTGEKPFACDICGRKFATRDHRIKHTKIHLRQKDAAR

SEQ ID NO: 113 - protein, 178 aa, synthetic construct
MAERPFQCRICMRNFSRSDNLARHIRTHTGEKPFACDICGRKFATSGSLTRHTKIHTGSQKPFQCRICMRNFSRSDDLSKHIRTHTGEKPFACDICGRKFARSDALARHTKIHTGSQKPFQCRICMRNFSTSGHLSRHIRTHTGEKPFACDICGRKFARSDALARHTKIHLRQKDAAR

SEQ ID NO: 114 - protein, 178 aa, synthetic construct
MAERPFQCRICMRNFSRSDALSEHIRTHTGEKPFACDICGRKFARSADRTRHTKIHTGSQKPFQCRICMRNFSQSGHLARHIRTHTGEKPFACDICGRKFAHRSTLSRHTKIHTGSQKPFQCRICMRNFSDRSDLSRHIRTHTGEKPFACDICGRKFADRSDLSRHTKIHLRQKDAAR

SEQ ID NO: 115 - protein, 180 aa, synthetic construct
MAERPFQCRICMRNFSQSGDLTRHIRTHTGEKPFACDICGRKFAQSGARNIHTKIHTGSQKPFQCRICMRNFSRSANLARHIRTHTGEKPFACDICGRKFARSDALARHTKIHTGSQKPFQCRICMRNFSDRSDLSRHIRTHTGEKPFACDICGRKFARRTDLRRHTKIHLRQKDAARGS

SEQ ID NO: 116 - protein, 178 aa, synthetic construct
MAERPFQCRICMRNFSRSDNLSRHIRTHTGEKPFACDICGRKFAHSATRKRHTKIHTGSQKPFQCRICMRNFSRSDDLSKHIRTHTGEKPFACDICGRKFARSDALARHTKIHTGSQKPFQCRICMRNFSRSSHLARHIRTHTGEKPFACDICGRKFARSDALARHTKIHLRQKDAAR

SEQ ID NO: 117 - protein, 178 aa, synthetic construct
MAERPFQCRICMRNFSRSDTLTRHIRTHTGEKPFACDICGRKFARSDARTNHTKIHTGSQKPFQCRICMRNFSRSSDLTRHIRTHTGEKPFACDICGRKFAQSGHLSRHTKIHTGSQKPFQCRICMRNFSRSDTLTQHIRTHTGEKPFACDICGRKFADSSDLSRHTKIHLRQKDAAR

SEQ ID NO: 118 - protein, 178 aa, synthetic construct
MAERPFQCRICMRNFSRSDDLTRHIRTHTGEKPFACDICGRKFARSDNLARHTKIHTGSQKPFQCRICMRNFSQSSALSRHIRTHTGEKPFACDICGRKFARSDHLSRHTKIHTGSQKPFQCRICMRNFSRSDSLARHIRTHTGEKPFACDICGRKFANRHHLTRHTKIHLRQKDAAR

SEQ ID NO: 119 - protein, 178 aa, synthetic construct
MAERPFQCRICMRNFSDRSSLTTHIRTHTGEKPFACDICGRKFADRSHLARHTKIHTGSQKPFQCRICMRNFSRSDNLARHIRTHTGEKPFACDICGRKFAQSAHLARHTKIHTGSQKPFQCRICMRNFSQSGNLARHIRTHTGEKPFACDICGRKFARSDHLSRHTKIHLRQKDAAR

SEQ ID NO: 120 - protein, 178 aa, synthetic construct
MAERPFQCRICMRNFSRSDNLSEHIRTHTGEKPFACDICGRKFAQSANRNKHTKIHTGSQKPFQCRICMRNFSRSDNLARHIRTHTGEKPFACDICGRKFAWRGDRVKHTKIHTGSQKPFQCRICMRNFSDRSDLSRHIRTHTGEKPFACDICGRKFARRTDLRRHTKIHLRQKDAAR

SEQ ID NO: 121 - protein, 178 aa, synthetic construct
MAERPFQCRICMRNFSQSGSLSRHIRTHTGEKPFACDICGRKFARSDDLSRHTKIHTGSQKPFQCRICMRNFSRSDSLSQHIRTHTGEKPFACDICGRKFARSGHLSRHTKIHTGSQKPFQCRICMRNFSDRSDLSRHIRTHTGEKPFACDICGRKFAQSSTLARHTKIHLRQKDAAR

SEQ ID NO: 122 - protein, 178 aa, synthetic construct
MAERPFQCRICMRNFSDQSTLRSHIRTHTGEKPFACDICGRKFADRSNLSRHTKIHTGSQKPFQCRICMRNFSRSDALSEHIRTHTGEKPFACDICGRKFARSADRKRHTKIHTGSQKPFQCRICMRNFSQSGHLARHIRTHTGEKPFACDICGRKFATSSTLSKHTKIHLRQKDAAR

SEQ ID NO: 123 - protein, 178 aa, synthetic construct
MAERPFQCRICMRNFSRSDHLSRHIRTHTGEKPFACDICGRKFATSSHLARHTKIHTGSQKPFQCRICMRNFSTSGNLARHIRTHTGEKPFACDICGRKFATSGNLVRHTKIHTGSQKPFQCRICMRNFSRSDHLTRHIRTHTGEKPFACDICGRKFATSGHLVRHTKIHLRQKDAAR

SEQ ID NO: 124 - protein, 178 aa, synthetic construct
MAERPFQCRICMRNFSRSDNLARHIRTHTGEKPFACDICGRKFARSDHRKKHTKIHTGSQKPFQCRICMRNFSQSGNLARHIRTHTGEKPFACDICGRKFARSGNLRAHTKIHTGSQKPFQCRICMRNFSDSSALSTHIRTHTGEKPFACDICGRKFARSDHLSQHTKIHLRQKDAAR

SEQ ID NO: 125 - protein, 178 aa, synthetic construct
MAERPFQCRICMRNFSERSDLSVHIRTHTGEKPFACDICGRKFADRDDLSRHTKIHTGSQKPFQCRICMRNFSRSDDRKVHIRTHTGEKPFACDICGRKFADRSHRIRHTKIHTGSQKPFQCRICMRNFSRSDNLARHIRTHTGEKPFACDICGRKFAQSGHLARHTKIHLRQKDAAR

SEQ ID NO: 126 - protein, 178 aa, synthetic construct
MAERPFQCRICMRNFSQSSALTRHIRTHTGEKPFACDICGRKFARSDNLRAHTKIHTGSQKPFQCRICMRNFSQSGDLTRHIRTHTGEKPFACDICGRKFAQSGDLTRHTKIHTGSQKPFQCRICMRNFSQSGHLVTHIRTHTGEKPFACDICGRKFATSGNLVRHTKIHLRQKDAAR

SEQ ID NO: 127 - protein, 178 aa, synthetic construct
MAERPFQCRICMRNFSERSALATHIRTHTGEKPFACDICGRKFADRSALARHTKIHTGSQKPFQCRICMRNFSQSAHLSRHIRTHTGEKPFACDICGRKFARSDALARHTKIHTGSQKPFQCRICMRNFSQSGDLTRHIRTHTGEKPFACDICGRKFATSGNLSRHTKIHLRQKDAAR

SEQ ID NO: 128 - protein, 178 aa, synthetic construct
MAERPFQCRICMRNFSDRSALTRHIRTHTGEKPFACDICGRKFAQSGDLTRHTKIHTGSQKPFQCRICMRNFSRSDSLTRHIRTHTGEKPFACDICGRKFATSGNLTRHTKIHTGSQKPFQCRICMRNFSTSGHLTRHIRTHTGEKPFACDICGRKFARSDALREHTKIHLRQKDAAR

SEQ ID NO: 129 - protein, 178 aa, synthetic construct
MAERPFQCRICMRNFSQSGDLTRHIRTHTGEKPFACDICGRKFAQSGDLTRHTKIHTGSQKPFQCRICMRNFSQSGHLSTHIRTHTGEKPFACDICGRKFATSGNLSRHTKIHTGSQKPFQCRICMRNFSQSGSLRAHIRTHTGEKPFACDICGRKFARSDNLSKHTKIHLRQKDAAR

SEQ ID NO: 130 - protein, 178 aa, synthetic construct
MAERPFQCRICMRNFSDRSHLTTHIRTHTGEKPFACDICGRKFADRSALTRHTKIHTGSQKPFQCRICMRNFSQSGDLTRHIRTHTGEKPFACDICGRKFARSDALAAHTKIHTGSQKPFQCRICMRNFSTSGNLTRHIRTHTGEKPFACDICGRKFATSGHLTRHTKIHLRQKDAAR

SEQ ID NO: 131 - protein, 178 aa, synthetic construct
MAERPFQCRICMRNFSRSDSLSVHIRTHTGEKPFACDICGRKFAQSGDLTRHTKIHTGSQKPFQCRICMRNFSTSGNLTRHIRTHTGEKPFACDICGRKFAQSTHLTSHTKIHTGSQKPFQCRICMRNFSQSGHLARHIRTHTGEKPFACDICGRKFAQLTHLNSHTKIHLRQKDAAR

SEQ ID NO: 132 - protein, 178 aa, synthetic construct
MAERPFQCRICMRNFSDRSALSRHIRTHTGEKPFACDICGRKFARSQHLQTHTKIHTGSQKPFQCRICMRNFSDRSALSDHIRTHTGEKPFACDICGRKFAQSGNLARHTKIHTGSQKPFQCRICMRNFSRSDNLARHIRTHTGEKPFACDICGRKFAQSGHLARHTKIHLRQKDAAR

SEQ ID NO: 133 - protein, 178 aa, synthetic construct
MAERPFQCRICMRNFSQSSNLSRHIRTHTGEKPFACDICGRKFARSDHLSTHTKIHTGSQKPFQCRICMRNFSRSDTLSEHIRTHTGEKPFACDICGRKFAQSATLTRHTKIHTGSQKPFQCRICMRNFSRSDNLARHIRTHTGEKPFACDICGRKFAQSGALSRHTKIHLRQKDAAR

SEQ ID NO: 134 - protein, 178 aa, synthetic construct
MAERPFQCRICMRNFSERGTLARHIRTHTGEKPFACDICGRKFARSDHRKTHTKIHTGSQKPFQCRICMRNFSDRSNLTRHIRTHTGEKPFACDICGRKFARSDNLARHTKIHTGSQKPFQCRICMRNFSRSDNLARHIRTHTGEKPFACDICGRKFATSANLVRHTKIHLRQKDAAR

SEQ ID NO: 135 - protein, 178 aa, synthetic construct
MAERPFQCRICMRNFSDRSNLSRHIRTHTGEKPFACDICGRKFARSDNLTRHTKIHTGSQKPFQCRICMRNFSRSDTLSVHIRTHTGEKPFACDICGRKFADNSTRIKHTKIHTGSQKPFQCRICMRNFSRSDHLSNHIRTHTGEKPFACDICGRKFADNRDRIKHTKIHLRQKDAAR

SEQ ID NO: 136 - protein, 178 aa, synthetic construct
MAERPFQCRICMRNFSDRSNLSRHIRTHTGEKPFACDICGRKFAQLASRTKHTKIHTGSQKPFQCRICMRNFSRSDNLSAHIRTHTGEKPFACDICGRKFATSQNRITHTKIHTGSQKPFQCRICMRNFSRSDNLSRHIRTHTGEKPFACDICGRKFAQSGNLVRHTKIHLRQKDAAR

SEQ ID NO: 137 - protein, 178 aa, synthetic construct
MAERPFQCRICMRNFSQSGSLTRHIRTHTGEKPFACDICGRKFARSDALARHTKIHTGSQKPFQCRICMRNFSTSSNLTRHIRTHTGEKPFACDICGRKFATSGHLTRHTKIHTGSQKPFQCRICMRNFSRSDHLSEHIRTHTGEKPFACDICGRKFADSRDRTKHTKIHLRQKDAAR

SEQ ID NO: 138 - protein, 178 aa, synthetic construct
MAERPFQCRICMRNFSDRSALARHIRTHTGEKPFACDICGRKFAQSGNLARHTKIHTGSQKPFQCRICMRNFSRSDNLARHIRTHTGEKPFACDICGRKFAQSGHLARHTKIHTGSQKPFQCRICMRNFSRSDSLTQHIRTHTGEKPFACDICGRKFAQSSHLTRHTKIHLRQKDAAR

SEQ ID NO: 139 - protein, 178 aa, synthetic construct
MAERPFQCRICMRNFSDRSHLSEHIRTHTGEKPFACDICGRKFARSDARKTHTKIHTGSQKPFQCRICMRNFSRSDTLSTHIRTHTGEKPFACDICGRKFADRSNLTRHTKIHTGSQKPFQCRICMRNFSRSDNLARHIRTHTGEKPFACDICGRKFARKDNRITHTKIHLRQKDAAR

SEQ ID NO: 140 - protein, 178 aa, synthetic construct
MAERPFQCRICMRNFSQSGHLARHIRTHTGEKPFACDICGRKFAQSGNLTRHTKIHTGSQKPFQCRICMRNFSQSGDLTRHIRTHTGEKPFACDICGRKFAQSGHLARHTKIHTGSQKPFQCRICMRNFSQSGHLSQHIRTHTGEKPFACDICGRKFAQSTSLSKHTKIHLRQKDAAR

SEQ ID NO: 141 - protein, 178 aa, synthetic construct
MAERPFQCRICMRNFSTSSNLSAHIRTHTGEKPFACDICGRKFARSDNRKKHTKIHTGSQKPFQCRICMRNFSQNSNLRRHIRTHTGEKPFACDICGRKFAQSGNLTRHTKIHTGSQKPFQCRICMRNFSQLGSLSRHIRTHTGEKPFACDICGRKFALKSNLRRHTKIHLRQKDAAR

SEQ ID NO: 142 - protein, 178 aa, synthetic construct
MAERPFQCRICMRNFSQSGNLQAHIRTHTGEKPFACDICGRKFAQSGNLRAHTKIHTGSQKPFQCRICMRNFSQSGNLTRHIRTHTGEKPFACDICGRKFALKQHLRTHTKIHTGSQKPFQCRICMRNFSQSSALSRHIRTHTGEKPFACDICGRKFARSDSLRQHTKIHLRQKDAAR

SEQ ID NO: 143 - protein, 178 aa, synthetic construct
MAERPFQCRICMRNFSRSDHLSEHIRTHTGEKPFACDICGRKFARSDHRKRHTKIHTGSQKPFQCRICMRNFSTSSALSRHIRTHTGEKPFACDICGRKFARSDNLAAHTKIHTGSQKPFQCRICMRNFSTSGNLARHIRTHTGEKPFACDICGRKFAQSGDLTRHTKIHLRQKDAAR

SEQ ID NO: 144 - protein, 178 aa, synthetic construct
MAERPFQCRICMRNFSRSWKLQAHIRTHTGEKPFACDICGRKFAQSGNLTRHTKIHTGSQKPFQCRICMRNFSRSGHLSRHIRTHTGEKPFACDICGRKFARSDNLRAHTKIHTGSQKPFQCRICMRNFSTSSNLSRHIRTHTGEKPFACDICGRKFARSDNLTQHTKIHLRQKDAAR

SEQ ID NO: 145 - protein, 178 aa, synthetic construct
MAERPFQCRICMRNFSQSGDLTRHIRTHTGEKPFACDICGRKFALRTSLRKHTKIHTGSQKPFQCRICMRNFSTPSYLPTHIRTHTGEKPFACDICGRKFADRSALARHTKIHTGSQKPFQCRICMRNFSQSSHLTRHIRTHTGEKPFACDICGRKFARSDALARHTKIHLRQKDAAR

SEQ ID NO: 146 - protein, 178 aa, synthetic construct
MAERPFQCRICMRNFSQSGNLSRHIRTHTGEKPFACDICGRKFARSDALRNHTKIHTGSQKPFQCRICMRNFSRSGNLARHIRTHTGEKPFACDICGRKFARSDNRITHTKIHTGSQKPFQCRICMRNFSRSDNLSAHIRTHTGEKPFACDICGRKFARSDNRITHTKIHLRQKDAAR

SEQ ID NO: 147 - protein, 178 aa, synthetic construct
MAERPFQCRICMRNFSRSDNLARHIRTHTGEKPFACDICGRKFAQSANRTKHTKIHTGSQKPFQCRICMRNFSRSDNLARHIRTHTGEKPFACDICGRKFAQWNYRGSHTKIHTGSQKPFQCRICMRNFSQSSDLSRHIRTHTGEKPFACDICGRKFAHRSNLSKHTKIHLRQKDAAR

SEQ ID NO: 148 - protein, 178 aa, synthetic construct
MAERPFQCRICMRNFSQSGHLARHIRTHTGEKPFACDICGRKFATSGNLARHTKIHTGSQKPFQCRICMRNFSQSGNLTRHIRTHTGEKPFACDICGRKFARSDNRITHTKIHTGSQKPFQCRICMRNFSRSANLSRHIRTHTGEKPFACDICGRKFAQSDTRITHTKIHLRQKDAAR

SEQ ID NO: 149 - protein, 178 aa, synthetic construct
MAERPFQCRICMRNFSQRSNLSRHIRTHTGEKPFACDICGRKFATSGHLSRHTKIHTGSQKPFQCRICMRNFSRSDTLSEHIRTHTGEKPFACDICGRKFAANSNRIKHTKIHTGSQKPFQCRICMRNFSRSDNLSVHIRTHTGEKPFACDICGRKFARRDTRNNHTKIHLRQKDAAR

SEQ ID NO: 150 - protein, 178 aa, synthetic construct
MAERPFQCRICMRNFSRSDTLSEHIRTHTGEKPFACDICGRKFAQNANRIKHTKIHTGSQKPFQCRICMRNFSRSDSLSQHIRTHTGEKPFACDICGRKFARSHNRKTHTKIHTGSQKPFQCRICMRNFSRSDNLSRHIRTHTGEKPFACDICGRKFATNQNRITHTKIHLRQKDAAR

SEQ ID NO: 151 - protein, 178 aa, synthetic construct
MAERPFQCRICMRNFSERGTLSRHIRTHTGEKPFACDICGRKFARSDHLSRHTKIHTGSQKPFQCRICMRNFSQSSTRKTHIRTHTGEKPFACDICGRKFATSSTLSNHTKIHTGSQKPFQCRICMRNFSRSDHLSQHIRTHTGEKPFACDICGRKFAQSATRKTHTKIHLRQKDAAR

SEQ ID NO: 152 - protein, 178 aa, synthetic construct
MAERPFQCRICMRNFSTSDHLSEHIRTHTGEKPFACDICGRKFAQSASRTKHTKIHTGSQKPFQCRICMRNFSQSGSLTRHIRTHTGEKPFACDICGRKFAQSGNLRKHTKIHTGSQKPFQCRICMRNFSDRSDLSRHIRTHTGEKPFACDICGRKFAQSSDLSRHTKIHLRQKDAAR

SEQ ID NO: 153 - protein, 178 aa, synthetic construct
MAERPFQCRICMRNFSRSDNLARHIRTHTGEKPFACDICGRKFARSDALARHTKIHTGSQKPFQCRICMRNFSRSDALSEHIRTHTGEKPFACDICGRKFARSSDRTKHTKIHTGSQKPFQCRICMRNFSRSDHLSRHIRTHTGEKPFACDICGRKFARNQDRTNHTKIHLRQKDAAR

SEQ ID NO: 154 - protein, 178 aa, synthetic construct
MAERPFQCRICMRNFSRSDTLSVHIRTHTGEKPFACDICGRKFADRSHLSRHTKIHTGSQKPFQCRICMRNFSRSDNLSVHIRTHTGEKPFACDICGRKFADRGNLTRHTKIHTGSQKPFQCRICMRNFSRSDNLARHIRTHTGEKPFACDICGRKFADSHHRITHTKIHLRQKDAAR

SEQ ID NO: 155 - protein, 178 aa, synthetic construct
MAERPFQCRICMRNFSQSGNLSRHIRTHTGEKPFACDICGRKFARSDALATHTKIHTGSQKPFQCRICMRNFSRSDNLSTHIRTHTGEKPFACDICGRKFARSGNLSRHTKIHTGSQKPFQCRICMRNFSRSDNLSTHIRTHTGEKPFACDICGRKFATSSNRTKHTKIHLRQKDAAR

SEQ ID NO: 156 - protein, 178 aa, synthetic construct
MAERPFQCRICMRNFSDRSALSRHIRTHTGEKPFACDICGRKFARSDALARHTKIHTGSQKPFQCRICMRNFSPCRYRLDHIRTHTGEKPFACDICGRKFARSANLTRHTKIHTGSQKPFQCRICMRNFSRSDNLSTHIRTHTGEKPFACDICGRKFADNSYLPRHTKIHLRQKDAAR

SEQ ID NO: 157 - protein, 178 aa, synthetic construct
MAERPFQCRICMRNFSNSGDLTRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSQSGNLTRHIRTHTGEKPFACDICGRKFAQSGDLGEHTKIHLRQKDAAR

SEQ ID NO: 158 - protein, 178 aa, synthetic construct
MAERPFQCRICMRNFSRSDNLSRHIRTHTGEKPFACDICGRKFARSDARTRHTKIHTGSQKPFQCRICMRNFSDRSDLARHIRTHTGEKPFACDICGRKFARSSDLSRHTKIHTGSQKPFQCRICMRNFSRSDHLSRHIRTHTGEKPFACDICGRKFADSQDRKTHTKIHLRQKDAAR

SEQ ID NO: 159 - protein, 178 aa, synthetic construct
MAERPFQCRICMRNFSQSGSLTRHIRTHTGEKPFACDICGRKFAHRQHLQTHTKIHTGSQKPFQCRICMRNFSDRSNLSRHIRTHTGEKPFACDICGRKFALKQVLVRHTKIHTGSQKPFQCRICMRNFSRSDSLLRHIRTHTGEKPFACDICGRKFADRSNRRKHTKIHLRQKDAAR

SEQ ID NO: 160 - protein, 178 aa, synthetic construct
MAERPFQCRICMRNFSRSDDLTAHIRTHTGEKPFACDICGRKFAQSAHRKRHTKIHTGSQKPFQCRICMRNFSTSAHLARHIRTHTGEKPFACDICGRKFATSASRTRHTKIHTGSQKPFQCRICMRNFSTSGHLTRHIRTHTGEKPFACDICGRKFARSDALAQHTKIHLRQKDAAR

SEQ ID NO: 161 - protein, 178 aa, synthetic construct
MAERPFQCRICMRNFSRSGNLTAHIRTHTGEKPFACDICGRKFAQNANRIKHTKIHTGSQKPFQCRICMRNFSQSGSLTQHIRTHTGEKPFACDICGRKFATSQNLTRHTKIHTGSQKPFQCRICMRNFSTSDSLLRHIRTHTGEKPFACDICGRKFATSHHLTRHTKIHLRQKDAAR

SEQ ID NO: 162 - protein, 178 aa, synthetic construct
MAERPFQCRICMRNFSQSSTRKTHIRTHTGEKPFACDICGRKFATSSTRTKHTKIHTGSQKPFQCRICMRNFSDRSALSRHIRTHTGEKPFACDICGRKFAQSGDLTRHTKIHTGSQKPFQCRICMRNFSRSDSLARHIRTHTGEKPFACDICGRKFATSGNLARHTKIHLRQKDAAR

SEQ ID NO: 163 - protein, 178 aa, synthetic construct
MAERPFQCRICMRNFSTSGHLKEHIRTHTGEKPFACDICGRKFAQSGHLSRHTKIHTGSQKPFQCRICMRNFSQSSDLSRHIRTHTGEKPFACDICGRKFAQSGNLSKHTKIHTGSQKPFQCRICMRNFSQSGHLNRHIRTHTGEKPFACDICGRKFAQSGNLARHTKIHLRQKDAAR

SEQ ID NO: 164 - protein, 178 aa, synthetic construct
MAERPFQCRICMRNFSTSSNRKTHIRTHTGEKPFACDICGRKFAQSANRITHTKIHTGSQKPFQCRICMRNFSQSGSLTRHIRTHTGEKPFACDICGRKFALKQNRIKHTKIHTGSQKPFQCRICMRNFSTPSYLPTHIRTHTGEKPFACDICGRKFADRSALARHTKIHLRQKDAAR

SEQ ID NO: 165 - protein, 178 aa, synthetic construct
MAERPFQCRICMRNFSRSGTLTRHIRTHTGEKPFACDICGRKFAQSGDLTRHTKIHTGSQKPFQCRICMRNFSQSGHLARHIRTHTGEKPFACDICGRKFARRSTRKSHTKIHTGSQKPFQCRICMRNFSQSGNLARHIRTHTGEKPFACDICGRKFAQSGNLARHTKIHLRQKDAAR

SEQ ID NO: 166 - protein, 178 aa, synthetic construct
MAERPFQCRICMRNFSRSDHLSQHIRTHTGEKPFACDICGRKFARSAVRKNHTKIHTGSQKPFQCRICMRNFSRSDHLSEHIRTHTGEKPFACDICGRKFAQSHHRKTHTKIHTGSQKPFQCRICMRNFSDRSNLSRHIRTHTGEKPFACDICGRKFALKQHLNEHTKIHLRQKDAAR

SEQ ID NO: 167 - protein, 178 aa, synthetic construct
MAERPFQCRICMRNFSDRSNLSRHIRTHTGEKPFACDICGRKFARSDDRKTHTKIHTGSQKPFQCRICMRNFSERGTLARHIRTHTGEKPFACDICGRKFAQSGHLSRHTKIHTGSQKPFQCRICMRNFSQSGHLARHIRTHTGEKPFACDICGRKFAVSHHLRDHTKIHLRQKDAAR

SEQ ID NO: 168 - protein, 178 aa, synthetic construct
MAERPFQCRICMRNFSQSGNLARHIRTHTGEKPFACDICGRKFARLDILQQHTKIHTGSQKPFQCRICMRNFSRSDVLSEHIRTHTGEKPFACDICGRKFATRNGLKYHTKIHTGSQKPFQCRICMRNFSQSSDLSRHIRTHTGEKPFACDICGRKFARKYYLAKHTKIHLRQKDAAR

SEQ ID NO: 169 - protein, 235 aa, synthetic construct
LEPGEKPYKCPECGKSFSTSGNLVRHQRTHTGEKPYKCPECGKSFSTSGNLVRHQRTMAERPFQCRICMRNFSRSAHLSRHIRTHTGEKPFACDICGRKFATSGHLSRHTKIHTGSQKPFQCRICMRNFSRSDALSEHIRTHTGEKPFACDICGRKFAQNATRTKHTKIHTGSQKPFQCRICMRNFSTSGHLSRHIRTHTGEKPFACDICGRKFAQSGDLTRHTKIHLRQKDAAR

SEQ ID NO: 170 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 171 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 172 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 173 - protein, 10 aa, synthetic construct
  VARIANT 1..10: note = This sequence may encompass 1-10 residues
GGGGGGGGGG

SEQ ID NO: 174 - protein, 50 aa, synthetic construct
  VARIANT 1..50: note = This sequence may encompass 1-10 EAAAK repeating units
EAAAKEAAAKEAAAKEAAAKEAAAKEAAAKEAAAKEAAAKEAAAKEAAAK

SEQ ID NO: 175 - protein, 106 aa, synthetic construct
  VARIANT 2..51: note = This region may encompass 1-10 EAAAK repeating units
  VARIANT 56..105: note = This region may encompass 1-10 EAAAK repeating units
AEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKA

SEQ ID NO: 176 - protein, 5 aa, synthetic construct
PAPAP

SEQ ID NO: 177 - protein, 12 aa, synthetic construct
AEAAAKEAAAKA

SEQ ID NO: 178 - protein, 68 aa, synthetic construct
  VARIANT 1..68: note = This sequence may encompass 10-34 AP repeating units
APAPAPAPAPAPAPAPAPAPAPAPAPAPAPAPAPAPAPAPAPAPAPAPAPAPAPAPAPAPAPAPAPAP

SEQ ID NO: 179 - protein, 6 aa, synthetic construct
GGGSGT

SEQ ID NO: 180 - protein, 6 aa, synthetic construct
GTGGGS

SEQ ID NO: 181 - protein, 8 aa, synthetic construct
GGGSGGGS